(19) United States
(12) Patent Application Publication
(10) Pub. No.: US 2006/0134691 A1
(43) Pub. Date: Jun. 22, 2006

(54) Labeling methodology comprising oligopeptides

(76) Inventors: Manfred Auer, Moedling (AT); Nicole-Claudia Meisner, Wien (AT); Jan-Marcus Seifert, Brunn am Gebirge (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/534,966

(22) PCT Filed: Dec. 4, 2003
(86) PCT No.: PCT/EP03/13715

§ 371 Date: May 16, 2005

(30) Foreign Application Priority Data
Dec. 5, 2002 (GB) 0228429.7

(51) Int. Cl. G01N 33/53; C07K 14/47

(52) U.S. Cl. 435/7.1; 530/409

(57) ABSTRACT

A method for providing a labeled target protein or labeled target peptide in high purity, which method comprises the steps of a. contacting a chemical molecule, which chemical molecule comprises a target protein or a target peptide residue beside amino acid residues, spacer residues, and/or linker residues, an affinity tagging residue, and a labeling residue which labeling residue may be detected by physical means, with an affinity support, to obtain said chemical molecule bound to the affinity support via the affinity tag of the affinity tagging residue, b. removing impurities in the reaction mixture surrounding the affinity support to which the chemical molecule according to step a. is bound, and c. cleaving or eluting said chemical molecule from said affinity support; and compounds useful in such method. Such method is e.g. useful for (ultra) HTS-screening, especially spectroscopic HTS systems at single molecule resolution.

The present invention relates to a labeling methodology comprising oligopeptides, e.g. for use in single molecule resolution confocal spectroscopy, multiplexing, imaging and (ultra) high throughput screening.

A broad range of biological and high throughput screening (=HTS) assay applications are based on selective detection of a target molecule, e.g. a protein which carries an appropriate label. This is usually achieved by incorporation of the label via an appropriate reactive group which allows selective reaction with certain groups on the target protein (e.g. reaction of a succinimidylester-activated dye with primary amines in the side chains of a protein). However, such conventional labeling approaches may face several major drawbacks: Mainly, the limited efficiency of most chemical reactions results in a certain percentage of remaining unreacted and therefore unlabeled molecules, which usually interferes with the measurement and should be quantitatively removed. As the biochemical properties of the target molecule should preferably not be dramatically changed by the introduction of such modification, e.g. introduction of a label, the separation of the two species, the labeled and the unlabeled molecules, by common biochemical methods is often not straightforward. Moreover, each molecule requires its own, optimized labeling strategy. In today's HTS environment, where a vast number of target molecules need to be prepared in parallel, ideally based on a common approach, this may form a significant hurdle. Another reason is the single molecule technique which became available during recent years for assay development and screening. In this technique preferably a 1:1 labeled protein Is used for high resolution mechanistic studies and multiplexed screening systems. Especially for solution assays and screening, proteins should be provided in a soluble form.

We have now established a labeling methodology (technology), e.g. for generating labeled, e.g. 1:1 labeled, preferably labeled and soluble, protein or peptide, e.g. recombinant protein, in high purity, applicable to virtually any desired label and target protein or target peptide sequence.

In one aspect the present invention provides a method for providing a labeled target protein or labeled target peptide, e.g. in high purity, which method comprises the steps of
a. contacting a chemical molecule, which chemical molecule comprises, optionally beside spacer and/or linker residues, the following parts
   a target protein or a target peptide residue which is covalently bound as one of both terminal groups of said chemical molecule to a chemical backbone, which chemical backbone consists of amino acids and/or spacers and optionally linkers, covalently bound to each other, and which backbone contains, optionally beside spacer and/or linker residues,
      one amino acid or spacer residue as the second of both terminal groups of said chemical molecule covalently bound to a reversible or irreversible affinity tagging residue, with the proviso that in case that the affinity tagging residue is an irreversible affinity tagging residue, there is at least one linker residue covalently bound to said amino acid or spacer residue, which amino acid or spacer residue is on the other hand covalently bound to the irreversible affinity tagging residue, and
      one amino acid or spacer residue covalently bound to a labeling residue which labeling residue may be detected by physical means,
   with an affinity support, to obtain said chemical molecule bound to the affinity support via the affinity tag of the affinity tagging residue,
b. removing impurities in the reaction mixture surrounding the affinity support to which the chemical molecule according to step a. is bound, and
c. cleaving or eluting said chemical molecule from said affinity support to obtain
c1 in case of an irreversible affinity tagging residue, a labeled target protein or labeled peptide comprising a chemical backbone, which backbone contains, optionally beside spacer and/or linker residues, one amino acid or spacer residue covalently bound to a labeling residue which labeling residue may be detected by physical means,
c2. in case of a reversible affinity tagging residue, a chemical backbone covalently bound to a protein or a peptide residue, which backbone contains, optionally beside spacer and/or linker residues,
   one amino acid or spacer residue covalently bound to a labeling residue which labeling residue may be detected by physical means, and
   one amino acid or spacer residue covalently bound to a reversible affinity tagging residue,
      e.g. in high purity, e.g. wherein the protein or peptide is labeled in a ratio of 1:1.

An affinity tagging group containing residue may be cleaved from the compound of the present invention obtained in step c2. as appropriate, if desired, and If a linker is present, according to, e.g. analogously, to a method as conventional.

A chemical molecule as used in a process provided by the present invention is herein also indicated as “CHEMICAL MOLECULE” or “a molecule of (according to) the present invention”.

Preferably a CHEMICAL MOLECULE comprises one affinity tagging residue, one labeling residue and at least one spacer residue.

Preferably the chemical backbone of a CHEMICAL MOLECULE comprises 5 to 30, e.g. 7 to 25, such as 10 to 15 units selected from amino acid, spacer and linker residues.

Preferably a CHEMICAL MOLECULE comprises the following number of residues in corresponding units, if present:
i. 1 to 10 amino acids and/or spacer residues, covalently bound to each other as described above, and carrying at one amino acid or spacer of such residue formed, as a terminal group of said CHEMICAL MOLECULE, an affinity tagging residue, which residue formed is covalently bound to a residue of ii.,
ii. 1 to 6 spacer residues, covalently bound to each other as described above, and covalently bound at one end of such residue formed to an amino acid or spacer residue of i., and covalently bound at the other end of such residue formed to a residue of iii.,
iii. 1 linker residue covalently bound at one end of said residue to a residue of ii., and covalently bound at the other end of said residue to a residue of iv.,
iv. 1 to 2 spacer residues covalently bound to each other, which residue formed is covalently bound at one end of said residue to a residue of iii. and covalently bound at the other end of said residue to an amino acid or spacer residue of v.,
v. 1 to 2 amino acid or spacer residues, covalently bound to each other, one amino acid or spacer of such residue formed carrying a labeling residue which labeling residue may be detected by physical means, and which residue formed is covalently bound at one end to a group of iv., and covalently bound at the other end to the target protein or target peptide residue.

More preferably at least one unit i. to v. is present in a CHEMICAL MOLECULE:

The term “amino acid residue” as used herein shall mean, that an amino acid residue is derived from an amino acid, which forms said amino acid residue upon covalent binding to one or both of its neighboring residues in a CHEMICAL MOLECULE.

The term “spacer residue” as used herein shall mean, that a spacer residue is derived from a spacer, which forms said spacer residue upon covalent binding to one or both of its neighboring residues in a CHEMICAL MOLECULE.

The term “linker residue” as used herein shall mean, that a linker residue is derived from a linker, which forms said linker residue upon covalent binding to one or both of its neighboring residues in a CHEMICAL MOLECULE.

The term “affinity tagging residue” as used herein shall mean, that an affinity tagging residue is derived from an affinity tag, which forms said affinity tagging residue upon covalent binding to an amino acid residue or a spacer residue in a CHEMICAL MOLECULE.

The term “labeling residue” as used herein shall mean, that a labeling residue is derived from a label, which forms said labeling residue upon covalent binding to an amino acid residue or a spacer residue in a CHEMICAL MOLECULE.

The term “target protein or target peptide residue” as used herein shall mean, that a target protein or target peptide residue is derived from a target protein or a target peptide, which forms said target protein or target peptide residue upon covalent binding to an amino acid residue or a spacer residue in a CHEMICAL MOLECULE.

Methods to react
an amino acid (residue) with another amino acid (residue), or with a spacer (residue), linker (residue), affinity tag (affinity tagging residue), label (labeling residue), target protein or target peptide (residue), or
a spacer (residue) with another spacer (residue), or with an amino acid (residue), linker (residue), affinity tag (affinity tagging residue), label (labeling residue), target protein or target peptide (residue),
   to obtain a CHEMICAL MOLECULE are known, or may be carried out as appropriate, e.g. according, e.g. analogously to a method as conventional, e.g. or as described herein.

In a method provided by the present invention an amino acid includes naturally occurring amino acids and other amino acids, e.g. modified amino acids, amino acid analogs or combinations thereof. Such amino acids may be in any isomeric form, e.g. In L- and/or D-form. A naturally occurring amino acid includes e.g. cysteine, glycine, lysine and threonine; other amino acids include e.g. aminoadipic acid, β-alanine, β-aminopropionic acid, amino-butyric acid, aminocaproic acid, hydroxylysine, norvaline, norleucine, ornithine. Amino acid, spacer and linker residue are covalently bound to each other via functional groups present in the corresponding amino acid, spacer or linker, respectively. Several amino acids bound covalently to each other via amide bonds originating from the reaction of a carboxylic group of one amino acid with an amine group of a second amino acids may be homopeptides, such as glycine-glycine; or heteropeptides, such as glycine-lysine-glycine.

In a method provided by the present invention the target protein or target peptide is the molecule which is to be labeled. A target protein or target peptide includes any (bio)chemical substance comprising amino acid residues, such as e.g. a receptor molecule, an enzyme, an antigen, an antibody; or any other substance of interest. The target protein or target peptide may be obtained as appropriate, e.g. according, e.g. analogously, to a method as conventional, e.g. by isolation from a biological source or by chemical or recombinant production.

In a method provided by the present invention, a spacer residue in a CHEMICAL MOLECULE serves to provide a distance between two parts of the CHEMICAL MOLECULE. A spacer residue is derived from an appropriate spacer, such as a spacer useful in combinatorial, peptide and oligonucleotide chemistry. A spacer includes spacers such as conventional, e.g. including a chemical molecule having, optionally beside a backbone which backbone is free of any functional group, at least one, preferably two functional groups, which functional groups are able to react with other functional groups of isolated parts of the CHEMICAL MOLECULE, e.g. in a production process for the preparation of such CHEMICAL MOLECULE. A spacer e.g. includes amino acids and other molecules having at least one, preferably two functional groups, such as (C₁₋₂₀)alkanes having one, preferably two, functional groups. Preferably a spacer is an amino acid, such as glycine, β-alanine, 4-amino-butyric acid, 8-amino-3,6-dioxaoctanoic acid, or an amino- or diamino-(C₁₋₂₀)alkane. Spacer residues derived from such spacers may be present in a CHEMICAL MOLECULE one- or more fold, e.g. in single or multiple copies. Optionally such spacer may comprise solubility enhancing residues. such as hydrophilic residues.

Such spacers are known or may be prepared as appropriate, e.g. according, e.g. analogously, to a method as conventional.

In another aspect the present invention provides a method for enhancing the solubility of a labeled target protein or target peptide, e.g. by binding to or introducing a hydrophilic spacer, e.g. an ADO group, or a hydrophilic affinity tagging group, such as e.g. Hexa-His or FLAG.

In a method provided by the present invention, a linker residue n a CHEMICAL MOLECULE serves to provide a cleavable position in a CHEMICAL MOLECULE. Such linker residue is derived from an appropriate linker, e.g. such as conventional, e.g. including photo cleavable linkers, such as a linker cleavable by light, also called light labile linker, e.g. a nitro aryl containing linker; chemically cleavable linkers, e.g. base labile and acid labile linkers, such as acid labile linkers; enzyme-cleavable linkers; redox-labile linkers; and masked linkers, e.g. benzyl, benzhydryl, benzhydrylidene, trityl, xanthenyl, benzoin, silicon or allyl based linkers; preferably photo cleavable linkers or chemically cleavable linkers. A photo cleavable linker may carry e.g. a nitro aryl group, such as e.g. 4-alkyl-5-nitro-benzoyl or α-methyl-6-nitroveratryl groups. An acid cleavable linker may comprise a Rink Amide (RAM) or an acid labile 2-chlorotrityl linker. Such linkers are known or may be prepared as appropriate, e.g. according, e.g. analogously, to a method as conventional.

In a method provided by the present invention the labeling residue in a CHEMICAL MOLECULE may be as appropriate, e.g. as conventional and may be detected by physical means. A labeling residue is derived from a labeled molecule, which label may be detected by physical means. A label includes e.g. a chelated ion or a dye which may be detected, e.g. by an enzymatic reaction, magnetic resonance detection or by fluorescent means, preferably by fluorescent means. Appropriate labeling molecules, e.g. chelating ions or dyes, are known or may be provided as conventional, e.g. such label is selected from the group consisting of fluorescein, tetramethylrhodamine, merocyanines e.g. Cy5®, ALEXA®, e.g. ALEXA 488®, acridines e.g. ATTO 4950 and Bodipy® or solvatochromic dyes such as described in Toutchkine A., Kraynov V. and Hahn K., J. Am. Chem. Soc. (2003), 125:4132-4145.

In a method provided by the present invention an affinity tagging group is derived from a chemical molecule comprising or being an affinity tag, which affinity tag is specifically recognized by another molecule, e.g. via a functional group, present in an affinity support. The affinity tag is bound to a spacer or amino acid in the CHEMICAL MOLECULE via functional groups of said chemical molecule comprising an affinity tag, e.g. via an NH₂ or COOH group, e.g. the affinity tagging group is bound via the ε-NH₂ group of a lysine residue to the CHEMICAL MOLECULE. A chemical molecule comprising or being an affinity tag includes such as appropriate, e.g. such as conventional, including an amino acid, and a peptide of 2 to 20 amino acids, preferably 2 to 10, which amino acids are covalently bound to each other via amide bonds originating from a carboxylic group of one amino acid with an amine group of a second amino acid and includes polyhistidine (His₅-His₆), polyarginine (Arg₅-Arg₆), FLAG (=DYKDDDDKGK), Strep-tag II (=WSHPQFEK), c-myc epitope (=EQKLISEEDL), S-tag (=KETAMKFERQHMDS, see e.g. Terpe K., Appl. Microbiol. Biotechnol. (2003) 60:523-533) or APP (=EFRH), biotin or an individual combination of cited molecules comprising or being an affinity tag. A chemical molecule comprising or being an affinity tag is preferably selected from the group consisting of amino acid, a peptide of 2 to 10 amino acids, biotin and an individual combination of one or more affinity tags cited.

Functional groups in amino acids, spacers, linkers, affinity tags, labels and target proteins or target peptides, useful for binding two parts of the CHEMICAL MOLECULE, e.g. include halogen, hydroxy, alkoxy, amino, carboxyl, acylamino groups.

In a method provided by the present invention an affinity support includes a solid affinity support, preferably coated. Preferably an affinity support comprises a substance selected from the group consisting of amino acid chelating agents, e.g. nickel-ion; anti-amino acid sequence antibodies; avidin and streptavidin.

Such affinity supports and appropriate affinity tags for use together with an individual, appropriate affinity support (designated herein also as “pairs of substances”), are known or may be provided as conventional, e.g. according, e.g. analogously to a method as conventional.

Preferably pairs of substances (affinity tag-affinity support) include Polyhistidine—Ni²⁺-NTA, Polyarginine-cation exchange resin, FLAG-anti-FLAG antibody, Strep-tag II-Strep-Tactin (=streptavidin variant with high affinity for Strep-tag II), c-myc epitope-anti c-myc antibody, S-tag-S-fragment of RNAse A, APP-anti APP antibody, biotin-avidin or biotin-streptavidin.

Such affinity tagging residue in a CHEMICAL MOLECULE may bind either reversibly or practically irreversible to an affinity support. “Reversibly” means that the affinity tag may also be removed from the affinity support when cleaving the CHEMICAL MOLECULE from said affinity support, thus remaining in the CHEMICAL MOLECULE after cleavage. “Irreversibly” means that the affinity tag remains on the affinity support when cleavage of the CHEMICAL MOLECULE from the affinity support is carried out and, that a chemical molecule obtained after cleavage is lacking such affinity tagging group.

Preferably the amino acid or spacer group in a CHEMICAL MOLECULE carrying the affinity tagging group is bound, optionally via a further spacer or amino acid (residue), to a linker (residue), in order to be able to remove the amino acid or spacer residue carrying the affinity tagging residue from the CHEMICAL MOLECULE via cleavage; e.g. in case of an reversible tagging group after cleavage of the CHEMICAL MOLECULE from the affinity support, or, in case of an irreversible tagging residue, directly at the affinity support, leaving the amino acid or spacer residue carrying the affinity tagging residue, optionally together with the linker residue, at the affinity support. In case of an irreversible tagging residue such linker residue must be present in a CHEMICAL MOLECULE, in case of a reversible affinity tagging residue such linker is present or is not present. E.g. in the case that the presence of an affinity tagging group is desired, e.g. in order to enhance the solubility of the target protein or target peptide in a CHEMICAL MOLECULE cleaved from the affinity support, it may be favorable that the affinity tagging group remains in a CHEMICAL MOLECULE obtained in step c1. and thus cleavage of the affinity tag residue and in consequence a linker residue is not necessarily present in a CHEMICAL MOLECULE.

Contact of the affinity support with the CHEMICAL MOLECULE for binding of the affinity tagging residue to the affinity support in step a. may be carried out as appropriate, e.g. according, e.g. analogously, to a method as conventional. Preferably an appropriate contacting solvent medium wherein the CHEMICAL MOLECULE is dissolved, is used. Such solvent medium includes appropriate buffer solutions, optionally in the presence of organic solvent, e.g. in order to enhance solubility of the CHEMICAL MOLECULE.

On contact of the CHEMICAL MOLECULE with the affinity support the CHEMICAL MOLECULE binds via its affinity tagging group to the affinity support.

Impurities in the contact solvent medium surrounding the affinity support to which the CHEMICAL MOLECULE obtained in step b is bound, conveniently may be carried out by removing the contact solvent medium and washing the affinity support with appropriate solvent medium.

Elution of the CHEMICAL MOLECULE from the affinity support may be carried out as appropriate, e.g. depending on the chemical nature of the pair of substances (affinity tag-affinity support) used, e.g. by using a solvent medium having a different pH compared to the contact solvent medium (pH change), e.g. or by use of a base, such as imidazole. In case of an irreversible affinity tag, the amino acid or spacer residue carrying the affinity tagging residue is cleaved off via the linker residue which is present in that case in the CHEMICAL MOLECULE: The cleavage conditions of such linker residue are depending on the chemical nature of the linker. Cleavage of a linker may be carried out as appropriate, e.g. as described above, such as photo chemically, or chemically.

In case that a reversible affinity tagging residue and a linker residue is present in a CHEMICAL MOLECULE eluted from the affinity support, said affinity tagging group may be cleaved from the CHEMICAL MOLECULE via the cleavable linker, e.g. as described above, e.g. in appropriate cleaving medium, e.g. comprising a buffer medium, optionally in the presence of organic solvent. Purification of the CHEMICAL MOLECULE cleaved from the affinity tagging group, such as separation of the CHEMICAL MOLECULE from the linker residue, amino acid or spacer residue and affinity tag residue obtained after cleavage, may be carried out as appropriate, e.g. by use of size exclusion chromatography, such as by gel filtration.

The thus obtained CHEMICAL MOLECULE, optionally missing the affinity tag, may be obtained in high purity, e.g. 90% to 100% such as 95% to 100%, e.g. 97% to 100%, such as 99% to 100%, e.g. 100% purity.

In another aspect the present invention provides a method for providing a labeled target protein or labeled target peptide, e.g. in high purity, which method comprises the steps of
1. reacting a compound, which compound comprises, optionally beside spacer and/or linker residues, the following parts
   one amino acid or spacer residue as one of both terminal groups of said chemical molecule covalently bound to a reversible or irreversible affinity tagging residue, with the proviso that in case that the affinity tagging residue is an irreversible affinity tagging residue, there is at least one linker residue covalently bound to said amino acid or spacer residue, which amino acid or spacer residue is on the other hand covalently bound to the irreversible affinity tagging residue,
   one amino acid or spacer residue covalently bound to a labeling residue which labeling residue may be detected by physical means,
   one amino acid or spacer residue covalently bound to a reactive group containing residue, which residue is covalently bound as one of both terminal groups of said compound, and wherein the reactive group is able to react with a functional group contained in a target protein or target peptide,
   with a target peptide or target protein,
2. contacting a chemical molecule obtained in step 1. with an affinity support, to obtain said chemical molecule bound to the affinity support via the affinity tag of the affinity tagging residue,
3. removing impurities in the reaction mixture surrounding the affinity support to which the chemical molecule according to step a. is bound, and
4. cleaving or eluting said chemical molecule from said affinity support to obtain
4a. in case of an irreversible affinity tagging residue, a labeled target protein or labeled target peptide comprising a chemical backbone, which backbone contains, optionally beside spacer and/or linker residues, one amino acid or spacer residue covalently bound to a labeling residue which labeling residue may be detected by physical means,
4.b in case of a reversible affinity tagging residue, a chemical backbone covalently bound to a protein or a peptide residue, which backbone contains, optionally beside spacer and/or linker residues,
   one amino acid or spacer residue covalently bound to a labeling residue which labeling residue may be detected by physical means,
   one amino acid or spacer residue covalently bound to a labeling residue, which labeling residue may be detected by physical means, and which labeling residue or spacer residue is covalently bound to a reversible affinity tagging residue.

An affinity tagging group containing residue may be cleaved from the compound of the present invention obtained in step 4b. as appropriate, if desired, and if a linker is present, according to, e.g. analogously, to a method as conventional.

A chemical compound as produced in process step 1. or as used in step a. in a method of the present invention is herein also indicated as “CHEMICAL COMPOUND”, or as compound of (according to) the present invention.

A reactive group of a molecule of the present invention includes a functional reactive group, which is able to react covalently with another functional group of the target protein or the target peptide. A target protein or a target peptide contains such functional (reactive) group, e.g. via introduction as appropriate, e.g. according, e.g. analogously, to a method as conventional.

Preferably such reactive group includes, e.g. is selected from the group consisting of,
halogen, SH, OH, NH₂, ethinyl, azide, o-(diphenylphosphine) phenylester,
a group containing a reactive double bond, e.g. a maleimido group, or a vinyl-sulfonyl group,
a group which forms together with a carbonyl group of the residue to which the reactive group is bound, a carboxylic acid derivative (e.g. said carbonyl group may be derived from an amino acid residue or a spacer or linker (residue) comprising a carboxylic acid), the reactive group together with the carbonyl group of said amino acid function forms a group CO-Z, wherein Z is the reactive group and is S—R, O—R, NH—R, or halogen, and
R is
   (C₁₋₈)alkyl, such as (C₁₋₄)alkyl, e.g. including unsubstituted and substituted (C₁₋₈)alkyl, e.g. (C₁₋₈)alkyl substituted by
      (C₁₋₈)aryl, e.g. phenyl(C₁₋₄)alkyl, such as benzyl; halogen, such as bromomethyl or iodomethyl;
      hydroxy and/or mercapto, such as —CH₂—CH(OH)—CH(OH)—CH₂—SH,
      —SO₃H or —SO₃Na, e.g. (NaO)SO₂—(C₂₋₄)alkyl, such as 2-sodiumsulfonylethyl,
         (C₆₋₁₈)aryl, such as phenyl,

wherein a (C₆₋₁₈)aryl is unsubstituted or substituted, e.g. benzyl substituted by NH—CO—CH₃, or

the carbonyl group together with Z forms a carboxylic acid anhydride.

Preferably the reactive group containing residue is
a cysteine residue and the reactive group is the —SH group contained therein,
a maleimido-(C₁₋₆)alkyl carboxylic acid residue and the reactive group is the double bond of the maleimido group, or
an amino acid residue, e.g. a glycine residue, and the reactive group is an S—R group, wherein R is as defined above and which R forms together with a carbonyl group of said residue a thioester.

Halogen includes fluoro, chloro, iodo and bromo, e.g. iodo or bromo.

Reaction of a compound of the present invention with a target protein or target peptide may be carried out via reaction of the reactive group in a compound of the present invention, with a functional group present in such target protein or target peptide, e.g. as appropriate, e.g. according, e.g. analogously, to a method as conventional, or as described herein.

E.g. if the reactive group containing residue
is an —SH group, e.g. the —SH group of a cysteine, and the target protein or target peptide contains a C-terminal thioester group, transesterification may occur, and a novel thioester bond between said residue and the target protein or target peptide may be formed in a transesterification reaction; and a similar reaction may happen vice versa, e.g. in case that the reactive group together with a carbonyl group of the residue to which the reactive group is bound, forms a thioester group and the target protein or target peptide contains an —SH group;
is a maleimido group, or a vinyl-sulfonyl group, or halogen,
e.g. bound to a spacer residue, and a target protein or a target peptide has at least one internal —SH function, e.g. an internal cysteine residue, or a N-terminal —SH function, preferably one single —SH function, a thioether bond between said residue and the target protein or target peptide may be formed by said reaction;
is an aldehyde and the target protein or target peptide contains a terminal amine group e.g. in a cysteine, serine or threonine, an imine bond may be formed between a target protein or target peptide, with subsequent rearrangement to obtain a heterocyclic ring (see e.g. J. P. Tam, Q. Yu, Z. Miao, Biopolymers (1999), 51, 311-332);
forms together with a carbonyl group of the residue of a spacer or of an amino acid to which it is bound, a carboxylic acid derivative, e.g. a thioester, said ester forming group may react with the —SH-group of an N-terminal cysteine containing target protein or target peptide, e.g. via transesterification and subsequent rearrangement to obtain an amide bond between a target protein or target peptide and the residue of a CHEMICAL COMPOUND;
is an ethinyl group and the target protein or target peptide contains an azide group, e.g. introduced as a non natural methionine analogue (see e.g. Kiick K. et al., PNAS early edition) a 1,2,3-triazole 5-membered ring may be formed between said residue and the target protein or target peptide by cycloaddition (Click-Chemistry, e.g. Kolb H. C. et al, Angewandte Chemie, Int. Ed. (2001) 40(11):2004-2021);
is an azide group and the target protein or target peptide contains an ethinyl group, e.g. introduced as a non natural methionine analogue (see e.g. Kiick K. et al., PNAS early edition) a 1,2,3-triazole 5-membered ring may be formed between said residue and the target protein or target peptide by cycloaddion (Click-Chemistry, e.g. Kolb H. C. et al, Angewandte Chemie, Int. Ed (2001) 40(11):2004-2021);
is an o-(diphenylphosphin) phenylester and the target protein or target peptide contains an azide group, e.g. introduced as a non natural methionine analogue (see e.g. Kiick K. et al., PNAS early edition), an amide bond may be formed between said residue and the target protein or target peptide by aza-ylide formation and intramolecular acyl substitution (Staudinger-ligation, see e.g. Merkx R. et al., Tet. Lett (2003), 44:4515-4518 and Kiick K. et al., PNAS early edition).

If a target protein or target peptide contains one single —SH group, e.g. in a cysteine group of said target protein or target peptide, and the reactive group in the reactive group containing residue is a double bond, e.g. a double bond of an maleimido or vinylsulfonyl group, the —SH group of the cysteine residue may be added to said double bond and a thioether is formed. In case said —SH group is the —SH-group of a C-terminal cysteine an amide bond may be formed on reaction of the target protein or target peptide with a thioester as the reactive group bound to said residue via transesterification to a thioester and subsequent rearrangement.

For binding a target protein or target peptide to a compound of the present invention, one or more natural or non natural amino acid, e.g. a methionine analogue, a threonine or a cysteine, may be introduced into said target protein or target peptide, if desired. this may be carried out as appropriate, such as according, e.g. analogously, to a method as conventional, e.g. by chemical or recombinant methods.

Generally, an aldehyde group as a reactive group at an amino acid or spacer residue, may be obtained by reaction of a bifunctional reagent, having two identical functional groups, e.g. a dicarboxylic acid or a dicarboxylic ester, preferably in an activated form, such as squaric acid diethyl ester, with an amine group of a spacer residue or an amino acid residue, to obtain an amide bond between said spacer or an amino acid residue and one function of said bifunctional reagents, and coupling the second functional group thereof to the amino group of an amino acid ester aldehyde acetal or an amino(C₁₋₁₀)alkyl carboxylic acid ester aldehyde acetal. Acid treatment of an intermediate obtained removes the acetal to form the terminal aldehyde function and an aldehyde group is obtained. Alternatively the amino group of an amino acid or an amino(C₁₋₁₀)alkyl carboxylic acid is coupled to the second function of the bifunctional reagent and converted into an glycol aldehyde acetal ester by reaction of the carboxylic acid salt with bromo acetaldehyde dimethyl acetal, followed by acetal removal with an acid to obtain the terminal aldehyde (see e.g. Liu C. F., Tam J. P., Proc. Natl. Acad. Sci. (1994) 91, 6584-6588; Liu C. F., Tam J. P., J. Amer. Chem. Soc. (1994)10, 4149-4153).

We have further found specific compounds of the present invention, which are useful in a method provided by the present invention.

In another aspect the present invention provides a compound of the present invention, which compound comprises the following parts
A. a reactive group containing residue, which residue is covalently bound as one of both terminal groups of said compound, and wherein the reactive group is selected from the group consisting of thiol, halogen, imine, aldehyde, the double bond in a vinylsulfonyl or a maleimido group and a group which forms together with a carbonyl group of said residue a carboxylic acid derivative,
B. a label containing residue, which label may be detected by physical means,
C. an affinity tagging group containing residue, which residue is covalently bound as the second of both terminal residues in a CHEMICAL COMPOUND, and wherein said affinity tagging group is a reversible affinity tagging group or an irreversible affinity tagging group, with the proviso that in case that the affinity tagging group is an irreversible affinity tagging group, there is at least one linker residue covalently bound to an amino acid or spacer residue, which carries the irreversible affinity tagging group, and
which residue of any of A., B. or C. is selected from
   1 to 10 amino acids, which amino acids are covalently bound to each other via amide bonds of the carboxylic acid group of one amino acid and the amine group of a second amino acid,
   1 to 10 spacers, which spacers are covalently bound to each other via functional groups of said spacers, and
   1 to 10 amino acids and spacers, which amino acid and spacers are covalently bound to each other either via amide bonds of the carboxylic acid group of one amino acid and the amine group of a second amino acid, or via functional groups of said spacers, or via an amine group or a carboxylic acid group of an amino acid with a functional group of a spacer,
D. optionally further spacer residues,
E. optionally linker residues,
   e.g. wherein
   amino acid residue, spacer residue, linker residue, affinity tagging group (=affinity tagging residue) label (resulting in a labeling residue when bound to a CHEMICAL MOLECULE) and reactive group are as defined above.

A CHEMICAL COMPOUND provided by the present invention is herein also indicated as “a compound of (according to) the present invention”.

In a compound of the present invention and in a molecule of the present invention, with the exception of the reactive group (A in a compound of the present invention), the parts of said compound or molecule, i.e. spacers, linkers, amino acids, labeling group and affinity tagging group are preferably bound to each other or to said compound or molecule via amide bonds. Preferably a compound of the present invention comprises in total 5 to 30, e.g. 7 to 25, such as 10 to 15 residues selected from amino acid, spacer and linker residues defined in any of A., B., C, D and E.

In another aspect the present invention provides a compound of the present invention which is covalently bound to the residue of a target protein or a target peptide via a bond originating from the reaction of a reactive group defined in A., with a functional group of said target protein or target peptide, e.g. which is a molecule of the present invention, e.g. wherein a target protein or a target peptide is as defined above, e.g. and wherein the bond between said target protein or target peptide to a compound of the present invention is as defined above.

In another aspect the present invention provides a compound of the present invention which is covalently bound to the residue of a target protein or a target peptide via a bond originating from the reaction of a reactive group defined in A., with a functional group of said target protein or target peptide, e.g. which is a molecule of the present invention, which molecule is further bound to an affinity support via the affinity tagging group as defined in C., e.g. and wherein the affinity support is as defined above, e.g. and wherein the bond of a molecule of the present invention to the affinity support is as defined above.

Preferably in a compound of the present invention at least one spacer residue is present, e.g. one spacer residue covalently bound between the reactive group containing residue and/or the label containing residue and/or affinity tag containing residue. Preferably in a compound of the present invention one linker residue is present, e.g. covalently bound to that residue which is on the other hand bound to the affinity tagging group. If not otherwise defined herein, a functional group is preferably a reactive group.

In another aspect the present invention provides a compound of the present invention which is a compound of formula
A-Y-D-Y′—X—Y″-E I
wherein
A is
a group of formula 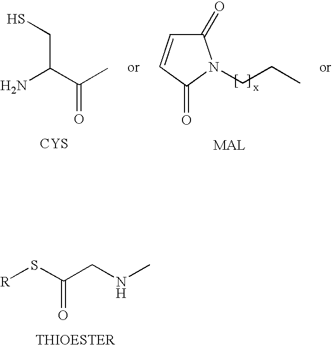
wherein x is 1 to 5, and
R is (C₁₋₄)alkyl carboxylic acid ester, e.g. propionic acid ethyl ester, or a group of formula 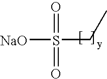
wherein y is 2 to 4, e.g. 2;
D is
a group of formula 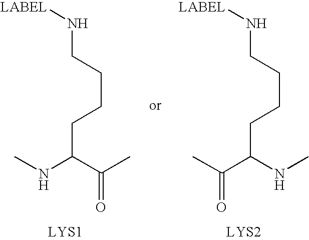
or a group of formula 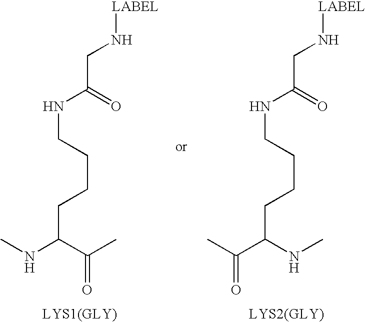
or a group of formula 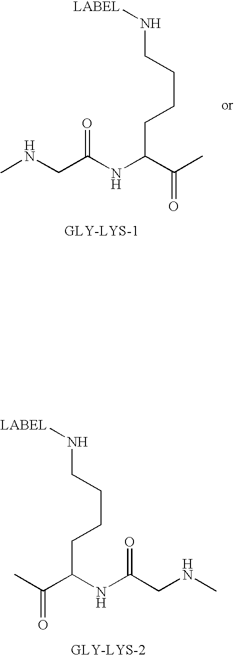
or a group of formula 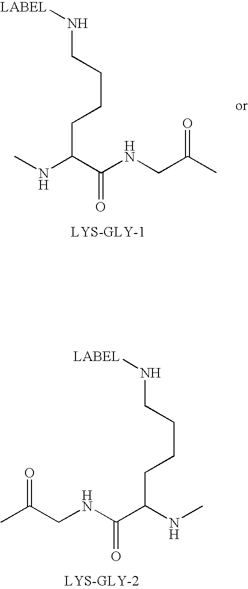
or a group of formula

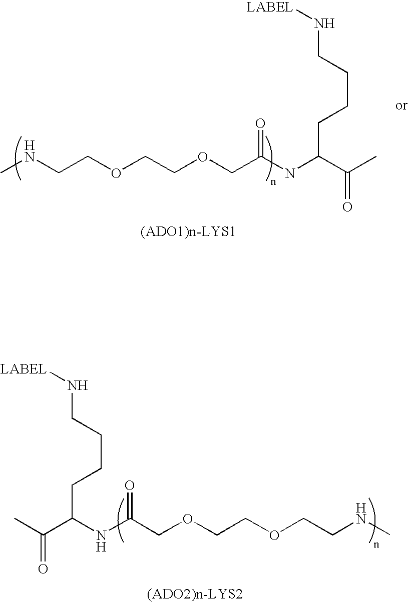

LABEL is a chelating ion or a dye, e.g. a fluorescent dye, such as a group of formula 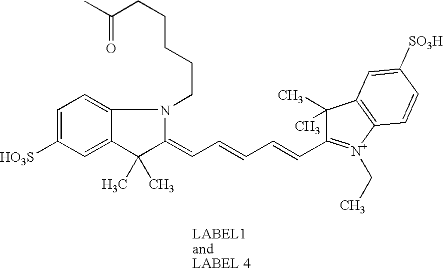
or a group of formula 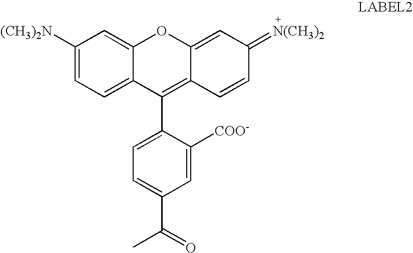
wherein ANION is a negatively charged group, e.g. an anion, such as CF₃COO⁻,
or a group of formula 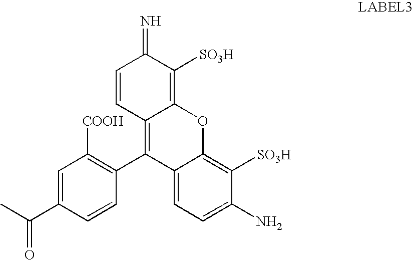
or a group of formula 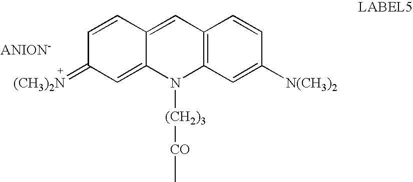
wherein ANION is a negatively charged group, e.g. an anion, such as CF₃COO⁻,
X is a group of formula 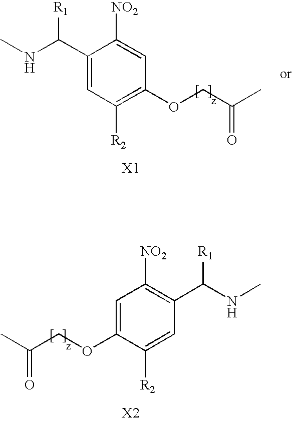
wherein
R₁ is H or CH₃, R₂ is H or —OCH₃ and z is 1 to 5;
E is a group of formula 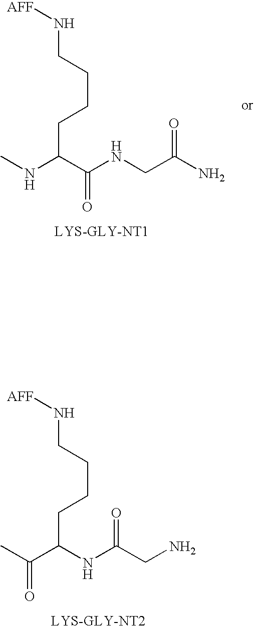
or a group of formula 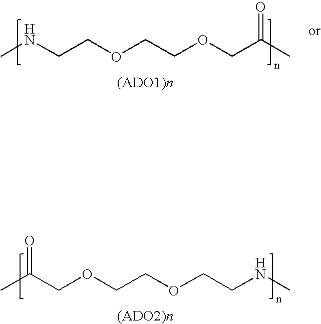
wherein to one of the amine groups a group AFF is bound and wherein n is 1 to 4, and
EITHER the affinity tag is bound to a carboxylic acid residue, e.g. the carboxylic residue of (ADO1)n and is a group of formula 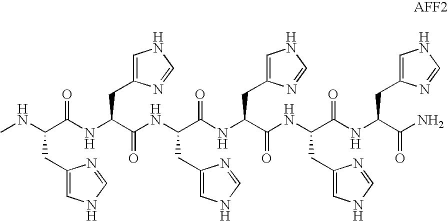
or of formula 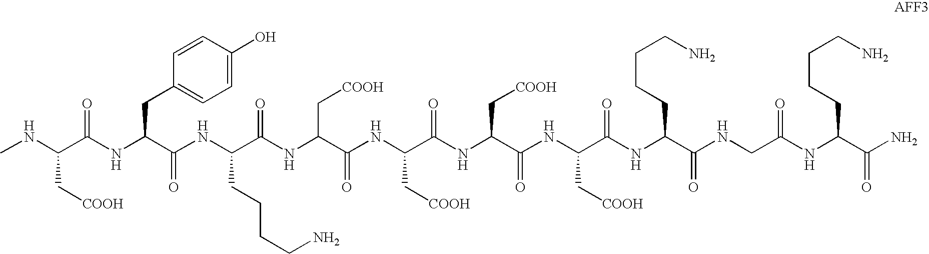
or of formula

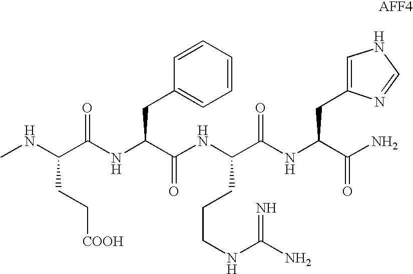

OR the affinity tag is bound to an amino group, e.g. to the ε-amino group of the lysine residue in LYS-GLY-NT1 or LYS-GLY-NT2; or to an amino group of (ADO1)n and is a group of formula 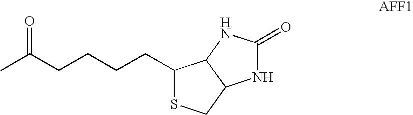
or of formula 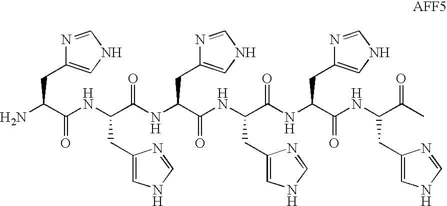
or of formula 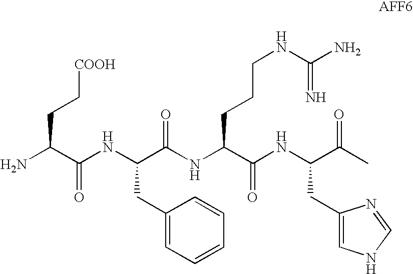
and
Y, Y′, Y″ independently of each other are a group of formula 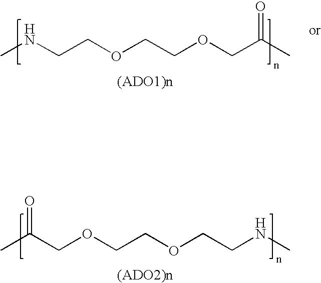
wherein n is 1 to 4,
or a group of formula 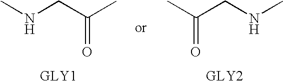
with the proviso, that

IF A is CYS or MAL,

THEN

D is LYS1, LYS1(GLY), GLY-LYS1, LYS-GLY1 or (ADO1)n-LYS1,

X is X1,

E is LYS-GLY-NT1 or (ADO1)n,

AFF is AFF1, AFF2, AFF3 or AFF4, and

Y, Y′ and Y″ independently of each other are GLY1 or (ADO1)n, wherein n is 1 to 4,

and

IF A is THIOESTER,

THEN

D is LYS2, GLY-LYS1, LYS-GLY2 or (ADO2)n-LYS1,

X is X2,

E is LYS-GLY-NT2 or (ADO2)n and n is 1 to 4,

AFF is AFF1, AFF5 or AFF6, and

Y, Y′ and Y″ independently of each other are GLY2 or (ADO2)n, wherein n is 1 to 4.

In another aspect the present invention provides a compound of the present invention, which is a compound of formula I, wherein A, Y, D, Y′, X, Y″ and E are as defined in TABLE 1 below, wherein in X1 and X2 R₁ is methyl, R₂ is methoxy and z=3, and in (ADO1)n and (ADO2)n n=1, except for EX 12 wherein n=3 and for EX 11, 15, and 26 to 29 wherein n=2.

TABLE 1

| EX | A | Y | D | LABEL | Y′ | X | Y″ | E | AFF |
|---|---|---|---|---|---|---|---|---|---|
| 8 | CYS | GLY1 | LYS1 | LABEL1 | GLY1 | X1 | ADO1 | LYS- | AFF1 |
| | | | | | | | | GLY- | |
| | | | | | | | | NT1 | |
| 9 | CYS | GLY1 | LYS1 | LABEL2 | GLY1 | X1 | ADO1 | LYS- | AFF1 |
| | | | | | | | | GLY- | |
| | | | | | | | | NT1 | |
| 10 | CYS | GLY1- | ADO1- | LABEL1 | GLY1 | X1 | ADO1 | LYS- | AFF1 |
| | | ADO1 | LYS1 | | | | | GLY- | |
| | | | | | | | | NT1 | |
| 11 | CYS | GLY1 | LYS1 | LABEL1 | GLY1 | X1 | (ADO1)₂ | LYS- | AFF1 |
| | | | | | | | | GLY- | |
| | | | | | | | | NT1 | |
| 12 | CYS | GLY1 | LYS1 | LABEL1 | GLY1 | X1 | (ADO1)₃ | LYS- | AFF1 |
| | | | | | | | | GLY- | |
| | | | | | | | | NT1 | |
| 13 | CYS | GLY1 | LYS1 | LABEL1 | GLY1 | X1 | — | ADO1 | AFF2 |
| 14 | CYS | GLY1 | LYS1 | LABEL1 | GLY1 | X1 | ADO1 | ADO1 | AFF2 |
| 15 | CYS | GLY1 | LYS1 | LABEL1 | GLY1 | X1 | (ADO1)₂ | ADO1 | AFF2 |
| 16 | CYS | GLY1— | LYS1 | LABEL2 | GLY1 | X1 | ADO1 | LYS- | AFF1 |
| | | GLY1 | (GLY) | | | | | GLY- | |
| | | | | | | | | NT1 | |
| 17 | CYS | GLY1 | LYS1 | LABEL1 | GLY1 | — | ADO1 | ADO1 | AFF3 |
| 18 | CYS | GLY1 | LYS1 | LABEL1 | GLY1 | X1 | — | ADO1 | AFF3 |
| 19 | CYS | GLY1 | LYS1 | LABEL1 | GLY1 | X1 | ADO1 | ADO1 | AFF3 |
| 20 | CYS | GLY1 | LYS1 | LABEL3 | GLY1 | X1 | ADO1 | ADO1 | AFF2 |
| 21 | CYS | GLY1 | LYS1 | LABEL4 | GLY1 | X1 | ADO1 | ADO1 | AFF4 |
| 22 | MAL | GLY1— | LYS1 | LABEL4 | GLY1 | X1 | ADO1 | ADO1 | AFF4 |
| | | GLY1 | | | | | | | |
| 23 | CYS | GLY1 | LYS1 | LABEL5 | GLY1 | X1 | ADO1 | ADO1 | AFF2 |
| 24 | CYS | GLY1 | LYS1 | LABEL3 | GLY1 | — | ADO1 | ADO1 | AFF2 |
| 25 | CYS | GLY1 | LYS1 | LABEL1 | GLY1 | — | ADO1 | ADO1 | AFF2 |
| 26 | THIOESTER; | GLY2 | LYS2 | LABEL1 | GLY2 | X2 | (ADO2)₂ | LYS- | AFF1 |
| | R = (CH₂)₂—CO— | | | | | | | GLY- | |
| | OEt | | | | | | | NT2 | |
| 27 | THIOESTER | GLY2 | LYS2 | LABEL5 | GLY2 | X2 | (ADO2)₂ | LYS- | AFF1 |
| | R = (CH₂)₂—CO— | | | | | | | GLY- | |
| | OEt | | | | | | | NT2 | |
| 28 | THIOESTER | GLY2 | LYS2 | LABEL1 | GLY2 | X2 | (ADO2)₂ | LYS- | AFF1 |
| | R = (CH₂)₂—SO₃H | | | | | | | GLY- | |
| | | | | | | | | NT2 | |
| 29 | THIOESTER | GLY2 | LYS2 | LABEL5 | GLY2 | X2 | (ADO2)₂ | LYS- | AFF1 |
| | R = (CH₂)₂—SO₃H | | | | | | | GLY- | |
| | | | | | | | | NT2 | |
| 30 | THIOESTER | GLY2 | LYS2 | LABEL5 | GLY2 | X2 | ADO2 | ADO2 | AFF5 |
| | R = (CH₂)₂—CO— | | | | | | | | |
| | OEt | | | | | | | | |
| 31 | THIOESTER | GLY2 | LYS2 | LABEL1 | GLY2 | X2 | ADO2 | ADO2 | AFF5 |
| | R = (CH₂)₂—CO— | | | | | | | | |
| | OEt | | | | | | | | |
| 32 | THIOESTER | GLY2 | LYS2 | LABEL1 | GLY2 | X2 | ADO2 | ADO2 | AFF6 |
| | R = (CH₂)₂—SO₃H | | | | | | | | |
| 33 | MAL | GLY1 | GLY- | LABEL2 | GLY1 | X1 | ADO1 | LYS- | AFF1 |
| | | | LYS1 | | | | | GLY- | |
| | | | | | | | | NT1 | |

In TABLE 1 the groups

CYS, MAL, THIOESTER, GLY1, GLY2, LYS1, LYS1(GLY), LYS2, LABEL1, LABEL2, LABEL3, LABEL4, LABEL5, X1, X2, (ADO1)n, (ADO2)n, LYS-GLY-NT1, LYS-GLY-NT2, AFF1, AFF2, AFF3, AFF4, AFF5 and AFF6

are groups as defined above in the corresponding formulae. EX is the examples number (correlating to the examples as set out herein).

ADO1 is the group (ADO1)n, wherein n is 1 and ADO2 is the group (ADO2)n, wherein n is 1. GLY1-GLY1 are two residues GLY1 covalently bound to each other via an amide bond. GLY2-GLY2 are two residues GLY1 covalently bound to each other via an amide bond.

In another aspect the present invention provides a compound of formula 1, which is selected from the group consisting of compounds of formulae
CYS-GLY1-LYS1 (LABEL1)-GLY1-X1-ADO1-LYS1 (AFF1)-GLY-NT1;
CYS-GLY1-LYS1 (LABEL2)-GLY1-X1-ADO1-LYS1 (AFF1)-GLY-NT1;
CYS-GLY1-ADO1-ADO1-LYS1 (LABEL1)-GLY1-X1-ADO1-LYS1 (AFF1)-GLY-NT1;
CYS-GLY1-LYS1 (LABEL1)-GLY1-X1-ADO1-ADO1-LYS1 (AFF1)GLY-NT1;
CYS-GLY1-LYS1 (LABEL1)-GLY1-X1-ADO1-ADO1-ADO1-LYS1 (AFF1)-GLY-NT1;
CYS-GLY1-LYS1 (LABEL1)-GLY1-X1-ADO1(AFF2);
CYS-GLY1-LYS1 (LABEL1)-GLY1-X1-ADO1-ADO1 (AFF2);
CYS-GLY1-LYS1 (LABEL1)-GLY1-X1-ADO1-ADO1-ADO1 (AFF2);
CYS-GLY1-GLY1-LYS1 (GLY)(LABEL2)-GLY1-X1-ADO1-LYS1 (AFF1)-GLY-NT1;
CYS-GLY1-LYS1 (LABEL1)-GLY1-ADO1-ADO1 (AFF3);
CYS-GLY1-LYS1 (LABEL1)-GLY1-X1-ADO1 (AFF3);
CYS-GLY1-LYS1 (LABEL1)-GLY1-X1-ADO1-ADO1 (AFF3);
CYS-GLY1-LYS1 (LABEL3)-GLY1-X1-ADO1-ADO1 (AFF2);
CYS-GLY1-LYS1 (LABEL4)-GLY1-X1-ADO1-ADO1 (AFF4);
MAL-GLY1-GLY1-LYS1 (LABEL4)-GLY1-X1-ADO1-ADO1 (AFF4);
CYS-GLY1-LYS1 (LABEL5)-GLY1-X1-ADO1-ADO1 (AFF2);
CYS-GLY1-LYS1 (LABEL3)-GLY1-ADO1-ADO1 (AFF2);
CYS-GLY1-LYS1 (LABEL1)-GLY1-ADO1-ADO1 (AFF2);
THIOESTER-GLY2-LYS2(LABEL1)-GLY2-X2-ADO2-ADO2-LYS2(AFF1)-GLY-NT2; wherein R is (CH₂)₂—CO-OEt,
THIOESTER-GLY2-LYS2(LABEL5)-GLY2-X2-ADO2-ADO2-LYS2(AFF1)-GLY-NT2; wherein R is (CH₂)₂—CO-OEt,
THIOESTER-GLY2-LYS2(LABEL1)-GLY2-X2-ADO2-ADO2-LYS2(AFF1)-GLY-NT2; wherein R is (CH₂)₂—SO₃H or (CH₂)₂SO₃Na,
THIOESTER-GLY2-LYS2(LABEL5)-GLY2-X2-ADO2-ADO2-LYS2(AFF1)-GLY-NT2, wherein R is (CH₂)₂—SO₃H or (CH₂)₂—SO₃Na,
THIOESTER-GLY2-LYS2(LABEL5)-GLY2-X2-ADO2-ADO2(AFF5); wherein R is (CH₂)₂—CO-OEt
THIOESTER-GLY2-LYS2(LABEL1)-GLY2-X2-ADO2-ADO2(AFF5); wherein R is (CH₂)₂—CO-OEt
THIOESTER-GLY2-LYS2(LABEL1)-GLY2-X2-ADO2-ADO2(AFF6), wherein R is (CH₂)₂—SO₃H or (CH₂)₂—SO₃Na, and
MAL-GLY1-GLY1-LYS1 (LABEL2)-GLY1-X1-ADO1-LYS1 (AFF1)-GLY-GLY-NT1; wherein the groups
   CYS, MAL, THIOESTER, GLY1, GLY2, LYS1, LYS2, LABEL1, LABEL2, LABEL3, LABEL4, LABEL5, X1, X2, (ADO1)n, (ADO2)n, LYS-GLY-NT1, LYS-GLY-NT2, AFF1, AFF2, AFF3, AFF4, AFF5 and AFF6 are as defined above in the corresponding formulae. ADO1 is the group (ADO1)n, wherein n is 1 and ADO2 is the group (ADO2)n, wherein n is 1.

In a compound of the present invention each single defined substitutent (group) may be a preferred substituent (group), e.g. independently of each other substitutent (group) defined. A compound of the present invention includes a compound of formula 1. A compound of the present invention includes a compound in any form, e.g. in free form, in the form of a salt, in the form of a solvate and in the form of a salt and a solvate.

In a compound of the present invention each single defined substitutent (group) may be a preferred substituent (group), e.g. independently of each other substitutent (group) defined.

In another aspect the present invention provides a compound of the present invention in the form of a salt.

A salt of a compound of the present invention includes a metal salt, an acid addition salt, or an inner salt, e.g. a quaternary salt. An inner salt includes a salt of a positively charged nitrogen atom, e.g. a quaternary ammonium salt or a quaternary iminium salt, with the anion of an acid, such as a carboxylic acid or a sulfonic acid as the counter ion. Metal salts include for example alkali or earth alkali salts; acid addition salts include salts of a compound of formula I with an acid, e.g. fumaric acid, naphthalin-1,5-sulphonic acid, hydrochloric acid, deuterochloric acid, trifluoroacetic acid; preferably trifluoroacetic acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of pure isomers or mixtures thereof; e.g. optical isomers, diastereoisomers, cis/trans isomers. A compound of the present invention may e.g. contain asymmetric carbon atoms and may thus exist in the form of enantiomers or diastereoisomers and mixtures thereof, e.g. racemates. A substituent attached to an asymmetric carbon atom may be present in the (R)-, (S)- or (R,S)-configuration, preferably in the (R)- or (S)-configuration.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

A compound of the present invention may be prepared as appropriate, e.g. according, e.g. analogously, to a method as conventional, e.g. according to SCHEME 1 or SCHEME 2 as indicated below.

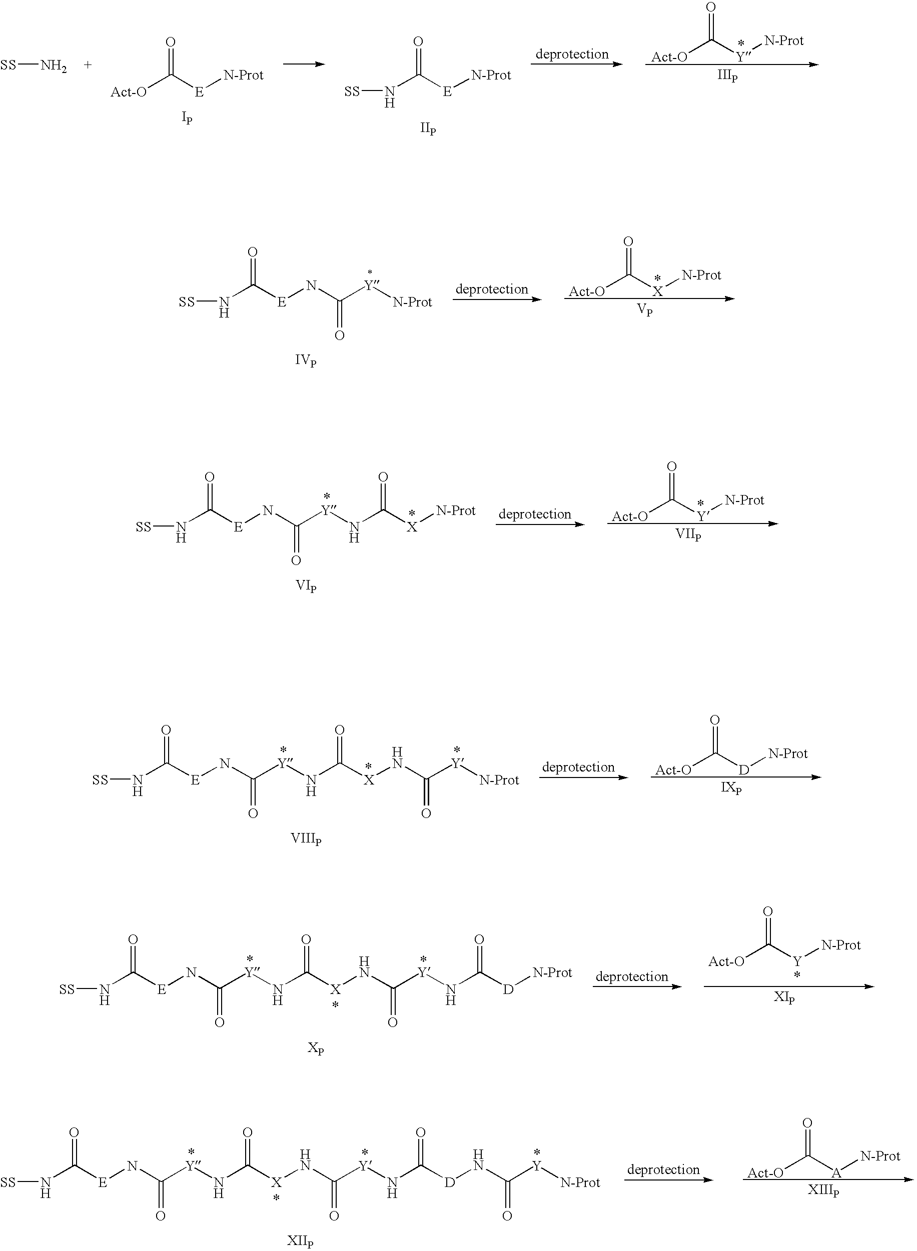

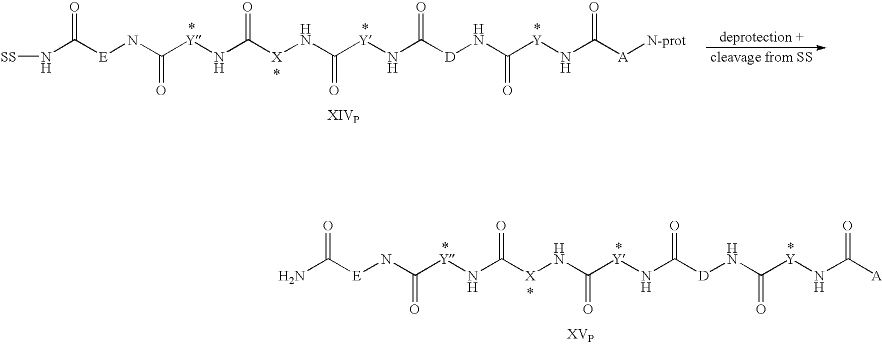

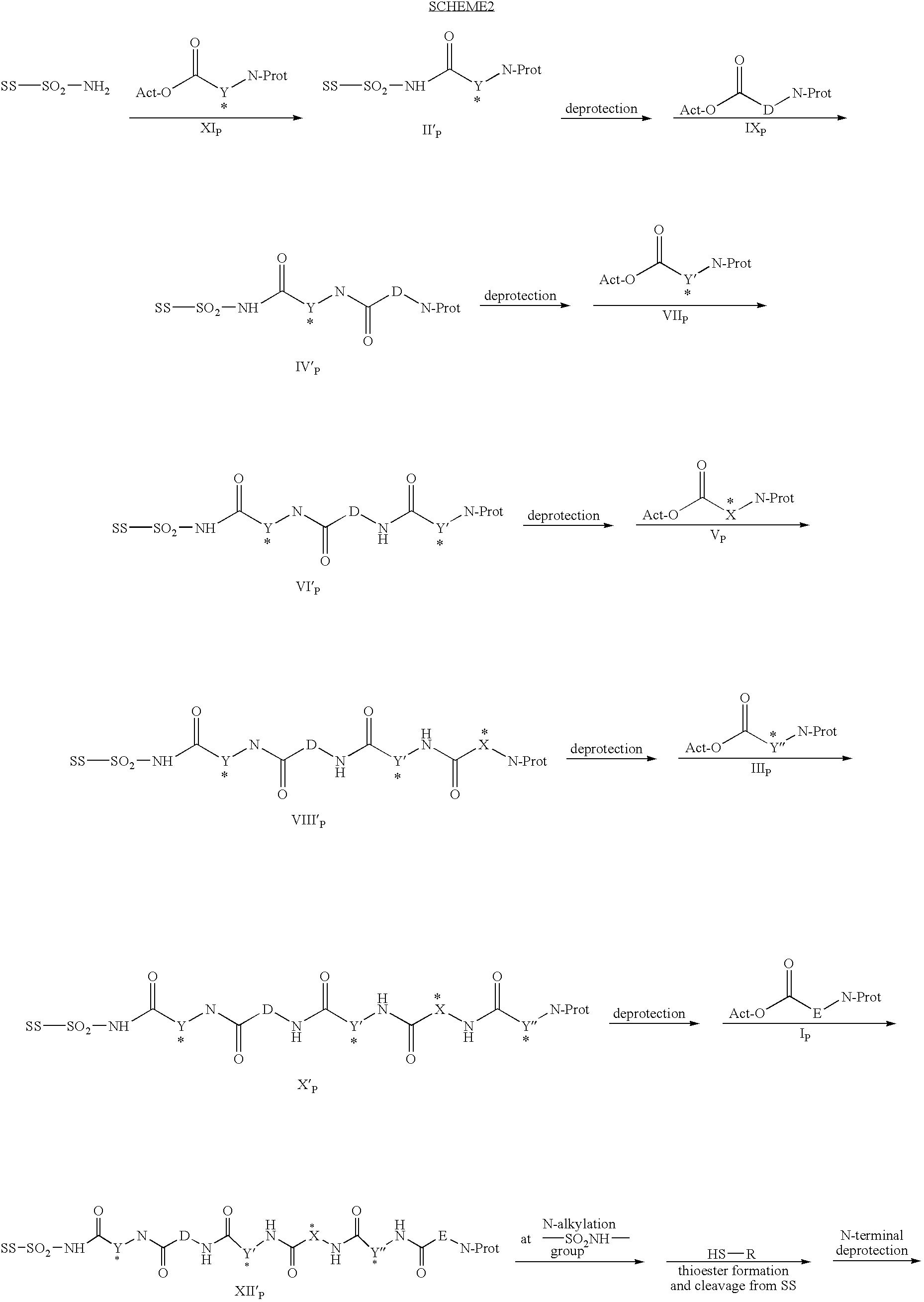

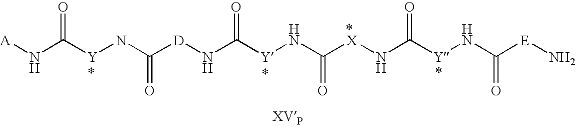

The preparation of a compound of the present invention is preferably carried out on solid support SS. An appropriate solid support is known, or may be provided as appropriate, e.g. according, e.g. analogously, to a method as conventional.

In SCHEME 1 and SCHEME 2 “Prot” is a protecting group, and “Act” means that the carboxylic function to which Act is attached, is in an activated form, e.g. in the form of an ester. R is as a residue of a carboxylic acid derivative, e.g. as defined in a compound of the present invention. The groups A, D, E, Y, Y′, Y″ and X are as defined in a compound of the present invention.

In SCHEME 1 and SCHEME 2, the solid support is such, that it has an end-positioned functional group attached, conveniently via a spacer. Preferably SS has end-positioned amino groups attached. SS includes an appropriate polymer, such as a polystyrol, as a backbone material, optionally containing spacers, e.g. PEG, and having covalently bound a group with an end-positioned amino group, such as Tentagel-S-RAM™ resin. An SS may also comprise a —SO₂— group bound to an end-positioned amino group, e.g. such as a 4-sulfamylbutyryl group, e.g. present on a resin as described above.

An end-positioned amino group of SS I_{P} reacted with a compound of formula I_{P} under appropriate reaction conditions so that an amide bond is formed between said amino group and the activated carboxylic acid residue of compound I_{P}. Activation of the carboxylic acid residue to obtain a group Act-O—CO— may be performed as appropriate, e.g. by a methods as conventional, e.g. by converting the carboxylic acid function into an ester, e.g. an N-hydroxybenzotriazolester. The amino acid in compound I_{P} or II_{P} is protected by an N-protective group, including protection groups as conventional, e.g. Fmoc. A compound of formula II_{P} may be deprotected as appropriate, e.g. by a method as conventional, such as via treatment with 20% piperidine in DMF if the protecting group is an Fmoc-group. Further functional groups which may be present in any compound of SCHEME 1 and SCHEME 2 may be protected appropriately, e.g. according to methods as conventional in peptide synthesis. Further amide formation, deprotection and activation reactions may be performed at appropriate conditions, e.g. similar to those described above.

Reactions involving compounds marked with a star (*), are independently of each other optional, e.g. such reactions are only carried out, if a group Y, Y′, Y″ and/or X is desired in a compound of formula XV_{P} of SCHEME 1, or XV′_{P} of SCHEME 2, respectively.

In SCHEME 1 or in SCHEME 2, in a group “D”, a label may be already part of a compound of formula IX_{P} or such label may be introduced at an appropriate stage, e.g. before deprotection and cleavage from SS. Similarly, an affinity tagging group may be already part of compound I_{P} or may be introduced at an appropriate stage, e.g. before cleavage from SS. Cleavage from SS may be carried out as appropriate, e.g. according, e.g. analogously, to a method as conventional. A compound of formula XV_{P} or XV′_{P}, which are compounds of the present invention, are obtained.

In any of the compounds of SCHEME 1 and SCHEME 2, functional groups, if present, optionally may be in protected form or in the form of a salt, if a salt-forming group is present. Protecting groups, optionally present, may be removed at an appropriate stage, e.g. according, e.g. analogously, to a method as conventional.

The above reaction is a peptide synthesis reaction, preferably a solid phase peptide synthesis (reactions on solid support), and may be carried out as appropriate, e.g. according, e.g. analogously, to a method as conventional.

Any compound obtained according to SCHEME 1 and SCHEME, e.g. to its fixation on a solid support, may be easily purified, e.g. impurities present in the surrounding solvent (system) after individual reactions can easily be removed at any stage of such process, e.g. by change of the solvent (system) and washing.

A compound of the present invention thus obtained may be converted into another compound of the present invention, e.g. or a compound of the present invention obtained in free form may be converted into a salt of a compound of the present invention and vice versa.

In another aspect the present invention provides a compound of the present invention bound to a solid support via a terminal functional group, such as an amine group, e.g. a compound of formula XIV or XIV′ of SCHEME 1 or SCHEME 2.

Such terminal functional group is preferably bound to an amino acid or spacer bound to said solid support.

Any compound described herein, e.g. a compound of the present invention, a molecule of the present invention, a molecule of the present invention bound to an affinity support, a compound of the present invention bound to a solid support and compounds of formulae I, I_{P}, II_{P}, II′_{P}, III_{P}, IV_{P}, IV_{P}, V_{P}, VI_{P}, VI′_{P}, VII_{P}, VIII_{P}, VIII′_{P}, IX_{P}, X_{P}, X′_{P}, XI_{P}, XII_{P}, XII′_{P}, XIII_{P} and XIV may be prepared as appropriate, e.g. according, e.g. analogously, to a method as conventional, e.g. or as specified herein.

The present invention provides a useful method for the specific binding of a target protein or target peptide to a compound of the present invention, and thus allows exact positioning of the label and simple separation of unreacted target protein or target peptide and unreacted labeling reagent from labeled target protein or labeled target peptide.

E.g. according to a method of the present invention it is possible to obtain a target protein or target peptide 1:1 labeled, e.g. if
the target protein or target peptide has one N-terminal cysteine residue and the reactive group containing residue in a compound of the present invention includes a thioester function, or
the target protein or target peptide has one internal cysteine residue and the reactive group containing residue in a compound of the present invention is a thiol-specific group, e.g. a iodoacetyl or a maleimido group.

In a molecule of the present invention a labeled target protein or target peptide may be separated from unlabeled target protein or target peptide due to the presence of the affinity tagging group via the affinity support to which a labeled target protein or target peptide is bound whereas the unlabeled target protein or target peptide is not. Thus, according to a method of the present invention pure labeled target protein or target peptide may be obtained as the product

In another aspect the present invention provides
the use of a compound of the present invention for the labeling of a target protein or target peptide;
the use of a compound of the present invention for generating a highly pure, e.g. 1:1 labeled, target protein or target peptide.

A labeled target protein or target peptide obtained according to a method of the present invention is a useful tool in high throughput screening (HTS), e.g. for identifying an agent that modulates the activity or characteristic of a target protein.

In another aspect the present invention provides the use of a target protein or target peptide bound to a compound of the present invention via its reactive group, in a high throughput screening assay.

In a further aspect the present invention provides a kit comprising a compound of the present invention, or a compound of the present invention bound via its reactive group to a specific target protein or target peptide, and instructions for using the kit.

Such kit may further comprise a substantial component including an appropriate environment of a sample to be tested and, e.g. appropriate means to determine the effect of a candidate compound in a sample to be tested.

In another aspect the present invention provides a method for identifying an agent that modulates the activity or characteristic of a target protein or a target peptide comprising the steps
a. contacting a target protein or target peptide bound to a compound of the present invention, with a candidate compound, which is expected to modulate the activity or characteristics of said target protein or target peptide,
b. measuring a signal,
b1. in the absence of such candidate compound, and
b2. in the presence of such candidate compound and determining whether there is a difference in the signals measured in steps b1. and b2., and
c. choosing an agent determined in step b., e.g. for use as a pharmaceutical.

A signal includes a signal originating from an enzymatic reaction, magnetic resonance or fluorescence, e.g. originating from the labeling residue in a molecule of the present invention, wherein the label is a fluorescent dye or chelated ion.

The measurement of such signal may be carried out with a spectrofluorometer or other known optical fluorescence detection methods with a particular focus on confocal fluctuation detection with single molecule sensitivity auch as e.g. Fluorescence Correlation Spectroscopy (FCS), Fluorescence Intensity Distribution Analysis (FIDA), or applications based on the determination of Fluorescence Anisotropy or Fluorescence Resonance Energy Transfer (FRET) as well as multiplexed techniques such as Fluorescence Intensity Multiple Distribution Analysis (FIMDA), 2 dimensional 2 colour Fluorescence Intensity Distribution Analysis (2D-FIDA) or Multidimensional Fluorescence Detection (MFD) as well as fluorescent detection using confocal imaging applications.

Alternatively, such a signal may be obtained using a magnetic resonance detection device, e.g. as used in magnetic resonance imaging.

A typical experimental setup for identifying a candidate compound may be as follows: A candidate compound library is provided, e.g. covalently bound on beads, which beads may be located on a plate (e.g. a 96 well plate) and the labeled target protein or target peptide bound to a compound of the present invention is contacted with said beads. In case said labeled protein or labeled peptide binds to a bead having bound a candidate compound from said candidate compound library, a signal, e.g. a signal originating from fluorescence, can be measured in using e.g. confocal fluorescence detection and an agent which modulates said target protein or target peptide can be identified.

A candidate compound includes compound(s)(libraries) from which its influence on the target protein or target peptide is unknown. Compound (libraries) include for example oligopeptides, polypeptides, proteins, antibodies, mimetics, small molecules, e.g. low molecular weight compounds (LMW's).

An agent is understood to be a candidate compound from which effective modulation of said target protein, e.g. inhibition or enhancement of a biological activity of such target protein or target peptide, is determined in a method for identification according to the present invention. An agent is one of the candidate compounds chosen in step c.

An agent of the present invention may exhibit pharmacological activity and is expected to be useful as a pharmaceutical.

For pharmaceutical use an agent of the present invention for treatment includes one or more, preferably one, agent of the present invention, e.g. a combination of two or more agents of the present invention.

For such treatment, the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 1.0 g, of an agent of the present invention; conveniently administered, for example, in divided doses up to four times a day.

An agent of the present invention may be administered by any conventional route, for example enterally, e.g. including nasal, buccal, rectal, oral administration; parenterally, e.g. including intravenous, intramuscular, subcutanous administration; or topically; e.g. including epicutaneous, intranasal, intratracheal administration;

e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition comprising an agent identified in a method of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such compositions may be manufactured according, e.g. analogously, to a method as conventional, e.g. by mixing, granulating, coating, dissolving or lyophilizing processes. Unit dosage forms may contain, for example, from about 0.5 mg to about 2000 mg, such as 1 mg to about 500 mg, e.g. 0.00625 mg/kg to about 12.5 mg/kg.

In another aspect the present invention provides
an agent identified by a method according to the present invention for use as a pharmaceutical;
the use of an agent identified by a method according to the present invention, for the manufacture of a medicament, e.g. a pharmaceutical composition, for the treatment of a disorder, which disorder is mediated by a target protein or target peptide in vivo;
a method of treatment disorders mediated by a target protein or target peptide in vivo, comprising administering to a subject in need of such treatment an effective amount of an agent identified by a method according to the present invention.

In another aspect the present invention provides a compound of formula
A-Y-D-Y′—X—Y″-E I_{S}
wherein
A comprises a chemical reactive group,
D is a label bearing residue,
X is present and is a linker residue allowing cleavage of compounds of formula I to liberate A-Y-D-Y′ fragment with Y and/or Y′ being present or not, respectively, or X is not present,
Y and/or Y′ and/or Y″ is present and independently of each other is a spacer residue or Y and/or Y′and/or Y″ is not present, and
E is an affinity tag residue.

### DESCRIPTION OF THE FIGURES

FIG. 1: A specifically applied design of a compound of formula I comprising as the reactive group (A) either an N-terminal cysteine or a thioester or a maleimido-, iodoacetyl- or vinylsulfone-function, a branchpoint for a LABEL (e.g. a dye), e.g. a lysine branchpoint, attachment (peptide=D), a cleavable linker (X) and an affinity tag (E), e.g. Biotin, His₆, FLAG, APP. The compound can further contain one or more hydrophilic spacer residue(s) of variable length (e.g. 8-amino-3,6-oxaoctanoic acid).

FIG. 2: LC/ESI-MS of the purified compound of example 1 bound to HuR12.

FIG. 3: Schematic representation of the labeling procedure according to the present invention and purification of the labeled protein (peptide). The affinity tag is an irreversible affinity tag and remains bound to the affinity support after (photo)cleavage of the (photo)linker (FIG. 3A) or the affinity tag is a reversible affinity tag and is eluted from the affinity support before (photo)cleavage of the (photo)linker is carried out (FIG. 3B) or the affinity tag is an irreversible affinity tag and a prepurification, e.g. size exclusion chromatography, is carried out before affinity purification (FIG. 3C) or the affinity tag is a reversible affinity tag and a prepurification, e.g. size exclusion chromatography, is carried out before affinity purification (FIG. 3D).

In the following examples all temperatures are in degree centigrade (° C.) and are uncorrected. RT means room temperatures.

The following ABBREVIATIONS are used:
ADO spacer 8-amino-3,6-dioxaoctanoic acid (Neosystem)
Alexa488-maleimide reactive maleimide derivative of fluorescent dye Alexa488™ (Molecular Probes)
APP EFRH (amino acid sequence in 1 letter code), binding epitope of α-APP (amyloid precursor protein) antibody
Atto495-COOH carboxylic acid derivative of fluorescent acridine dye Atto495™ (AttoTec, Germany)
Atto495NHS reactive N-hydroxy-succinimidyl ester derivative of fluorescent acridine dye Atto495™ (AttoTec, Germany)
Boc t-butyloxycarbonyl-
Cys cysteine
Cy5-maleimide reactive maleimide derivative of fluorescent merocyanine dye Cy5™ (Amersham Pharmacia Biotech)
Cy5-NHS reactive N-hydroxy-succinimidyl ester derivative of fluorescent merocyanine dye Cy5™ (Amersham Pharmacia Biotech)
Dde (4,4-dimethyl-2,6-dioxocyclohex-1-ylidene)ethyl-
DTT dithiothreitol
DIC N,N′-diisopropylcarbodiimide
DIPEA diisopropylethylamine EDT ethandithiol
ESI-MS electrospray injection spectrometry
FF fast flow
FLAG DYKDDDDKGK amino acid sequence in one letter code
Fmoc Fluorenylmethoxycarbonyl
His₆ hexa-histidine
HOBt 1-hydroxybenzotriazol
HTS high throughput screening
HuR Hu antigen R (antigen present in patients with Hu-syndrome)
Hu-syndrome paraneoplastic encephalomyelitis sensory neuropathy
HuR12 variant of HuR antigen
ivDde 1-(4,4-dimethyl-2,6-dioxocyclohex-1-ylidene)-3-methylbutyl-
LC/ESI MS liquid chromatography/electrospray injection mass spectrometry
MALDI-MS matrix assisted laser desorption injection mass spectrometry
2-MESNA 2-mercaptoethanesulfonic acid sodium salt
Mtr 4-methyltriphenylmethyl-
MPE 3-mercaptopropionic acid ethyl ester
NTA nitrilotriacetic acid
PBS phosphate buffered saline
PyBop (benzotriazol-1-yloxy)-tripyrrolidinophosphonium-hexafluorophosphate
RAM Rink Amide (type of resin esp. useful in solid phase techniques)
RP-HPLC reversed phase high performance liquid chromatography
sh shoulder
TBTU O-(benzotriazol-1-yl)-N,N,N′,N′-tetramethyluroniumtetra-fluoroborate
tBu tert.butyl-
TCEP Tris(2-carboxyethyl)phosphine hydrochloride
THF tetrahydrofuran
TMR-maleimide reactive maleimide derivative of fluorescent dye tetramethylrhodamine (Molecular Probes)
TMR-COOH carboxylic acid derivative of fluorescent dye tetramethylrhodamine (Molecular Probes)
TMR-NHS reactive N-hydroxy-succinimidyl ester derivative of fluorescent dye tetramethylrhodamine (Molecular Probes)
Trt triphenylmethyl-
TSTU O-(N-Succinimidyl)-N,N,N′,N′-tetramethyluroniumtetrafluoroborate

### EXAMPLE 1

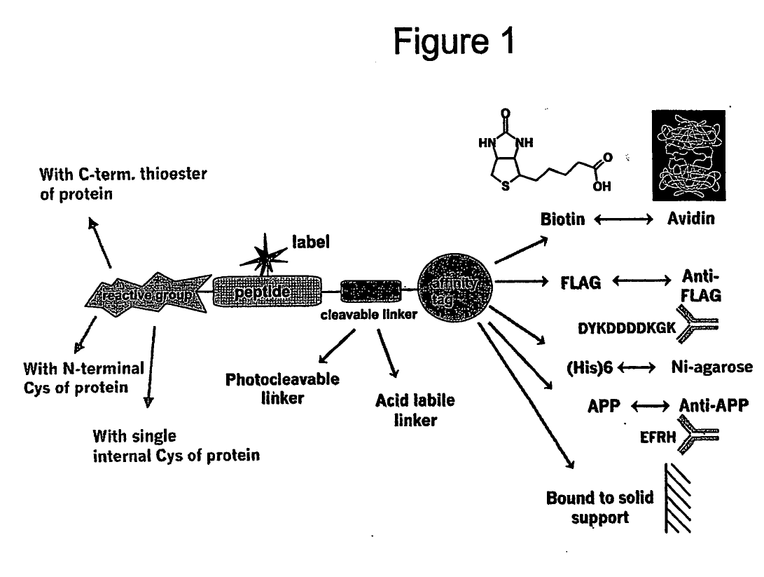

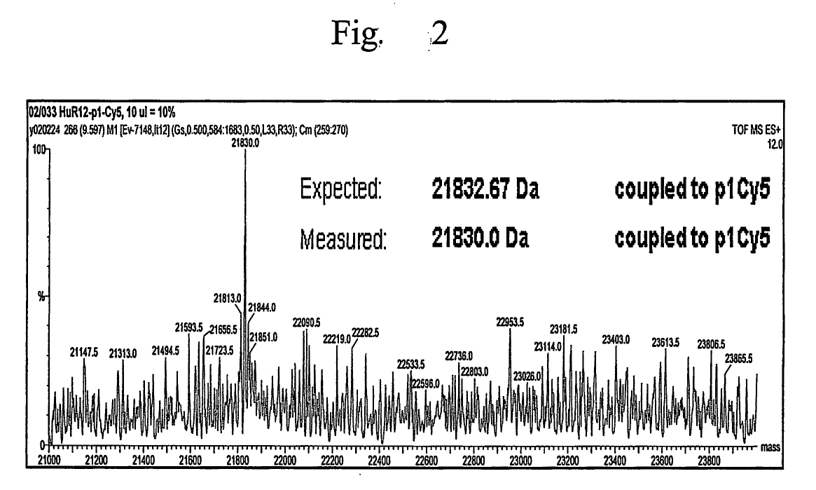

Compounds of Formula I (=Peptide Reagent; see e.g. FIG. 1).

In the following examples a compound of formula I, e.g. a peptide reagent, is defined as a compound (also designated construct) comprising A, D and E, optionally a linker residue X and optionally spacer residues Y and/or Y′ and/or Y″. Suitable reagents (as given in e.g. examples 8 to 33, also without variable spacer residue(s)) with peptidic backbone as the label containing residue D are prepared by stepwise synthesis on solid support (e.g. Tentagel-S-RAM-resin) using general peptide coupling chemistry (e.g. Fmoc peptide chemistry) with DIC/HOBt, TBTU/DIPEA, TSTU/DIPEA activation or other activation procedures as conventional and protected or modified amino acids (see e.g. Chan W. and White P. D., Basic procedures: Fmoc solid phase synthesis, A practical approach, 41-76, 200, New York, Oxford University Press). Protected aminoalkoxycarboxylic acids or e.g. aminoalkyl-nitroaryl-alkoxycarboxylic acids are optionally introduced as additional spacer residues Y, Y′and/or Y″ or as cleavable linker residue X. Selective deprotection, e.g. of E-amino groups of lysines, allows introduction of fluorescent dyes (e.g. fluorescein, tetramethylrhodamine, merocyanines, acridines, ALEXA™, Bodipy™) as carboxylic acid with PyBop/DI PEA activation or other known activation procedures or as activated esters into side chains, e.g. lysines. A crude compound of formula I (peptide reagent) is obtained by cleavage from the solid support, on which the compound is synthesized and removal of the remaining protective groups. The crude compound or reagent can be further purified, e.g. chromatographically.

1.1. Labeled peptide reagents for native ligation with the C-terminal thioester of a target protein or target peptide are prepared on solid support, e.g. Rapp Tentagel-S-RAM™ or Polystyrene-RAM, with acid-labile Rink amide groups, using N-Fmoc protected amino acid or aminoalkylcarboxylic acid building blocks with acid-labile side chain protection if appropriate e.g. tBu-protection for hydroxyl functions, tBu-protection for carboxylic acid functions, Boc-protection for amino groups or trityl-protection for secondary amine function in an imidazole residue of histidine. Only the s-amino group of lysine building blocks used for attachment of dye labels is protected with hydrazine-labile Dde- or ivDde protection groups. The biotin label is introduced as ε-amino-derivative of L-lysine, hydrophilic spacers as N-Fmoc-8-amino-3,6-dioxaoctanoic acid and the photolabile linker as 4-[4{1-(9-fluorenylmethoxycarbonylamino)ethyl}-2-methoxy-5-nitrophenoxy]butanoic acid. For the N-terminal cysteine N-Boc-S-trityl-protection is selected. All reaction steps, cleavages, work up and purification during and after photolinker incorporation are performed under protection against direct light.

The Fmoc-protection group of the resin is removed by treating 3 times with 2-3 resin bed volumes of 20% piperidine/DMF for 20 minutes each, followed by washing 6 times with DMF. The first N-Fmoc protected amino acid or aminoalkylcarboxylic acid building block is coupled by dissolving 4 eq. protected amino acid or aminocarboxylic acid and 4 eq. of HOBt in anhydrous DMF to give a 0.2 M solution, 4 eq. of DIC are added and pre-activation is carried out for 10 minutes, resin is added and agitation is carried out for 10-14 hours at ambient temperature. The resin obtained is drained and washed 6 times with DMF, 3 times with MeOH and 3 times with CH₂Cl₂. The complete backbone of the peptide reagent is prepared by repetitive cycles of Fmoc-deprotection and coupling with the appropriate protected building blocks.

Alternatively the coupling procedure is performed with 3 eq. of N-Fmoc-protected amino acid, 3 eq. of TBTU and 3 eq. of HOBt, dissolved in DMF at 0.2 M concentration, 6 eq. of DIPEA are added, resin is added and agitation is carried out for 12-16 hours at 4°. Protected histidines (4 eq.) are coupled with 4 eq. of HBTU and 8 eq. of DIPEA for 12-16 hours at 4°. For selective deprotection of the Dde- or ivDde-group at the ε-amine of lysine and introduction of the dye label, the resin obtained is washed 2 times with DMF and agitated 3 times with 3 bed volumes of a 3-5% solution of hydrazine in DMF (made from hydrazine monohydrate >99%) for 10 minutes each. The resin obtained is drained and washed 5×5 min with DMF absolute and drained and flushed with argon.

1.5 eq. of the dye in form of its carboxylic acid is dissolved in anhydrous DMF to obtain a 0.15-0.2 M solution. The solution obtained is transferred to the resin, 3 eq. of DIPEA are added, mixed and cooled for 10 minutes at 4°. 1.5 eq. of solid PyBOP is added, mixed and the vial is agitated on a rotor at 4° for 7-14 hours. The resin obtained is drained, washed 6-8× with DMF until no dye is eluted from the resin. washed with MeOH and DCM and dried. Alternatively the resin is reacted with 1.5 eq. of the dye in form of its carboxylic acid N-hydroxysuccinimidyl ester in anhydrous DMF for 16 hours at RT.

For tags with a glycine spacer between the ε-amino group and the dye, the deprotected ε-amine on the resin is first coupled with 4 eq. of N-Fmoc-glycine, 4 eq. of HOBt and 4 eq. of DIC and deprotected with 20% piperidine/DMF, as described above, then coupled with the dye as described.

The cleavage of the peptide reagent from the resin and the simultaneous cleavage of the acid-labile protection groups is performed for reagents with biotin and FLAG affinity tags by two treatments of about 1 hour each in polypropylene syringes with frit and stopcock, occasional under agitation, with a surplus of about 3 bed volumes TFA/DCM/TIS/H2O/EDT 50:45:2:2.5:2.5 (v/v). TFA mixture washings and DCM washings are drained into a flask, in some instances the resins are treated additionally with TFA/DCM/TIS/H2O/EDT 95:5:2:2.5:2.5 (v/v) for 20 minutes. The solutions obtained are concentrated and co-evaporated with DCM and with MeOH. The resins obtained are additional treated with MeOH and with H₂O for 30 minutes and solvent is evaporated. The cleaved residue is triturated 3 times with diethylether under argon, the residue obtained is dissolved in a small amount of H₂O and vortexed with a large surplus of diethylether to precipitate the dye-labeled compound. After centrifugation supernatant is removed, further ether washings are carried out and the residue obtained is dried. The compound obtained is dissolved in DMF (about 20 μl/mg) and optionally further purified by HPLC, e.g. on reversed phase silica gel with gradients of 5-80% acetonitrile/0.1% TFA in H₂O/0.1% TFA. Fractions with pure compound, analyzed by MS, are pooled and solvent is evaporated.

For reagents with Hexa-histidine and APP tags the cleavage of the peptide reagent from the resin and the simultaneous cleavage of the acid-labile protection groups is performed by 2 to 3 treatments of the resin for up to 5 hours each under occasional agitation with about 3 bed volumes of a mixture from 0.75 g phenol, 0.25 ml 1,2-EDT, 0.5 ml thioanisol, 0.5 ml H₂O, 10 ml TFA (Kronina V. V., Wirth H. J., Heam M. T. W., J. Chromatography (1999) 852(1), 261-272), similar to Reagent K (King D. S., Fields C. G., Fields G. B., Int. J. Pept. Protein Res. (1990), 36, 255). After each cleavage the supernatant obtained is washed 3 times with DCM and collected in glass flasks. Solvent is evaporated, the residue obtained is flushed with argon, taken up in some MeOH and again solvent is evaporated. The combined residues obtained are extracted under argon 3 times with ethylether and solvent is evapoarted. The compounds obtained are dissolved in DMF and optionally purified by e.g. HPLC as described above.

1.2. Labeled peptide reagents for reaction with single internal or terminal cysteines of target peptides or target proteins are prepared by first synthesizing a backbone structure without reactive N-terminal group, selective deprotection and addition of the dye-label into the side chain followed by N-terminal deprotection and addition of a cysteine-reactive functional group e.g. maleimido-alkyl carboxylic acid, iodoacetyl- or vinylsulfone-group, followed by removal of remaining protection groups and cleavage from support.

The synthesis of the backbone of the peptide reagent resin on solid support with affinity tag, spacers and alternatively linkers is done with HOBt/DIC or TBTU/DIPEA activation as described in 1.1. The lysine for attachment of the dye is introduced as α-N-Fmoc-ε-N-4-methyltrityl-protected L-lysine with DIC/HOBt activation and the backbone is elongated to an N-terminal N-Fmoc-glycine.

The very acid labile 4-methyltrityl group at the lysine side chain is cleaved by repeated treatments with 5% TFA/DCM for a total of 30 minutes without cleaving the Rink linker. The resin obtained is washed 5 times with DCM and then DMF. A 0.15 M solution of 1.5 eq. of dye in the form of its carboxylic acid in anhydrous DMF is added to the resin, cooled to 4°, 1.5 eq. of solid PyBop are added and mixed, 8 eq. of DIPEA are added and the mixture obtained is agitated for 17 hours at 4°. Alternatively the resin is reacted with 1.5 eq. of the dye in the form of its carboxylic acid N-hydroxysuccinimidyl ester in anhydrous DMF for 16 hours at rt. The resin obtained is drained and extensively washed with DMF, MeOH and DCM. The N-terminal Fmoc-group is cleaved off as described, 4 eq. of 4-maleimido-butyric acid, 0.1 M in anhydrous DMF are added. 4 eq. of PyBop and 6 eq. of DIPEA are added and the resin obtained is agitated for 17 hours at 4°, washed with DMF, MeOH, DCM and dried. Alternatively the resin can be reacted with 4 eq. of iodoacetic acid, 4 eq. of PyBop, 6 eq. of DIPEA or with 10 eq. of divinylsulfone.

The acidic cleavage is performed according to the method described in 1.1. The product obtained is optionally further purified by e.g. HPLC as described.

1.3. Labeled peptide reagents with reactive thioester function for native binding with an N-terminal cysteine of a target peptide or target protein are prepared on an solid support with amino groups (e.g. polystyrene or polystyrene-polyethylenglycol, Tentagel) functionalized with sulfonamidobutyl-carboxylic acid (sulfamylbutyryl resin). 4 eq. of N-Fmoc-protected glycine is coupled twice to the sulfonamido group with 4 eq. of PyBop/8 eq. of DIPEA at −18° for 12 hours (see e.g. Backes B. J. et al., J. Am. Chem. Soc. 1996, 118, 3055-6 or Backes B. J. et al, J. Org. Chem. 1999, 64, 2322-30). After Fmoc-deprotection with 20% piperidine/DMF and washings with DMF the sulfonamido-glycine derivatised support is elongated from C- to N-terminal direction to generate the peptide reagent in the sequence Y, D, Y′, X, Y″ by coupling procedures as described in example 1.1. The affinity tag in E (e.g. Biotin, His₆, APP) is positioned at the N-terminal part of the reagent. The N-terminal amino acid is introduced as N-Boc-protected derivative. After selective cleavage of the Dde- or ivDde protection group at ε-amine of lysine with 2% hydrazine/DMF treatment for 3 times 5 minutes and DMF washes, the dye label is coupled to the ε-amino group of the side chain as described in example 1.1. The sulfonamide linker is N-alkylated by agitation of the resin with surplus (about 50 eq.) of 1 M trimethylsilyidiazomethane in hexane/THF 1:1 for 1-3 hours, followed by washings with THF and DMF. The resin is repetitively reacted for 9-12 hours each with surplus (5 eq.) of either a mixture of 0.8 M of 2-MESNA and 0.024 M of sodium thiophenolate in DMF or 0.8 M of MPE and 0.024 M of sodium thiophenolate in DMF (see e.g. Ingenito R. et al., J. Am. Chem. Soc, 1999, 121, 11369-74) to generate crude labeled peptide reagent with C-terminal MESNA- or MPE-thioester The solutions obtained and the solutions obtained from two DMF washings of the resins are collected. The alkylation and cleavages may be repeated to maximally remove the thioester product. Solvent is evaporated. The combined solutions of crude MESNA-thioester derivatives, which contain a surplus of 2-MESNA as salt, are additionally purified on a column with e.g. hydrophobic adsorption resin, e.g. Amberchrom CG-161c (TosoHaas). An aqueous solution of the crude MESNA-thioester is applied to the H₂O-pre-equilibrated resin, the resin obtained is washed with H₂O, the product obtained is eluted with acetonitrile and solvent is evaporated. Alternatively the surplus 2-MESNA can be separated by elution of the crude product via a size exclusion column with H₂O.

After cleavage of the remaining N-terminal Boc-protection group by treatment with TFA/DCM 1:1 for 1 hour and evaporation of solvent, crude products obtained are 3 times extracted with diethylether and solvent is evaporated. Compounds obtained are dissolved in DMF and purified, e.g. by preparative reversed phase chromatography using acetonitrile gradients in 0.1% TFA. Dried compounds obtained are analyzed by MS.

Alternatively thioesters can be generated at the N-terminus of labeled peptide reagents, synthesized as in 1.1. or 1.2. by elongation with dicarboxylic acids or bifunctional reagents which allow the attachment of terminal amino acids or aminoalkylcarboxylic acids at their amino group. The terminal carboxylic acids are converted with suitable methods into N-terminal carboxylic acid thioesters.

### EXAMPLE 2

General Scheme for Labeling of a Target Protein or Target Peptide and Purification of the Labeled Protein or Labeled Peptide:

a) The target protein or target is prepared e.g. in the IMPACT™-CN- or IMPACT™-TWIN System [New England Biolabs], which generates a native but thioester-activated C-terminus of the target protein or target peptide or alternatively a target protein or target peptide with an additional cysteine at the N-terminus.

b) Transesterification of the generated C-terminal thioester with the N-terminal cysteine of an appropriately selected compound of formula I (peptide reagent) occurs by nucleophilic attack of the sulfur of the Cys-SH group to the carbon in the thioester group. The unstable peptide reagent-protein thioester or peptide reagent-peptide thioester intermediate is drawn out of equilibrium by an intramolecular shift to a stable native amide bond, which (i) shifts the equilibrium towards the reaction product and allows a nearly quantitative reaction and (ii) directs the binding (ligation) towards exclusive reaction with the N-terminal cysteine of the peptide reagent.

Alternatively, the transesterification occurs between the N-terminal cysteine of the target protein or target peptide and the thioester terminus (C- or N-terminal) of the labeled peptide reagent followed by intramolecular shift to a stable amide bond in a nearly quantitative reaction as described above.

In yet another alternative, a target protein or target peptide with a cysteine at the N-terminus may be reacted with an aldehyde function of the peptide reagent.

In yet another alternative, a target protein or target peptide containing only one internal N-terminal cysteine may also be reacted with a labeled peptide reagent containing an N-terminal maleimido-, iodoacetyl- or vinylsulfone-functionality, forming a stable thioether bond between the Cys-SH group and the functional terminus by addition, respectively substitution, in a nearly quantitative reaction.

c) Unreacted peptide reagent is removed, e.g. by size exclusion chromatography or by dialysis.

d) Remaining unlabeled target protein or unlabeled target peptide is removed by affinity chromatography using the affinity tag E of the compound of formula I (peptide reagent). This can be based on a reversible affinity interaction such as His₆ with Ni²⁺, FLAG tag with anti-FLAG antibody or APP tag with anti-APP antibody or an “irreversible” interaction such as biotin with streptavidin or avidin, or a direct covalent bond to a solid support.

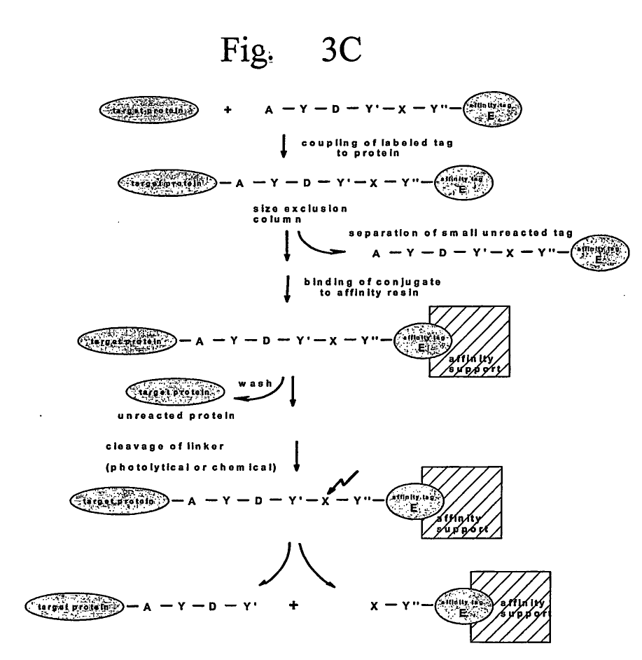

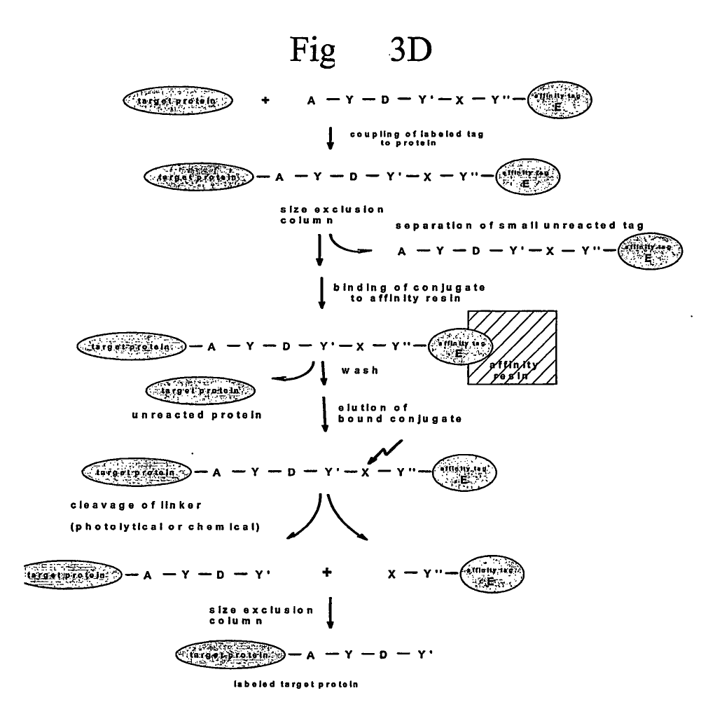

e) Labeled bound protein or peptide is either recovered from reversible affinity columns and the affinity tag is removed if desired by specific cleavage of the linker in solution, either by photophysical or by chemical means, or the labeled bound protein or peptide is specifically cleaved on the support at the internal linker, either by photophysical or by chemical means, and released form the support, optionally prepurification, e.g. size exclusion chromatography carried out before affinity purification (see e.g. FIGS. 3A to 3D).

f) Optionally hydrophilic spacing residues of variable length are present between e.g. the cleavable linker or the label containing residue and the affinity tag for e.g. optimization of positions and solubility properties.

### EXAMPLE 3

Labeling of the Protein HuR12 as a Target Protein

For site-specific labeling of the C-terminal thioester of HuR12 a chemically highly stable thioester is generated with 2-MESNA. Accordingly, LC/ESI MS reveals more than 98% of the protein being still present as C-terminal thioester after several purification steps (affinity chromatography, preparative RP-HPLC, lyophilization). In contrast, thioesters prepared with DTT are already hydrolysed to more than 60% after analogous treatment.

The procedure is typically performed as follows:

a) Coupling of HuR12-2-MESNA to e.g. a substance of example 8 at a peptide reagent-to-target protein ratio between 5:1 and 25:1 and pH 8.0 in presence or absence of 50 mM 2-MESNA for 15 hours at 4° and protected from light.

b) Purification

Unbound peptide reagent is removed by gel filtration (e.g. BioRad DG10 columns) and unlabeled target protein is removed via immobilisation of the affinity tagged protein to e.g. Streptavidin Sepharose FF [AP Biotech].

c) Cleavage of the Linker

Labeled target protein is recovered by on-column photocleavage of the linker residue for 60 minutes at 365 nm, 3 mW/cm² [UV STRATALINKER 1800] in a glass vial and under stirring.

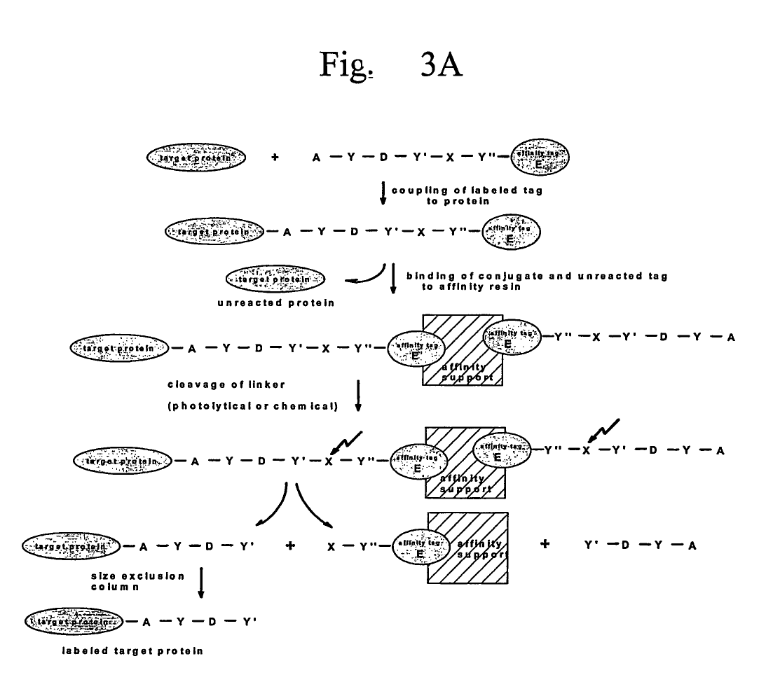

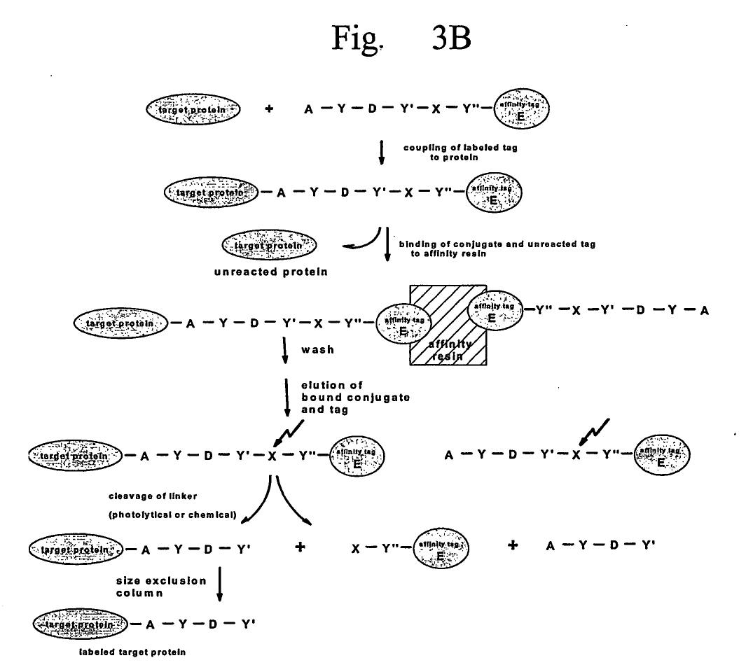

A 1:1 labeling stoichiometry and >99% purity for the labeled protein is confirmed by LC/ESI-MS (see FIG. 2) and RP-HPLC analysis, whereas CD-spectroscopy reveals that the secondary structure of the protein is not affected by the affinity tag as compared to the CD-spectrum of the unlabeled protein.

### EXAMPLE 4

N-Terminal Ligation

N-terminal ligation of the peptide Cys-Gly-Lys-Gly-His-His-His-His-His-His and of calcitonin Cys-Ser-Asn-Leu-Ser-Thr-Cys-Val-Leu-Gly-Lys-Leu-Ser-Gln-Glu-Leu-His-Lys-Leu-Gln-Thr-Tyr-Pro-Arg-Thr-Asn-Thr-Gly-Ser-Gly-Thr-Pro-NH2 with peptide reagents with C-terminal thioester, e.g. compounds of examples 26 to 32, is carried out as follows:

4 nmol of compounds from 1 mM stock solutions In DMF and 4 nmol of the peptide with N-terminal Cys are dissolved in 150 μl PBS pH 7.3. 20 nmol of TCEP are added and reacted for 12 to 24 hours at 40.

Alternatively the reaction can be performed as described above but including 1% v/v thiophenol and reacting for 9 hours at 4°. The solution obtained is extracted 3 times with 50 μl diethylether. Labeled peptide is obtained, which may be further purified via its affinity tag as described in examples 2 or 3.

### EXAMPLE 5

Site-Specific, Internal 1:1 Labeling of HuR12 at Cys₁₃.

A compound of example 33 is coupled to the single cysteine residue at position 13 in the sequence of a shortened variant of the protein HuR (ELV1, Celera identifier CRA I hCP40023.2) encompassing amino acids 1-189 only (HuR12). 367 nmol of the peptide reagent of example 33 are dissolved in 10 μl of anhydrous DMF under argon atmosphere and added to 200 μl of 126 μM HuR12 in PBS (molar reagent: protein ratio of 14.6:1). The reaction is allowed to proceed over night at 4° C., protected from light and under gentle mixing. The reaction mixture obtained is passed over a DG-10 gelfiltration column (BIO-RAD) equilibrated with PBS and a purified reaction mixture is obtained.

Streptavidin Sepharose (Amersham Pharmacia; capacity: 8.6 nmol Biotin-BSA/100 μl gel) is equilibrated with the same buffer and added to the purified reaction mixture obtained above. The added capacity is kept below 50% of the amount of labeled protein. The suspension obtained is incubated for 30 minutes at 4° on a horizontal shaker, or until the supernatant has turned colourless. The beads obtained are washed with at least 10 bed volumes coupling buffer and unspecifically bound proteins are removed.

Labeled proteins are cleaved off from the Streptavidin matrix attached via Biotin-tag as the affinity tag by on-column photocleavage. Beads obtained are suspended in the desired target buffer and transferred into sealable glass vials. Photocleavage is performed in a Stratalinker® 1800 UV Crosslinker (Stratagene) at 365 nm and 3 mW cm⁻², for 60 minutes under stirring and cooling. The supernatant obtained is transferred into a fresh tube and the beads obtained are rinsed once with the corresponding buffer. Internally labeled protein is obtained.

### EXAMPLE 6

C-Terminal 1:1 Labeling of BIR-3

The BIR-3 domain NFPNSTNLPRNPSMADYEARIFTFGTWIYSVNKEQLARAGFYALGEGDKVKCFHCGGGLTD WKPSEDPWEQHAKWYPGCKYLLEQKGQEYINNIHLTHSLEECLVRTTEK encompassing amino acids 249-358 from the 497 amino acid sequence of human XIAP (total sequence SWISS PROT Database entry P98170; see e.g. Z. Liu et al., Nature (2000), 408, 1004-1008) is cloned into the NdeI/SapI restriction sites of the vector pTYB1 (IMPACT™-system, New England Biolabs) and purified as described for the protein SAP. Intein-mediated on-column cleavage is induced by addition of 2-MESNA (50 mM final concentration; Na-salt). The protein used for labeling experiments Is stored at −80° C. in a buffer of 20 mM Hepes pH 8.0, 500 mM NaCl, 1 mM EDTA, 50 mM 2-MESNA and at a concentration of 14.2 μM. 108 nmol of a compound of example 14 are dissolved in 20 ml of anhydrous DMF under argon atmosphere and added to 3.4 ml of 14.9 μM BIR-3 in a buffer of 20 mM Hepes pH 8.0, 500 mM NaCl, 1 mM EDTA, 50 mM 2-MESNA. (molar reagent: protein ratio is 2.8:1). The reaction is allowed to proceed for 48 hours at 40 on a horizontal shaker, protected from light. The reaction mixture obtained is passed over a DG-10 gel filtration column (BIO-RAD) previously equilibrated with a buffer of 50 mM NaH₂PO₄ pH 8.0, 300 mM NaCl and labeled protein is obtained.

Ni-NTA agarose (Qiagen) is equilibrated with the binding buffer. An amount of resin providing a capacity of at least twice the amount of labeled protein is added to the labeled protein obtained. The suspension obtained is incubated at 40 on a horizontal shaker for 4 hours, or until the supernatant has turned colourless. The beads obtained are washed with at least 10 bed volumes of coupling buffer containing 15-20 mM imidazole. Labeled protein is obtained by eluting either with 250 mM imidazole in coupling buffer or a buffer of pH <6.0. The eluate obtained containing the labeled protein is transferred into sealable glass vials and incubated for 60 minutes in a Stratalinker® 1800 UV Crosslinker (Stratagene) at 365 nm and 3 mW cm⁻², under stirring and cooling. The mixture obtained is passed over a DG-10 gel filtration column equilibrated with the final storage buffer and labeled protein without affinity tag is obtained.

### EXAMPLE 7

Preparation of C-Terminally 1:1 Labeled, Soluble HuR_{fl}

The solubility of the highly hydrophobic protein HuR can be improved by attachment of a hydrophilic peptide tag to its C-terminus. A tag offering both, His₆ or FLAG, and a hydrophilic ADO spacer, allows to keep the protein in solution. A peptide reagent comprising a His₆ tag, two ADO spacers and a Cy5 fluorophore but no photo cleavable linker (e.g. a compound of example 25) is fused to the C-terminus of HuR. Labeled protein containing the hydrophilic tag is obtained.

236 nmol of a compound of example 25 are dissolved in 100 μl of anhydrous DMF under argon atmosphere and added to 12 ml of 420 nM thioester-activated full length HuR (ELV1, Celera identifier CRA I hCP40023.2, amino acids 1-326, HuRn) in a buffer of 20 mM Tris/Cl pH 8.0, 800 mM NaCl, 1 mM EDTA, 50 mM 2-MESNA, 0.2% Pluronic F-127 (Molecular Probes; molar reagent: protein ratio is 46.8:1). The reaction is allowed to proceed for 48 hours at 4° on a horizontal shaker, protected from light. The reaction mixture obtained is transferred into a Slide-A-Lyzer dialysis cassette (PIERCE) and dialysed against PBS at 4° C. (protected from light), unreacted peptide is removed and the buffer conditions are adapted to His₆-Ni-NTA agarose affinity purification.

Ni-NTA agarose (Qiagen) is equilibrated with the binding buffer. An amount of resin providing a capacity of at least twice the amount of labeled protein is added to the sample. The suspension obtained is incubated at 4° on a horizontal shaker for 4 hours, or until the supernatant has turned colourless. The beads obtained are washed with at least 10 bed volumes of coupling buffer containing 15-20 mM imidazole and labeled protein containing the hydrophilic affinity tag is obtained.

Analogously to the method as described in Example 1, but using appropriate starting materials, compounds of Examples 8 to 33 are prepared. In the corresponding examples 8 to 33 characterisation data (mass spectrometry data and from UV/VIS=ultraviolet and visible spectrum) of the compounds defined by formulae is set out.

ESI-MS spectra for reaction analysis and identification of HPLC fractions are recorded on a Finnigan Thermoquest Navigator MS system with Finnigan Aqa source, connected to a HP-1100 HPLC system. 20 μl aliquots from autosampler vials with 40-50 μl sample are introduced in MeOH and ionized in the positive mode at 250° and cone voltages of 25 and 50V. Detection range is between 200 to 1500 mass units, larger ions are detected by their multiple charged Ions.

The MALDI-TOF analysis is performed on an Applied Biosystems MALDI TOF Voyager STR. Typical conditions for analysis are: positive mode, accelerated voltage 25000V, 150 laser shots/sample, Laser intensity: 3035, Laser Rep. Rate: 35.5 Hz, acquisition range 500-8000 Da. As calibration matrix sinapinic acid saturated in AcCN/o.1% TFA 70:30 v/v is used.

UV-visible spectra are recorded in aqueous buffer 50 mM NaH₂PO₄, 100 mM NaCl, pH 7.0 on an Agilent 8453 spectrophotometer.

### EXAMPLE 8

A compound of formula

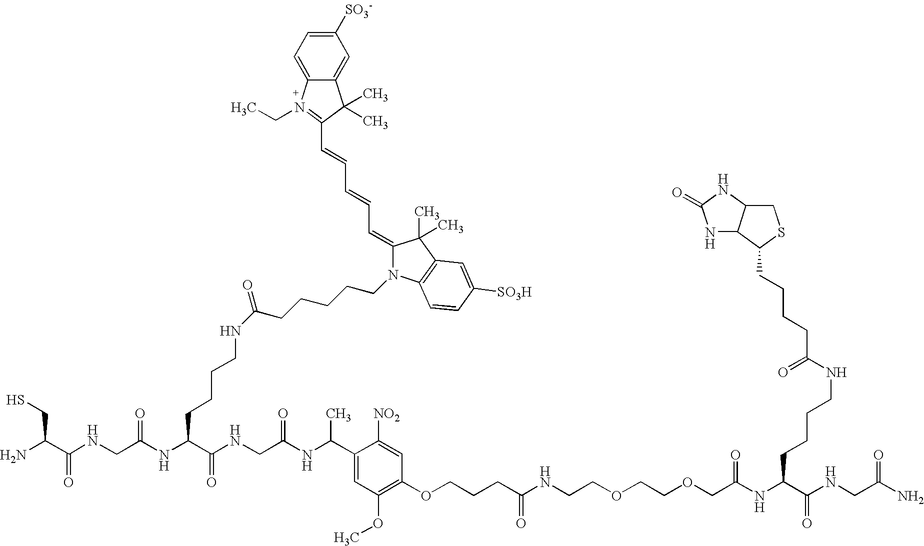

mw: 1839.24

ESI-MS: [M2H]²⁺919.4. MALDI-TOF MS: [MTFA]²⁻974.2 [MH]⁺1838.8

UV/VIS λₘₐₓ pH 7: 649 nm, sh 605 nm

### EXAMPLE 9

A compound of formula

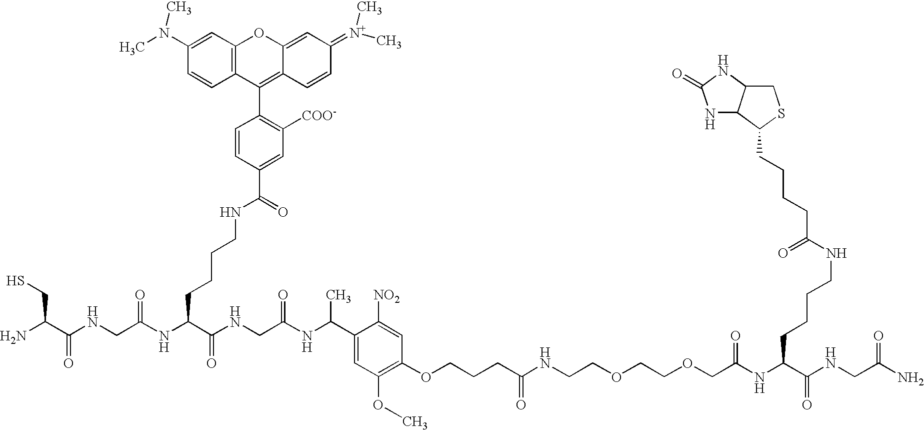

mw: 1612.88. ESI-MS: [M2H]²⁺806.1. MALDI-TOF MS: [MH]+1611.8. UV/VIS λₘₐₓ pH 7: 551 nm.

### EXAMPLE 10

A compound of formula

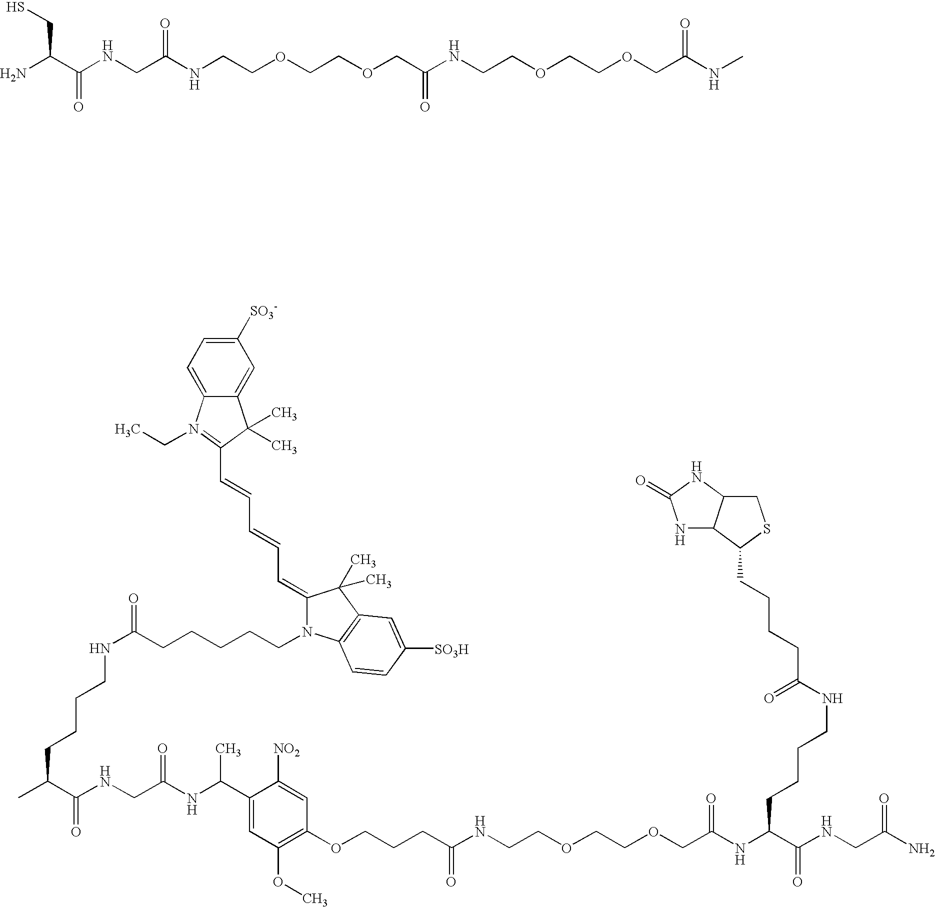

mw: 2129.56. ESI-MS: [M2H]²⁺1064.1. MALDI-TOF MS: [MH]⁺2128.5. UV/VIS λₘₐₓ pH 7: 649 nm, sh 605 nm.

### EXAMPLE 11

A compound of formula

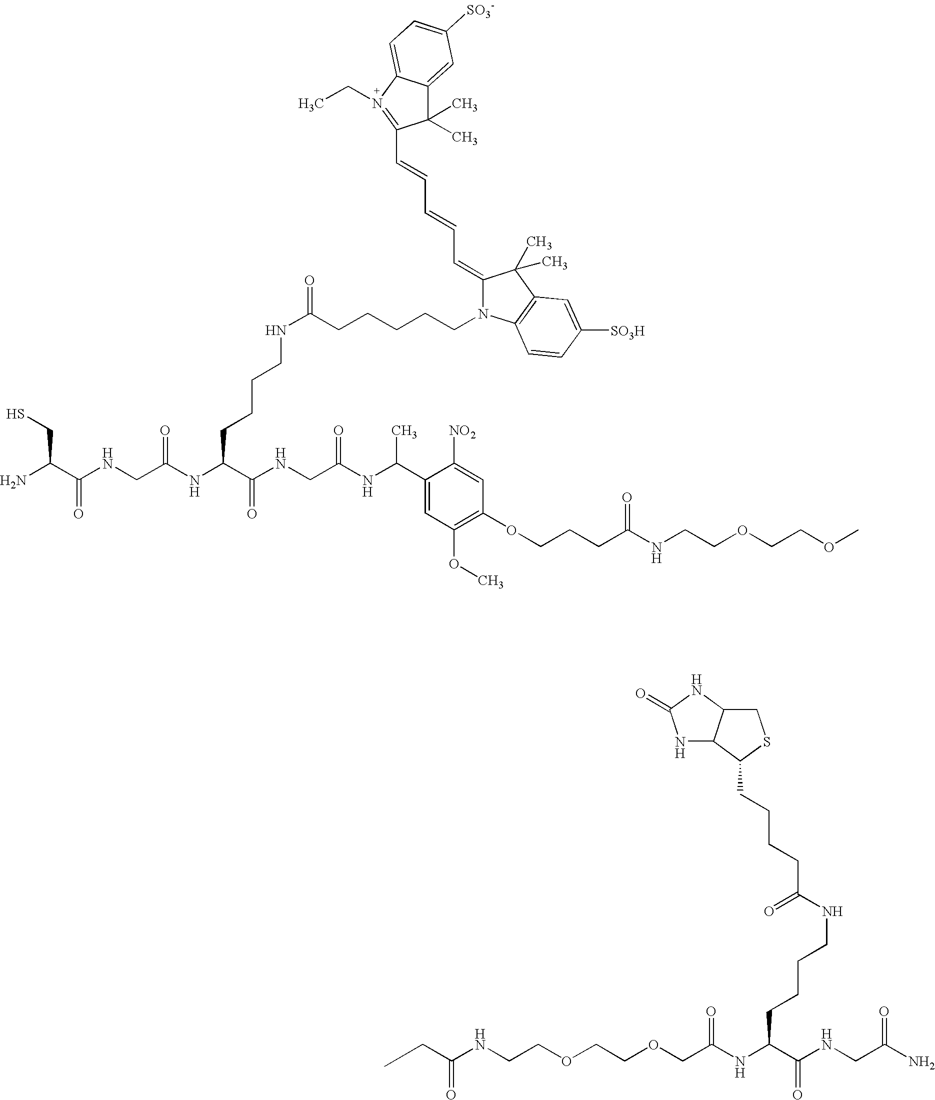

mw: 1984, 4. ESI-MS: [M2H]²⁺991.8, [MTFA]²⁻ 1046.6. MALDI-TOF MS: [MH]⁺ 1983.4. UV/VIS λₘₐₓ pH 7: 649 nm, sh 605 nm

### EXAMPLE 12

A compound of formula

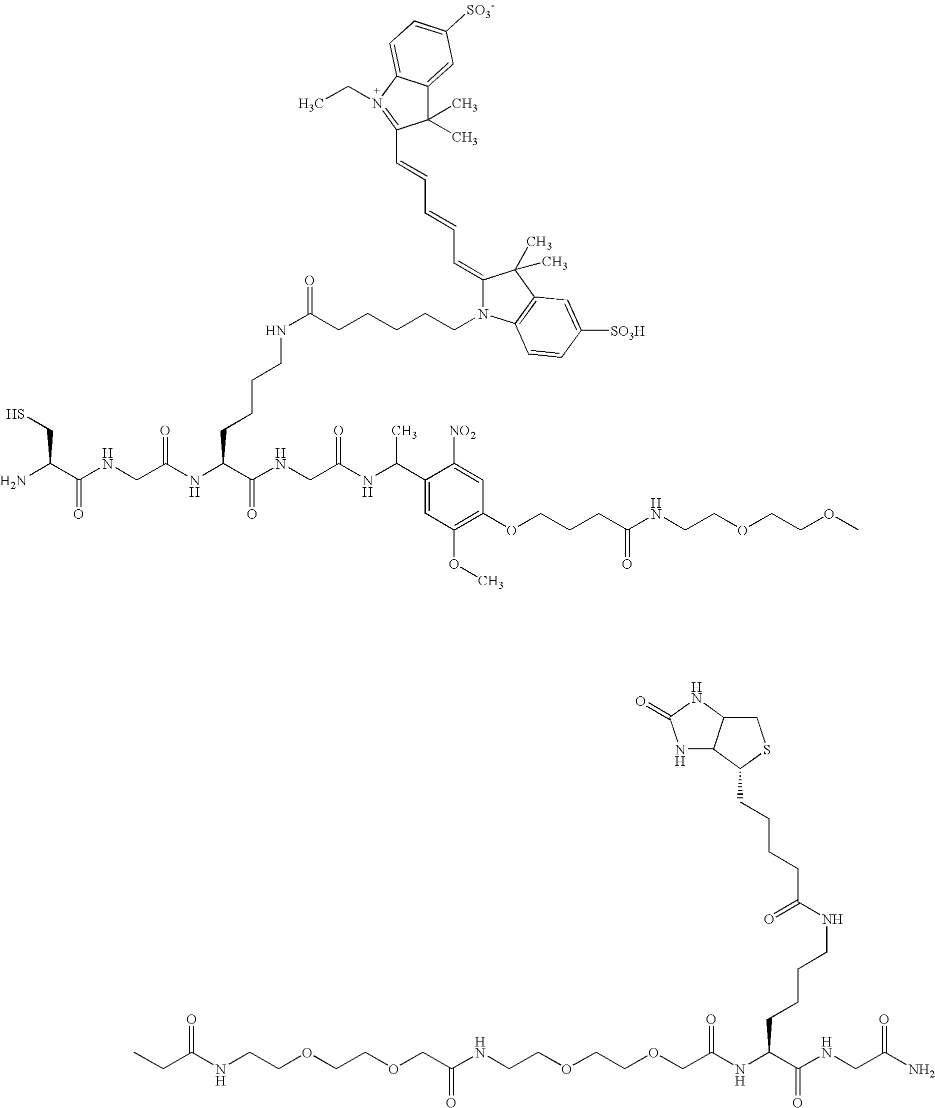

mw: 2129.56. ESI-MS: [M2H]²⁺ 1064.5, [MTFA]²⁻ 1118.9. MALDI-TOF MS: [MH]⁺ 2128.5 UV/VIS λₘₐₓ pH 7: 649 nm, sh 605 nm.

### EXAMPLE 13

### A compound of formula

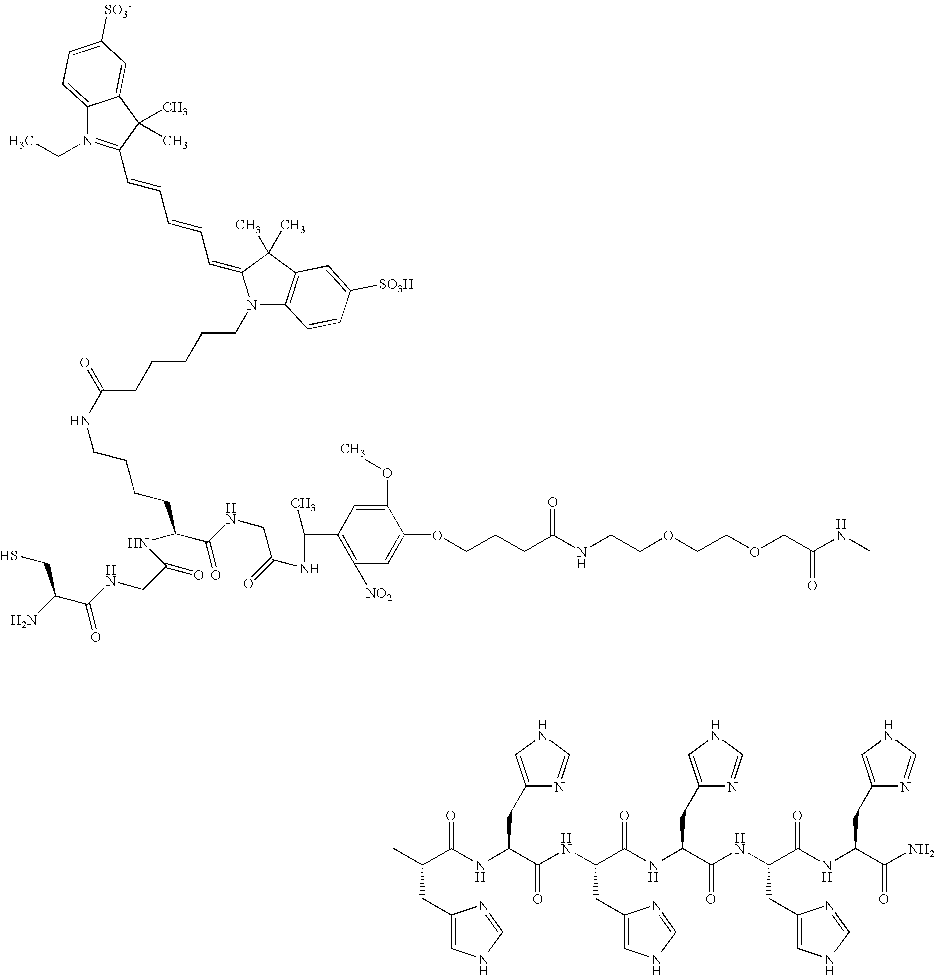

mw: 2250.57. ESI-MS: [M3H]³⁺ 750.1, [M4H]⁴⁺ 562.9. MALDI-TOF MS: [MH]⁺ 2249.9. UV/VIS λₘₐₓ pH 7: 649 nm, sh 605 nm.

### EXAMPLE 14

A compound of formula

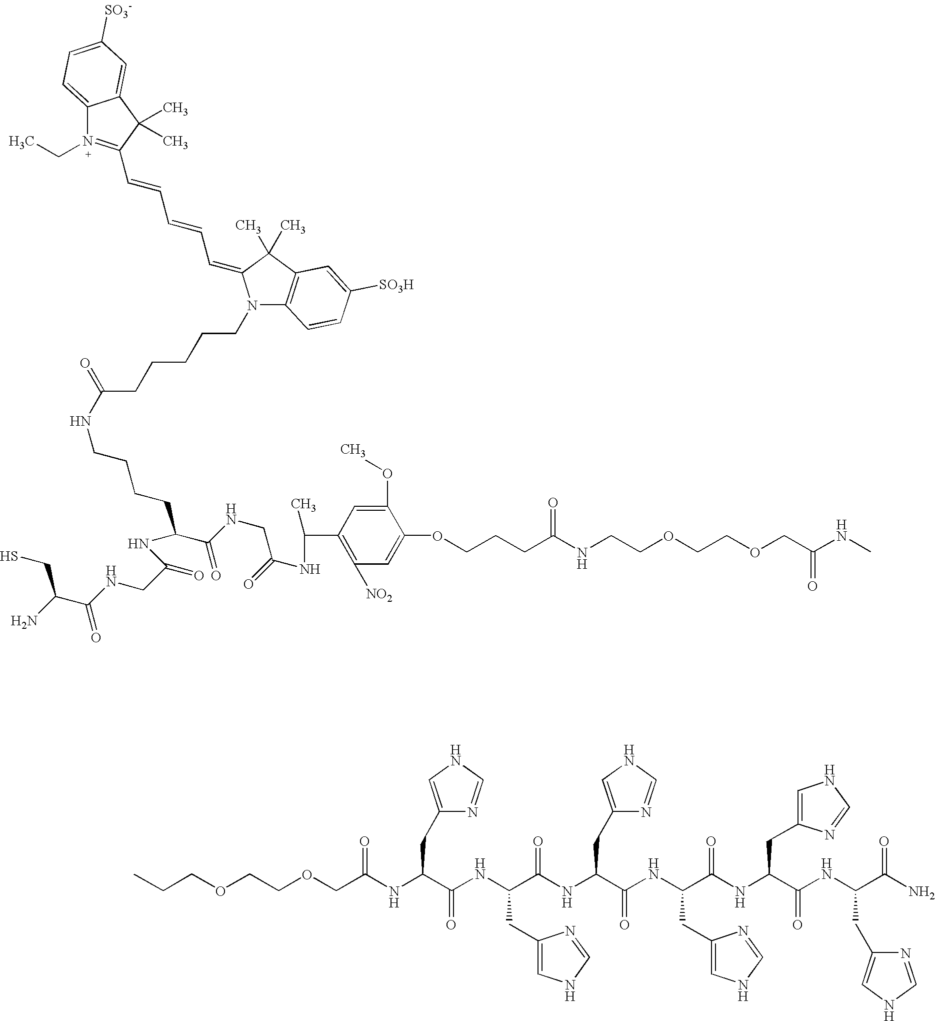

mw: 2395.72. ESI-MS: [M3H]³⁺ 798.8, [MTFA]²⁻ 1052.8. MALDI-TOF MS: [MH]⁺ 2395. UV/VIS λₘₐₓ pH 7: 649 nm, sh 605 nm

### EXAMPLE 15

A compound of formula

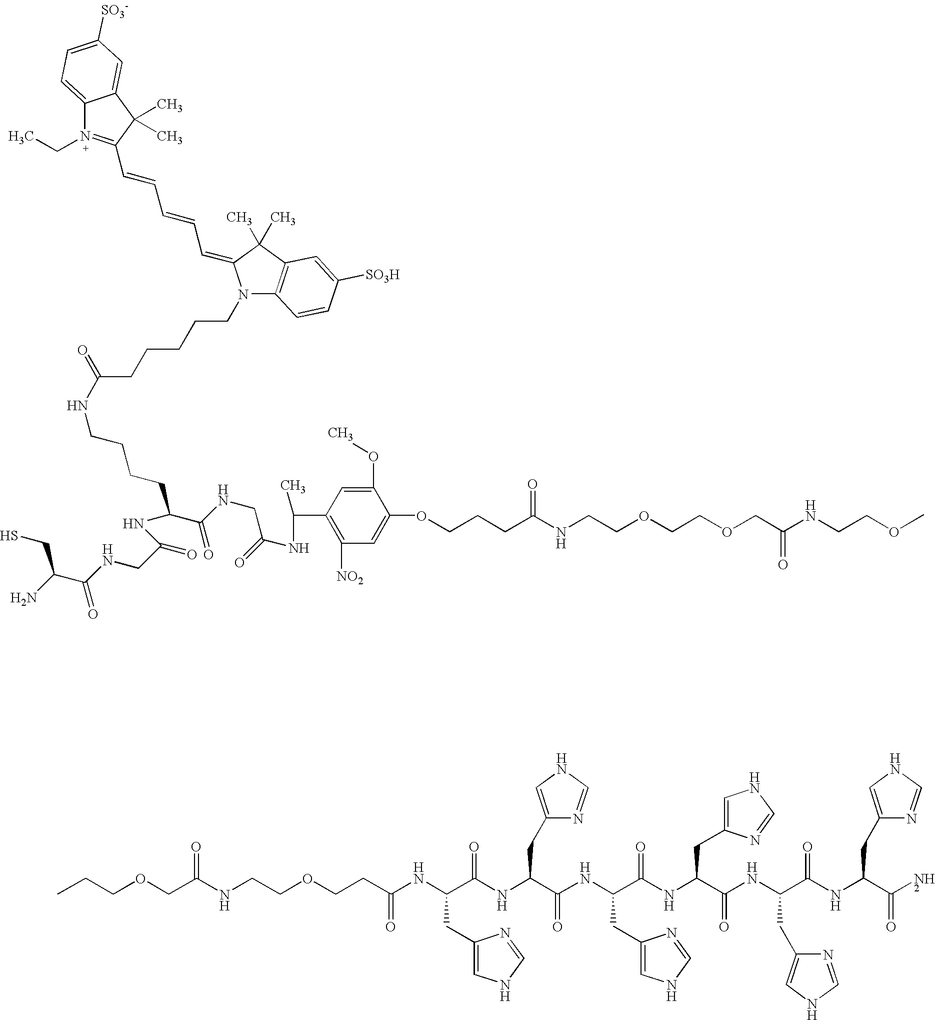

mw: 2540.88. ESI-MS: [M3H]³⁺ 846.8, [M4H]⁴⁺ 635.2. MALDI-TOF MS: [MH]⁺ 2540.0. UV/VIS λₘₐₓ pH 7: 650 nm, sh 606 nm

### EXAMPLE 16

A compound of formula

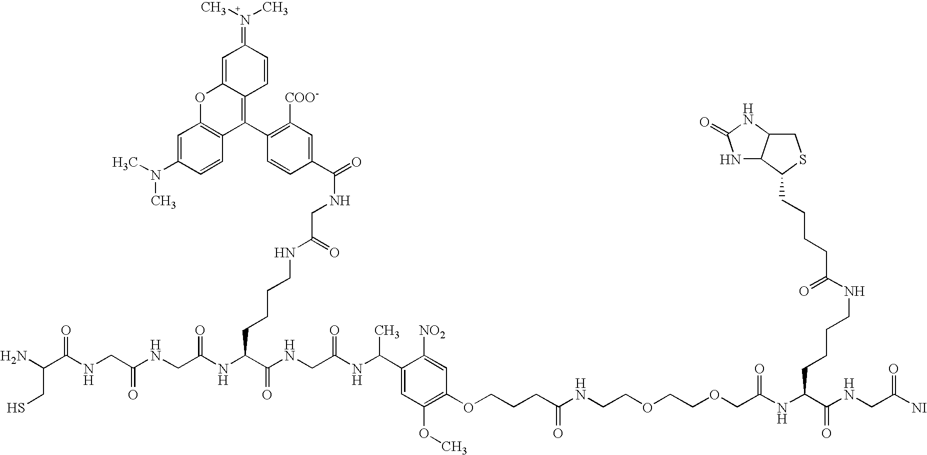

mw: 1726.98. ESI-MS: [MNa]²⁺ 873.7, [M2H]²⁺ 863.0. MALDI-TOF MS: [MH]⁺ 1725.8.

UV/VIS λₘₐₓ pH 7: 557 nm, sh 522 nm.

### EXAMPLE 17

A compound of formula

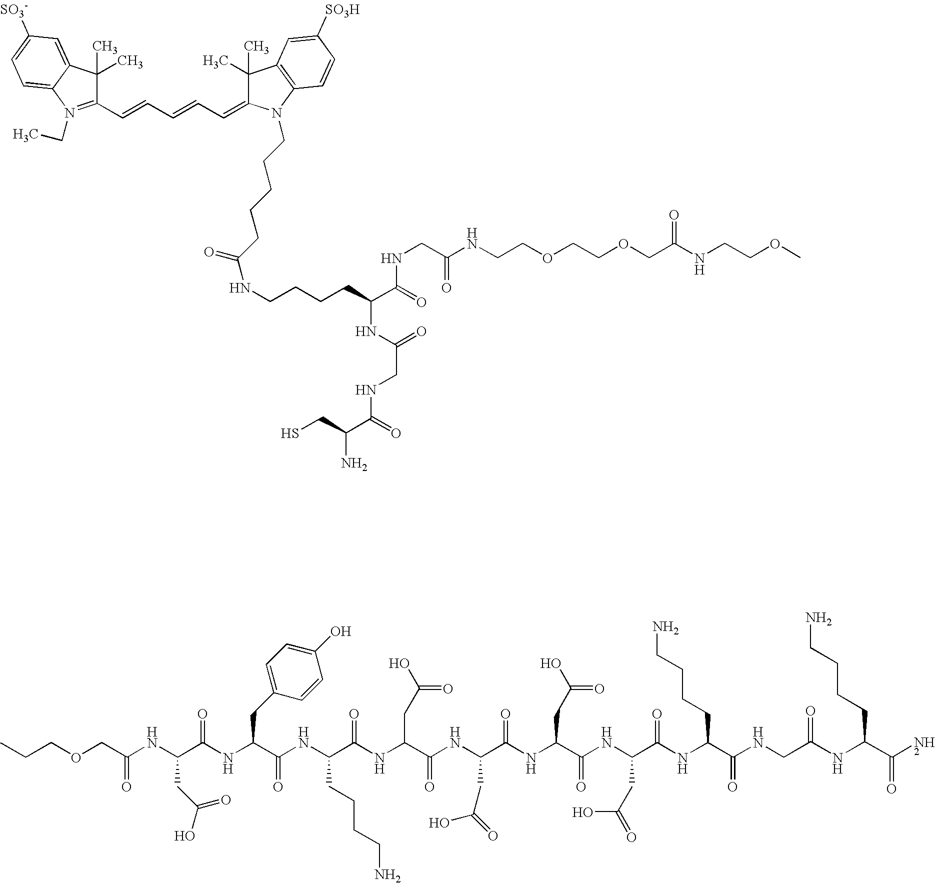

mw: 2472.79. ESI-MS: [M3H]³⁺ 824.2 [MNa]³⁺ 832.0. MALDI-TOF MS: [MH]⁺ 2472. UV/VIS λₘₐₓ pH 7: 648 nm, sh 605 nm

### EXAMPLE 18

A compound of formula

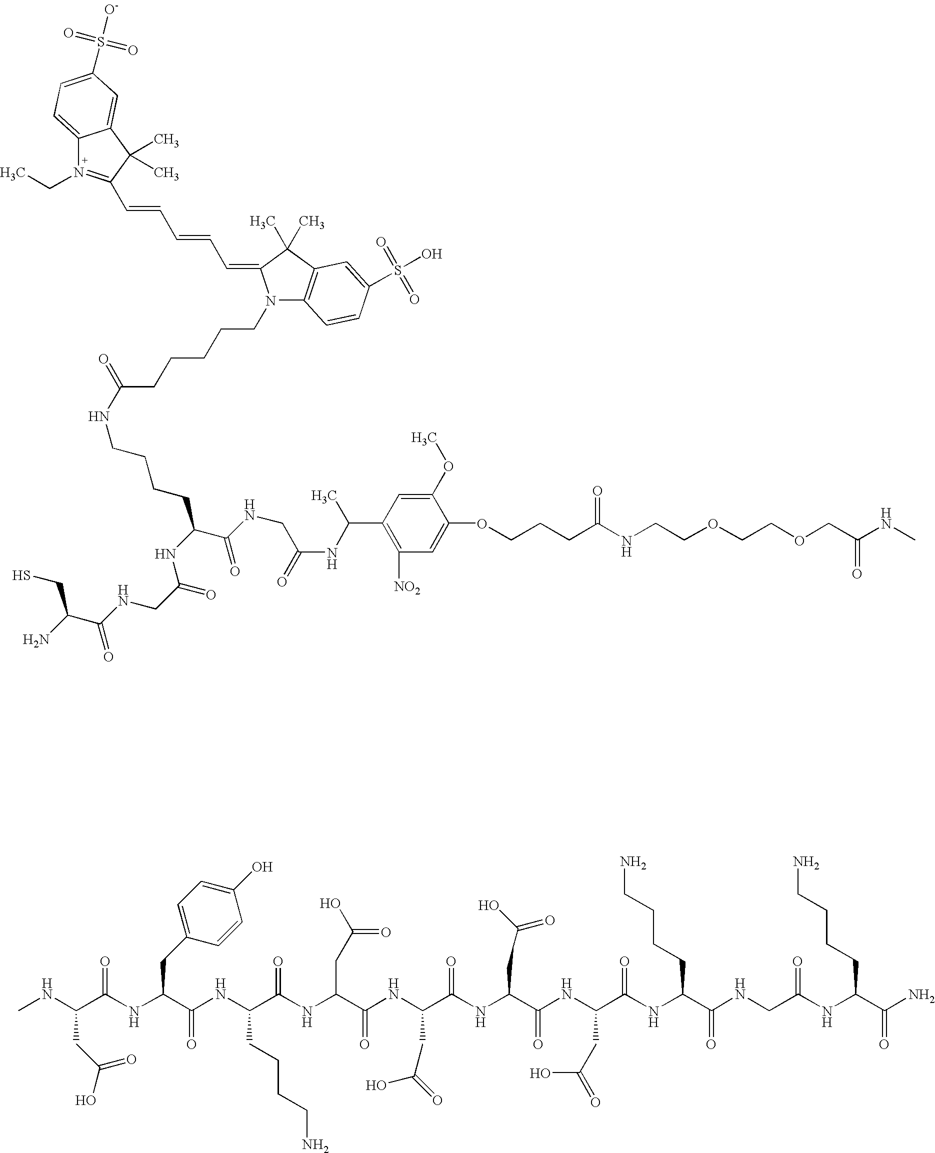

mw: 2607.91. ESI-MS: [M3H]³⁺ 869. MALDI-TOF MS: [MH]⁺ 2607. UV/VIS λₘₐₓ pH 7: 648 nm, sh 605 nm.

### EXAMPLE 19

A compound of formula

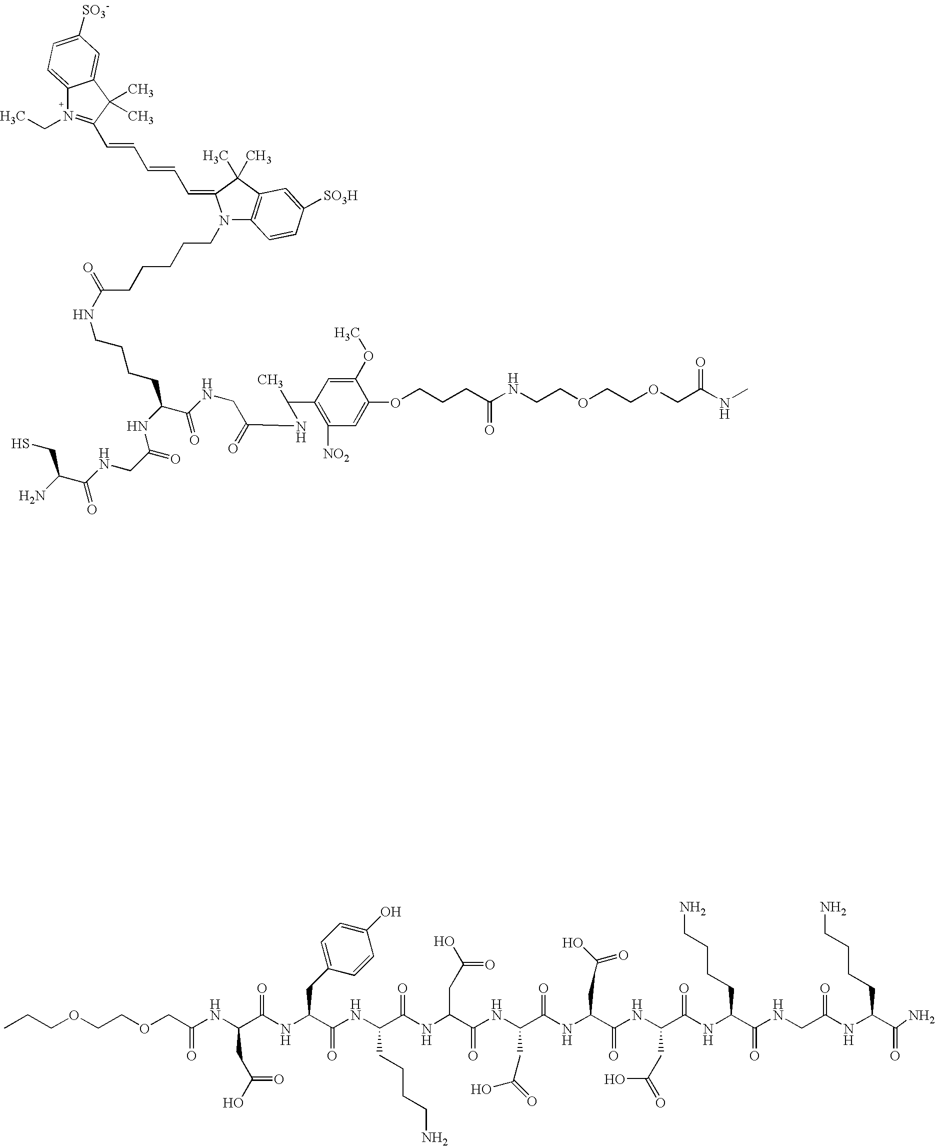

mw: 2753.07. ESI-MS: n.d. MALDI-TOF MS: [MH]⁺ 2752.2. UV/VIS λₘₐₓ pH 7: 649 nm, sh 605 nm.

### EXAMPLE 20

A compound of formula

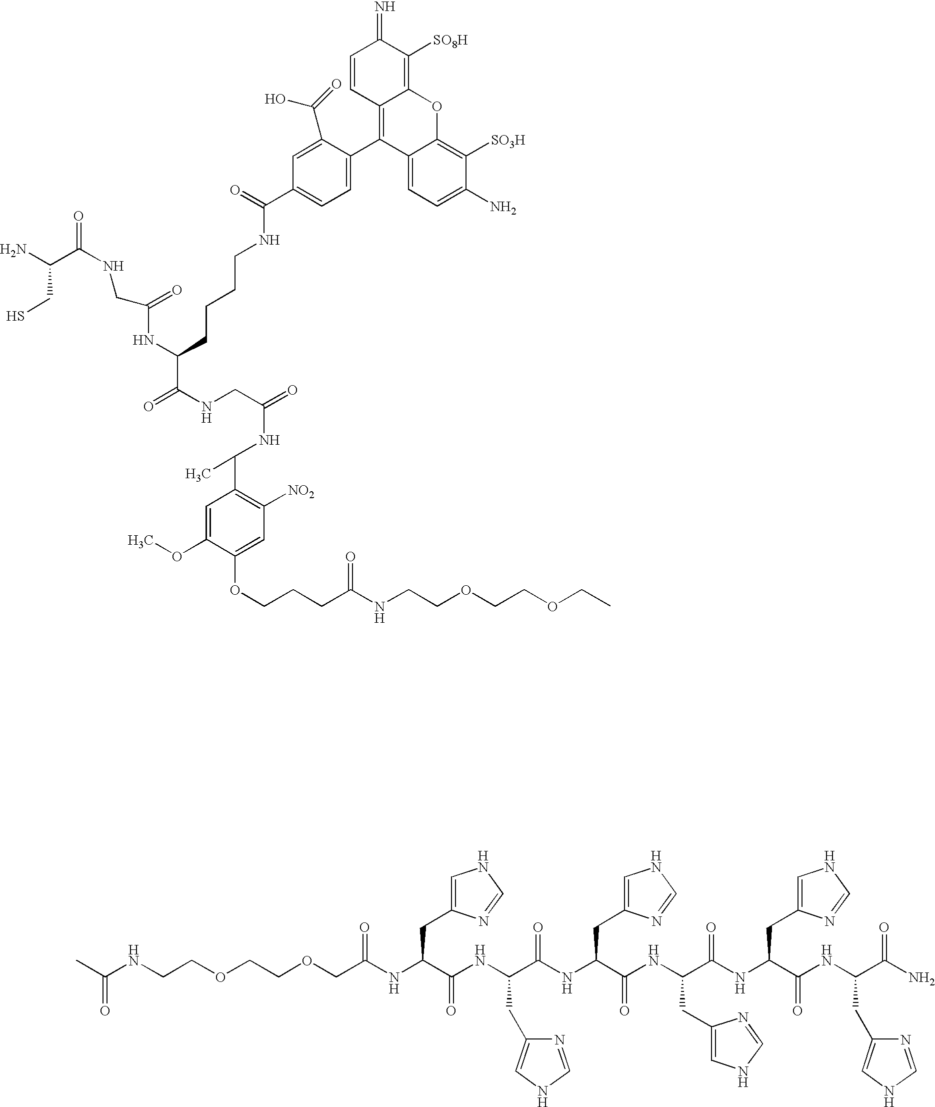

mw: 2272.37. ESI-MS: [M2H]²⁺ 1136.6, [M3H]³⁺ 757.8. MALDI-TOF MS: [MH]⁺ 2272.1. UV/VIS λₘₐₓ pH 7: 495 nm

### EXAMPLE 21

A compound of formula

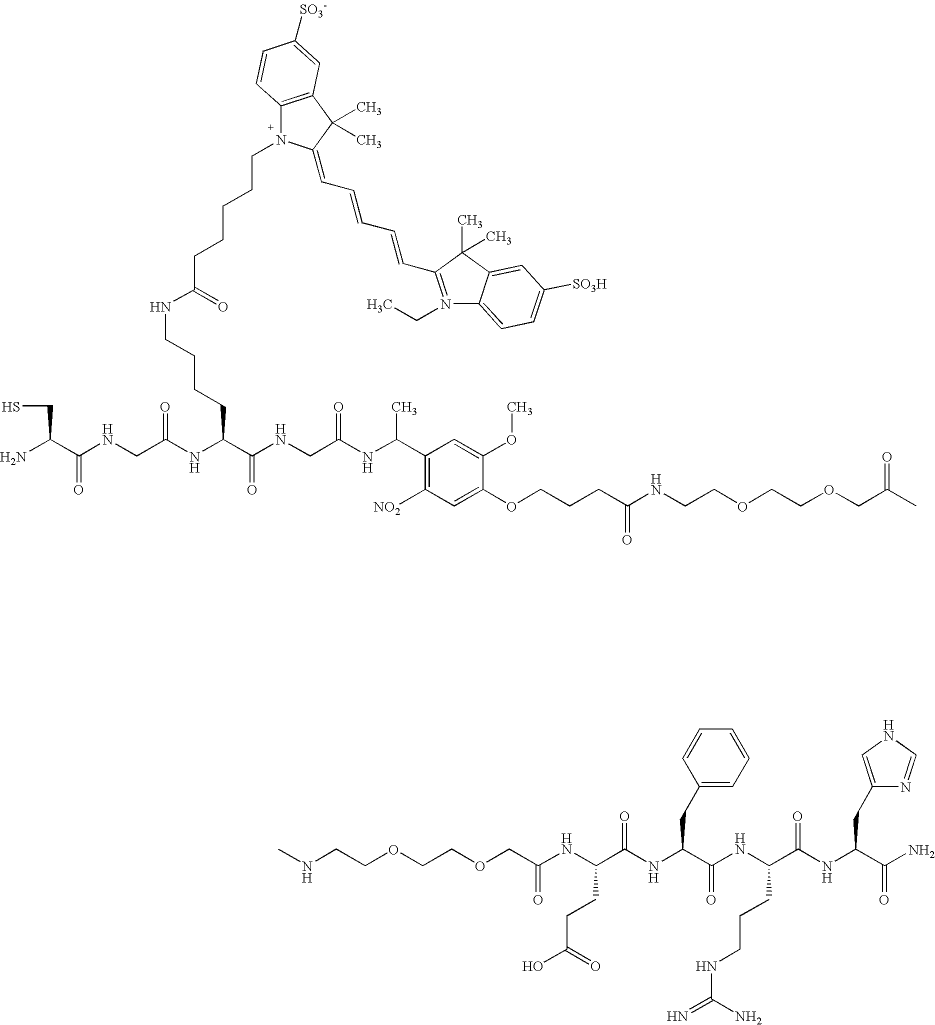

mw: 2142.5. ESI-MS: [M2H]²⁺ 1071.3. MALDI-TOF MS: [MH]⁺ 2141.7. UV/VIS λₘₐₓ pH 7: 649 nm, sh 605 nm

### EXAMPLE 22

A compound of formula

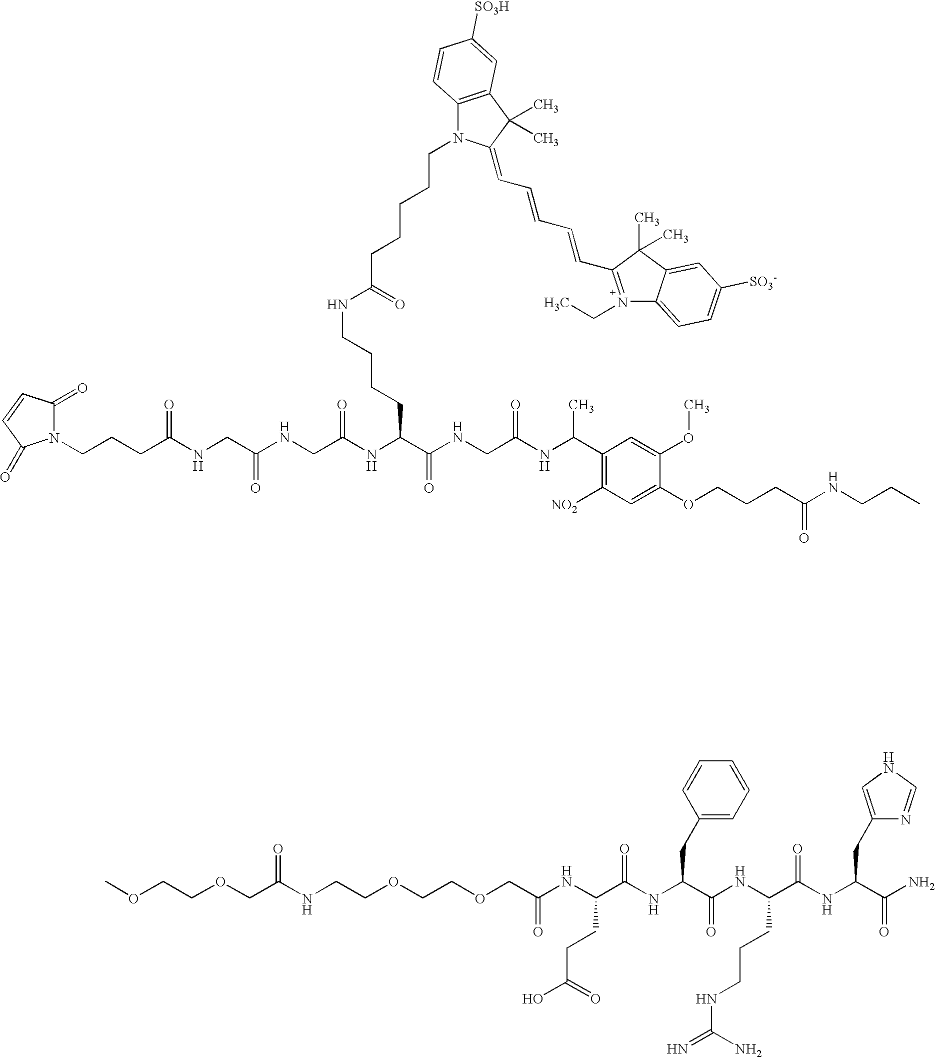

mw: 2261.56. ESI-MS: [M2H]²⁺1130.4, [M2HK]³⁺ 766.9. MALDI-TOF MS: [MH]⁺ 2262.9. UV/VIS λₘₐₓ pH 7: 649 nm, sh 607 nm

### EXAMPLE 23

A compound of formula

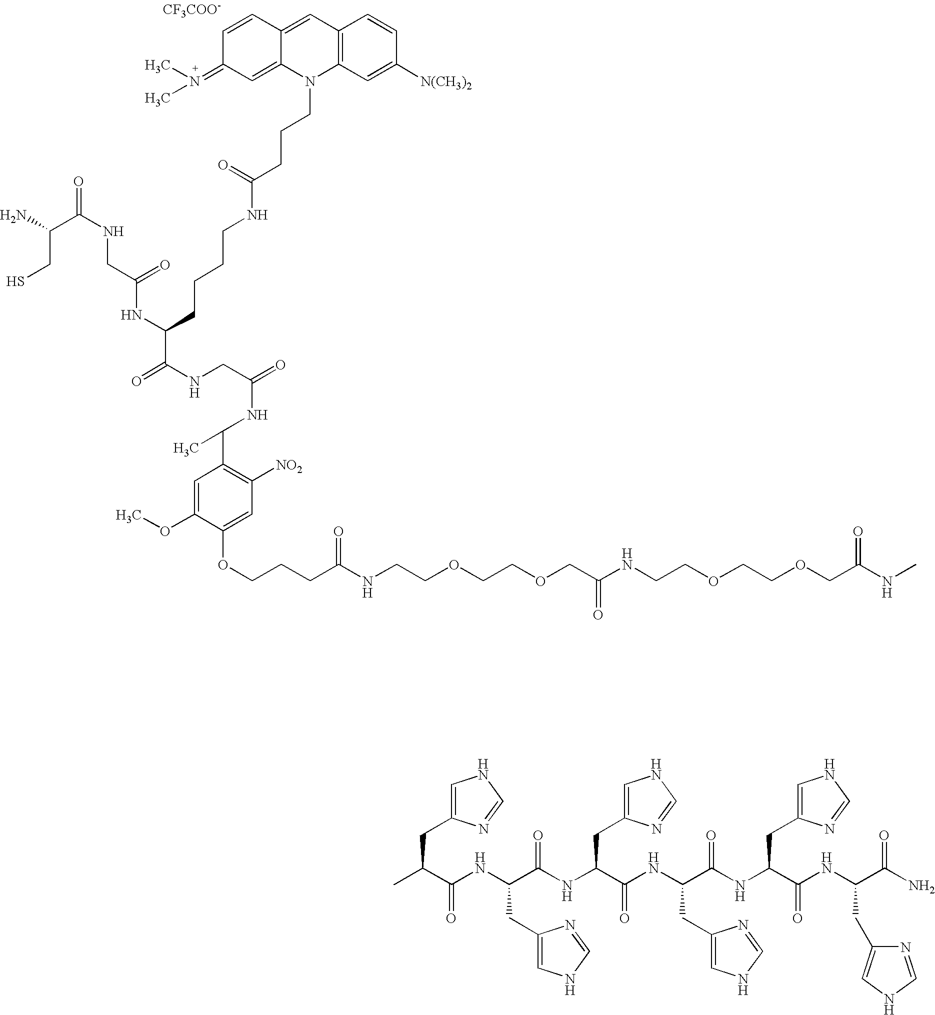

mw: 2090.35. ESI-MS: [M3H]³⁺ 697.2, [M4H]⁴+523.1, [M5H]⁵⁺ 418.8. MALDI-TOF MS: [MH]⁺2089.4. UV/VIS λₘₐₓ pH 7: 503 nm

### EXAMPLE 24

A compound of formula

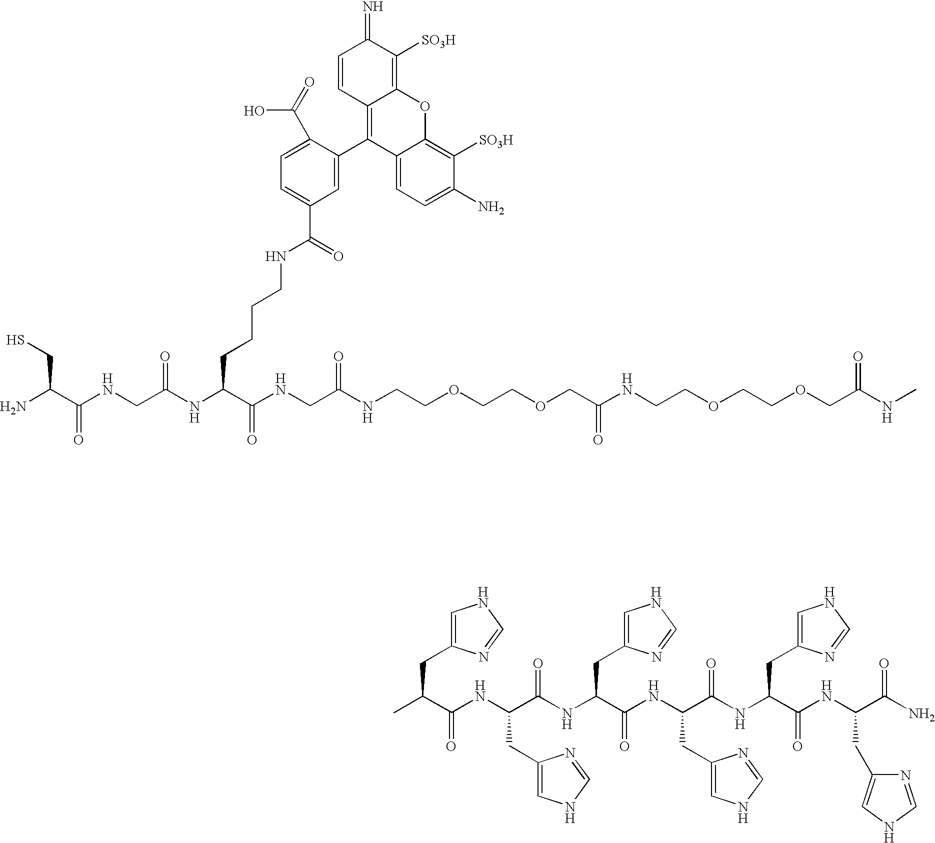

mw: 1992.09. ESI-MS: [M4H]⁴⁺ 498.7, [M3H]³⁺ 664.7. MALDI-TOF MS: [MH]⁺ 1992.39. UV/VIS λₘₐₓ pH 7: 494 nm.

### EXAMPLE 25

A compound of formula

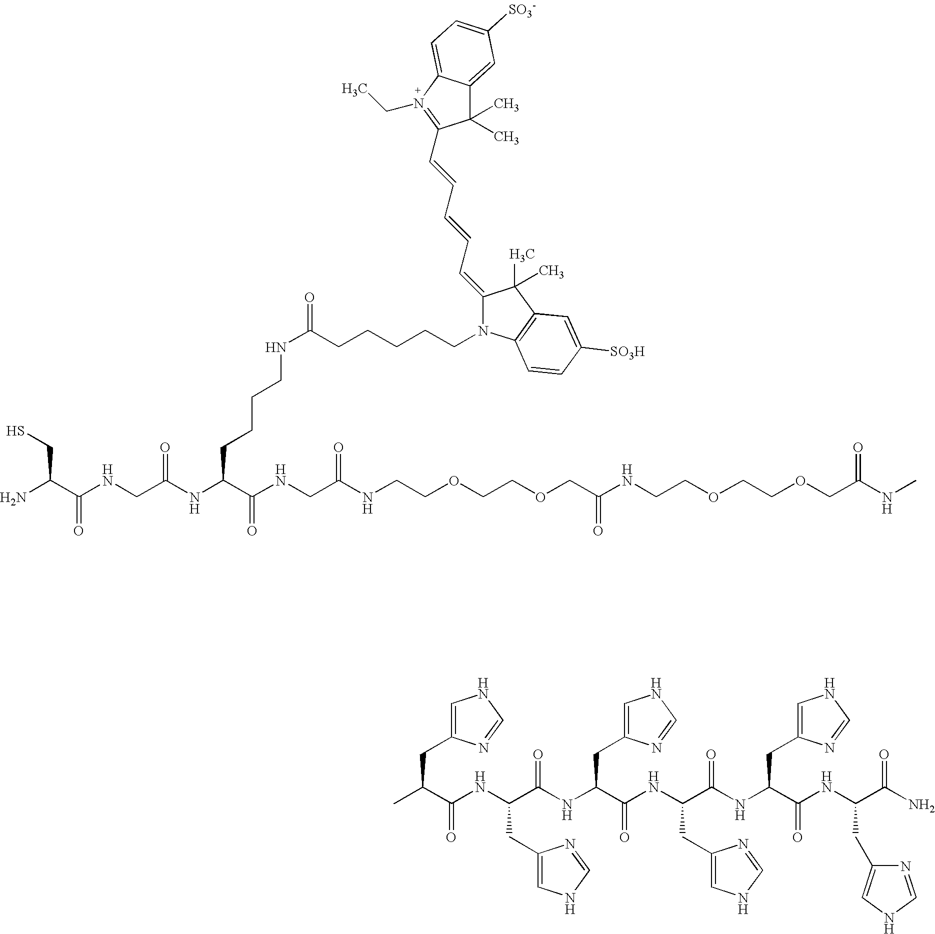

mw: 2115.44. ESI-MS: [M3H]³⁺ 705.7, [M4H]⁴⁺ 529.5. MALDI-TOF MS: [MH]⁺ 2114.6. UV/VIS λₘₐₓ pH 7: 649 nm, sh 605 nm.

### EXAMPLE 26

A compound of formula

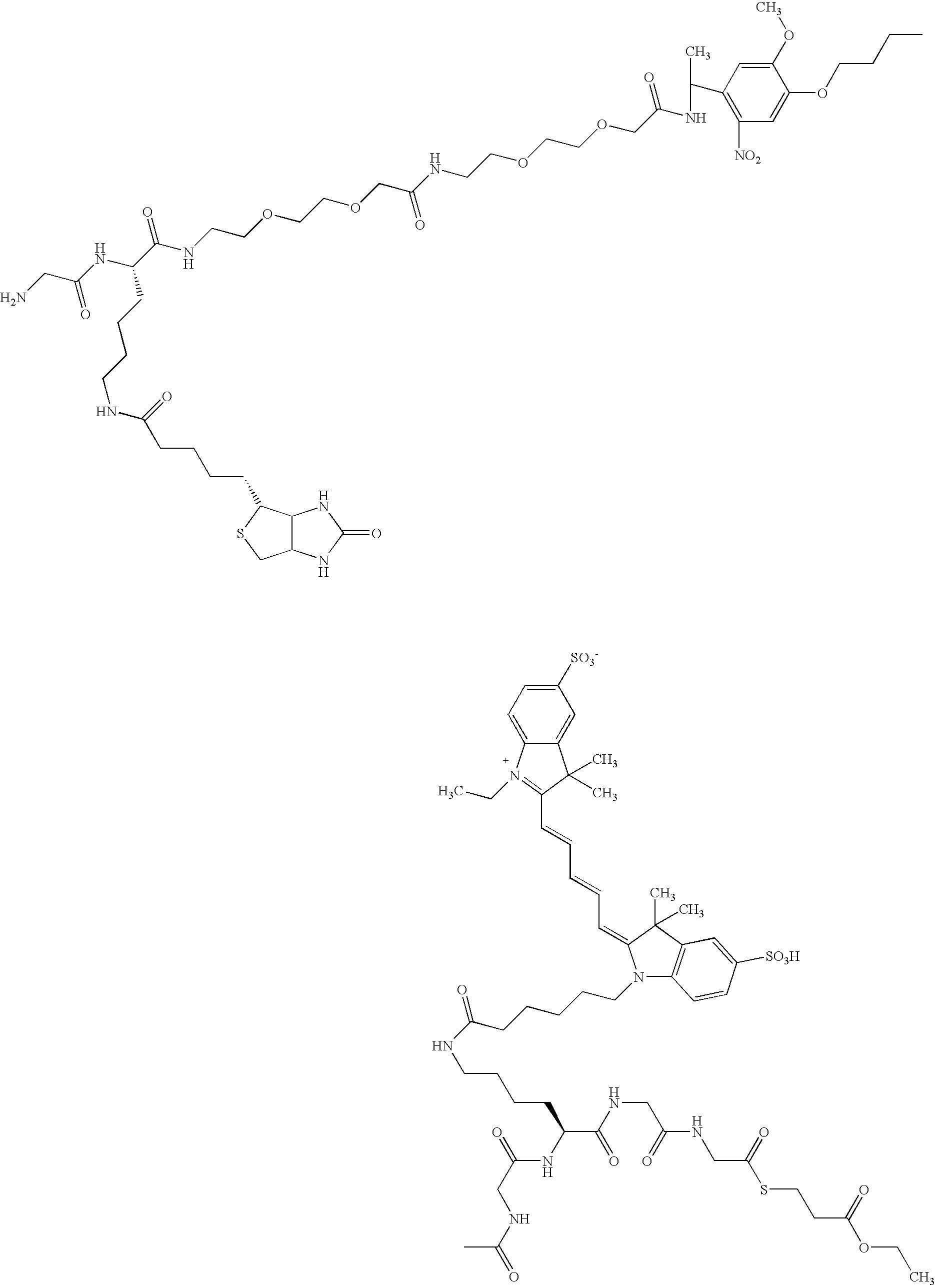

mw: 2055.48. ESI-MS: [M2H2K]⁴⁺ 533.3, [MH2K]³⁺ 710.6, [M2H]²⁺ 1027.9. MALDI-TOF MS: MH⁺ 2054.7, [MNa]+2076.7. UV/VIS λₘₐₓ pH 7: 649 nm, sh 605 nm

### EXAMPLE 27

A compound of formula

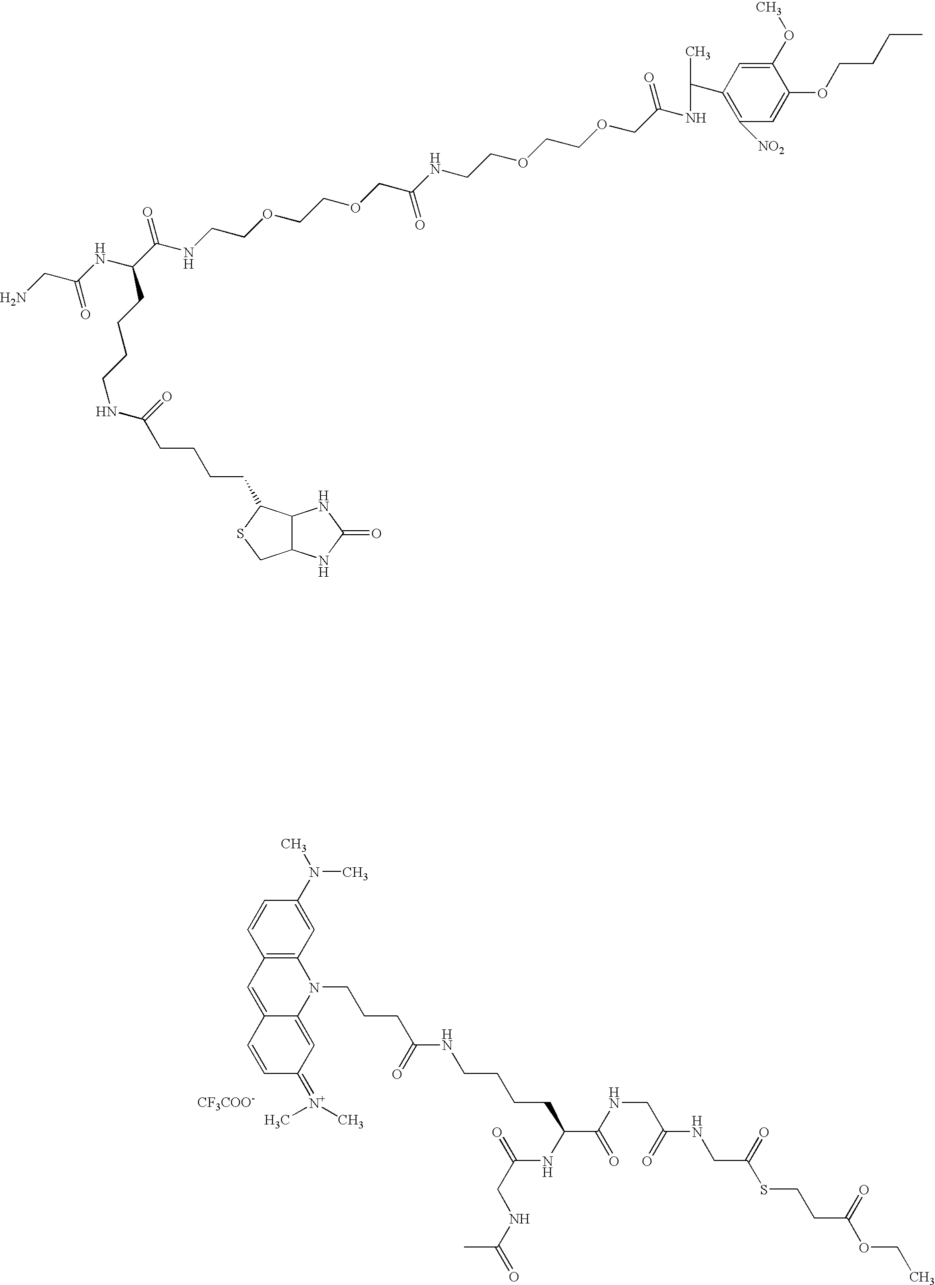

mw: 1750.1. ESI-MS: n.d, MALDI-TOF MS: [MH]⁺ 1748.9. UV/VIS λₘₐₓ pH 7: 503 nm

### EXAMPLE 28

A compound of formula

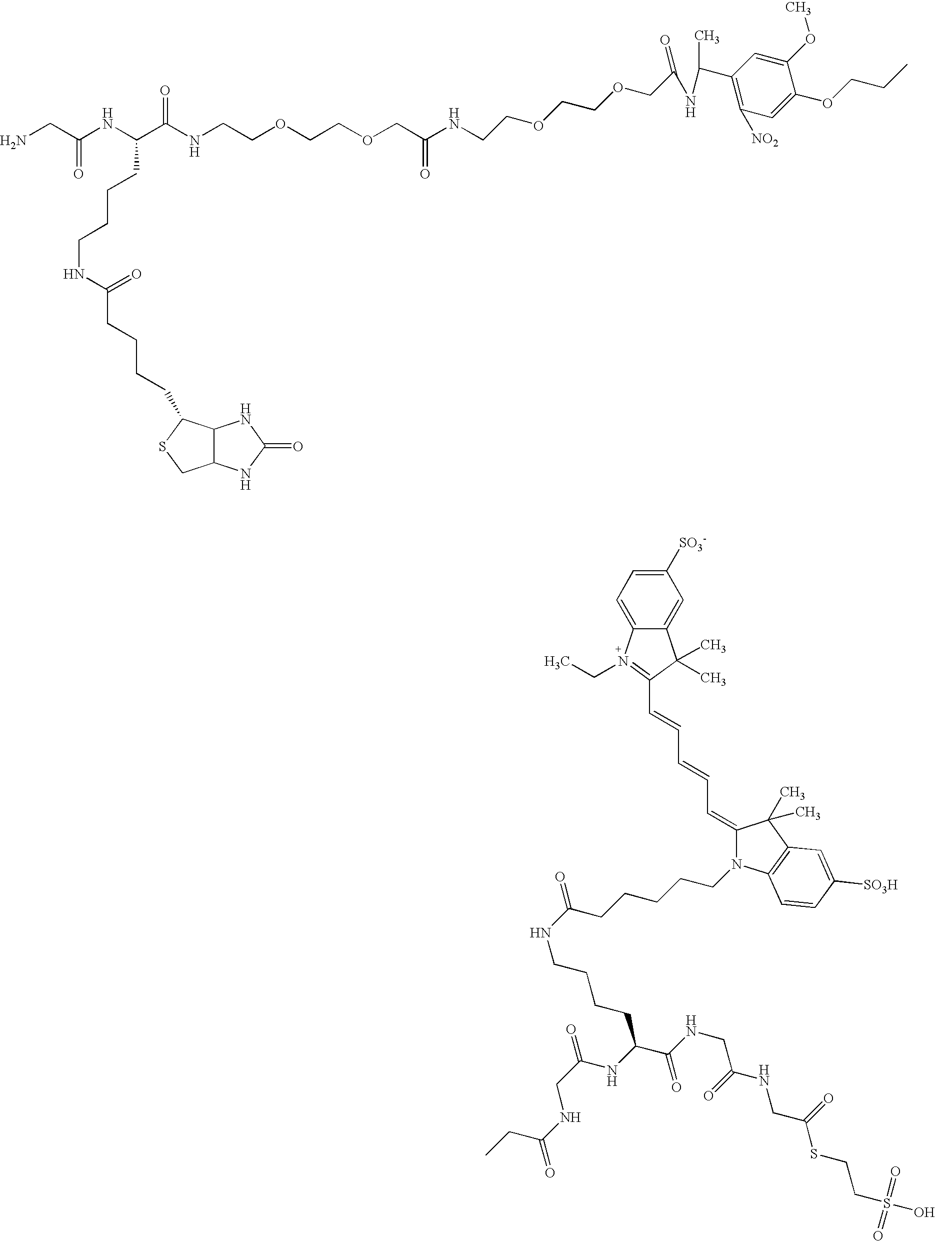

mw: 2063.47. ESI-MS: n.d. MALDI-TOF MS: [MH]⁺ 2063.0. UV/VIS λₘₐₓ pH 7: 649 nm, sh 605 nm.

### EXAMPLE 29

A compound of formula

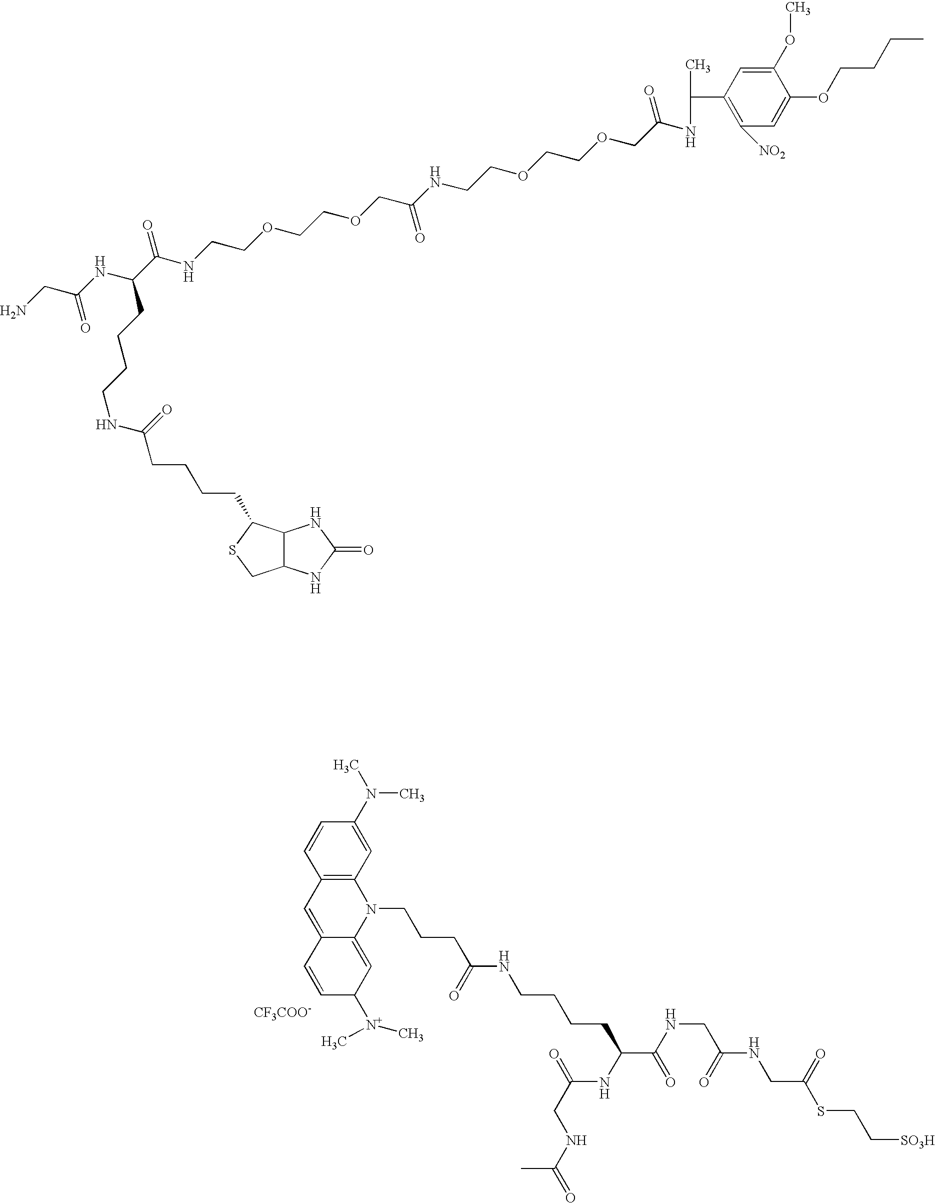

mw: 1758.1. ESI-MS: [M2HK]³⁺ 598.9, [MHKNa]³⁺ 606.4, [MHNa]²⁺ 889.8. MALDI-TOF MS: [MH]⁺ 1757.7. UV/VIS λₘₐₓ pH 7: 503 nm.

### EXAMPLE 30

A compound of formula

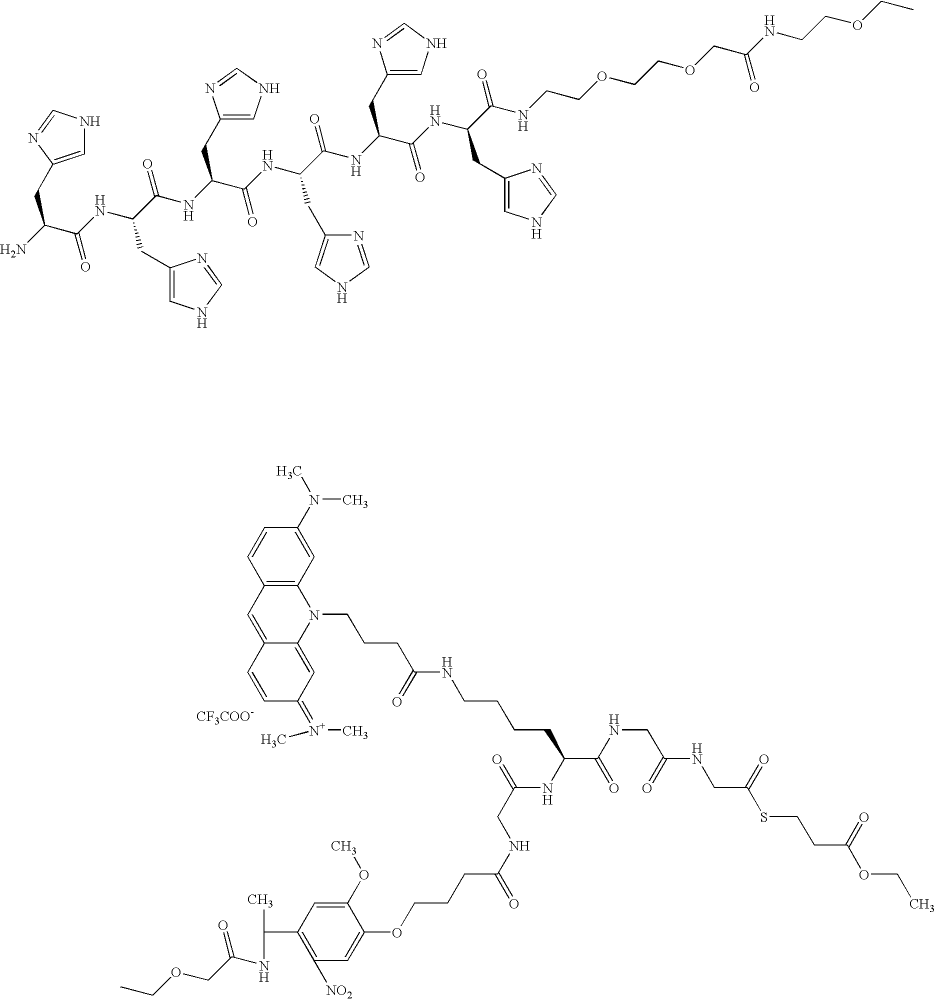

mw: 2161.43. ESI-MS: [M3H]³⁺ 720.5, M4H]⁴⁺ 540.8, [M2H]²⁺ 1080.5. MALDI-TOF MS: [MH]⁺ 2160.8. UV/VIS λₘₐₓ pH 7: 502 nm.

### EXAMPLE 31

A compound of formula

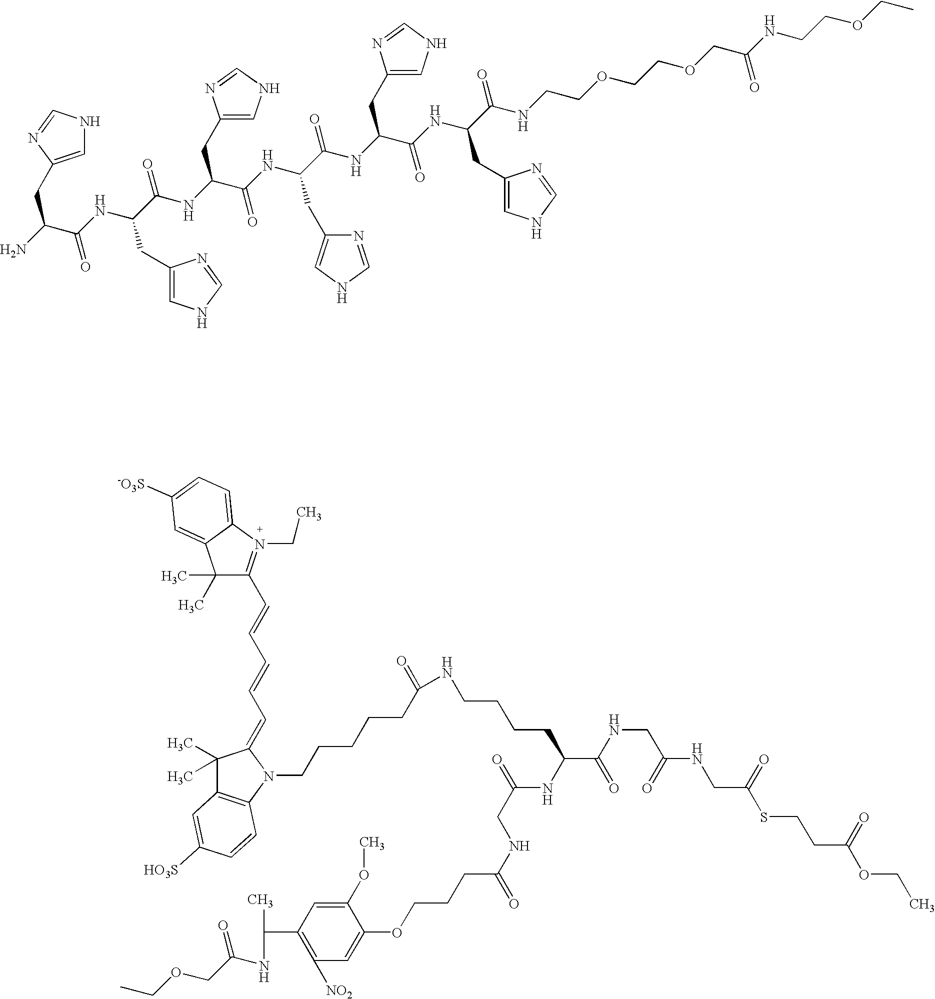

mw: 2466.8. ESI-MS: [M2HCH₃OH]³⁺ 832.1, [M3HCH₃OH]⁴⁺ 624.4. MALDI-TOF MS: [MH]⁺ 2464.0. UV/VIS λₘₐₓ pH 7: 649 nm, sh 605 nm

### EXAMPLE 32

A compound of formula

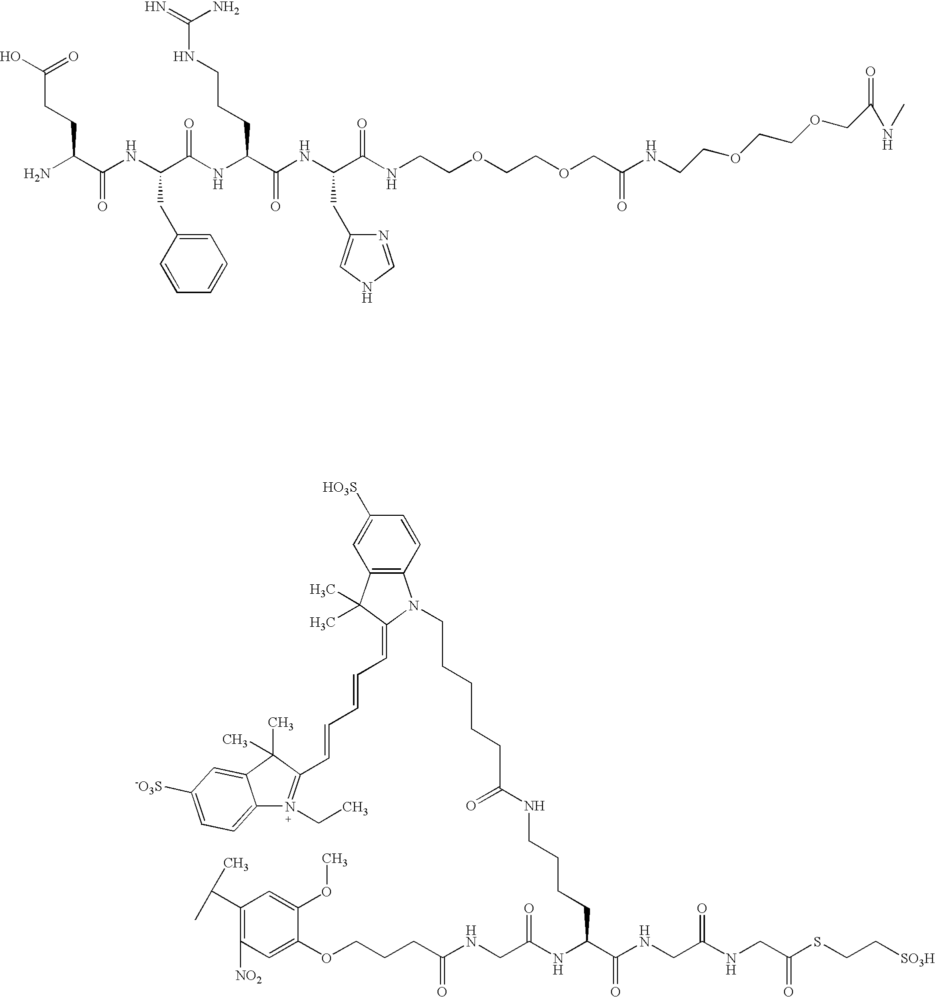

mw: 2221.57. ESI-MS: [M3Na]³⁺ 765.8, [M4Na]⁴⁺ 578.5. MALDI-TOF MS: n.d. UV/VIS λₘₐₓ pH 7: 649 nm, sh 605 nm.

### EXAMPLE 33

A compound of formula

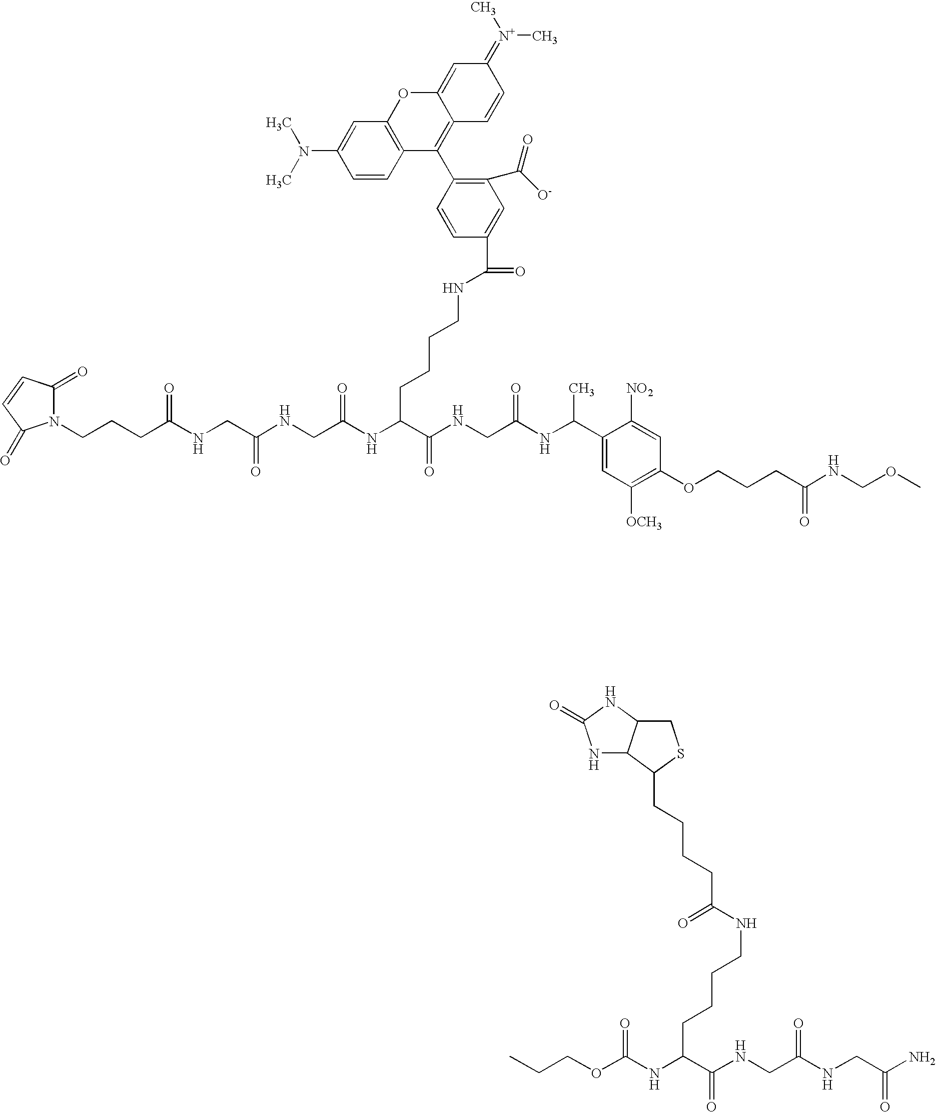

mw: 1788.99. ESI-MS: [M2NaH]³⁺ 611.3 [MNaH]²⁺ 905.3, [M2H]²⁺ 894.3. MALDI-TOF MS: [MH]⁺ 1788.7. UV/VIS λₘₐₓ pH 7: 555 nm, sh 522 nm.

1. A method for providing a labeled target protein or labeled target peptide, which method comprises the steps of:
a. contacting a chemical molecule, which chemical molecule comprises, spacer and/or linker residues, and optionally comprises the following parts
a target protein or a target peptide residue which is covalently bound as one of both terminal groups of said chemical molecule to a chemical backbone, which chemical backbone consists of amino acids and/or spacers and optionally linkers, covalently bound to each other, and which backbone contains a spacer and/or linker residues, and optionally contains
one amino acid or spacer residue as the second of both terminal groups of said chemical molecule covalently bound to a reversible or irreversible affinity tagging residue, with the proviso that in case that the affinity tagging residue is an irreversible affinity tagging residue, there is at least one linker residue covalently bound to said amino acid or spacer residue, which amino acid or spacer residue is on the other hand covalently bound to the irreversible affinity tagging residue, and
one amino acid or spacer residue covalently bound to a labeling residue which labeling residue may be detected by physical means,
with an affinity support, to obtain said chemical molecule bound to the affinity support via the affinity tag of the affinity tagging residue,
b. removing impurities in the reaction mixture surrounding the affinity support to which the chemical molecule according to step a. is bound, and
c. cleaving or eluting said chemical molecule from said affinity support to obtain
(i) in case of an irreversible affinity tagging residue, a labeled target protein or labeled peptide comprising a chemical backbone, which backbone contains a spacer and/or linker residues, and optionally contains one amino acid or spacer residue covalently bound to a labeling residue which labeling residue may be detected by physical means,
(ii) in case of a reversible affinity tagging residue, a chemical backbone covalently bound to a protein or a peptide residue, which backbone contains a spacer and/or linker residues, and optionally contains
one amino acid or spacer residue covalently bound to a labeling residue which labeling residue may be detected by physical means,
one amino acid or spacer residue covalently bound to a labeling residue, which labeling residue may be detected by physical means, and which labeling residue or spacer residue is covalently bound to a reversible affinity tagging residue.

2. A method for providing a labeled target protein or labeled target peptide, which method comprises a method of claim 1, comprising the steps of
a. reacting a compound, which compound comprises, a said spacer and/or linker residues, and optionally comprises the following parts
one amino acid or spacer residue as one of both terminal groups of said chemical molecule covalently bound to a reversible or irreversible affinity tagging residue, with the proviso that in case that the affinity tagging residue is an irreversible affinity tagging residue, there is at least one linker residue covalently bound to said amino acid or spacer residue, which amino acid or spacer residue is on the other hand covalently bound to the irreversible affinity tagging residue,
one amino acid or spacer residue covalently bound to a labeling residue which labeling residue may be detected by physical means,
one amino acid or spacer residue covalently bound to a reactive group containing residue, which residue is covalently bound as one of both terminal groups of said compound, and wherein the reactive group is able to react with a functional group contained in a target protein or target peptide,
with a target peptide or target protein,
b. contacting a chemical molecule obtained in step 1. with an affinity support, to obtain said chemical molecule bound to the affinity support via the affinity tag of the affinity tagging residue,
c. removing impurities in the reaction mixture surrounding the affinity support to which the chemical molecule according to step a. is bound, and
d. cleaving or eluting said chemical molecule from said affinity support to obtain
(i) in case of an irreversible affinity tagging residue, a labeled target protein or labeled target peptide comprising a chemical backbone, which backbone contains, optionally beside spacer and/or linker residues, one amino acid or spacer residue covalently bound to a labeling residue which labeling residue may be detected by physical means,
(ii) in case of a reversible affinity tagging residue, a chemical backbone covalently bound to a protein or a peptide residue, which backbone contains, said spacer and/or linker residues, and optionally contains
one amino acid or spacer residue covalently bound to a labeling residue which labeling residue may be detected by physical means, and
one amino acid or spacer residue covalently bound to a reversible affinity tagging residue.

3. A compound, which compound comprises the following parts
a. a reactive group containing residue, which residue is covalently bound as one of both terminal groups of said compound, and wherein the reactive group is selected from the group consisting of thiol, halogen, imine, aldehyde, the double bond in a vinylsulfonyl or a maleimido group and a group which forms together with a carbonyl group of said residue a carboxylic acid derivative;
b. a label containing residue, which label may be detected by physical means;
c. an affinity tagging group containing residue, which residue is covalently bound as the second of both terminal residues in a CHEMICAL COMPOUND, and wherein said affinity tagging group is a reversible affinity tagging group or an irreversible affinity tagging group, with the proviso that in case that the affinity tagging group is an irreversible affinity tagging group, there is at least one linker residue covalently bound to an amino acid or spacer residue, which carries the irreversible affinity tagging group, and
which residue of any of a., b., or c. is selected from
1 to 10 amino acids, which amino acids are covalently bound to each other via amide bonds of the carboxylic acid group of one amino acid and the amine group of a second amino acid,
1 to 10 spacers, which spacers are covalently bound to each other via functional groups of said spacers, and
1 to 10 amino acids and spacers, which amino acid and spacers are covalently bound to each other either via amide bonds of the carboxylic acid group of one amino acid and the amine group of a second amino acid, or via functional groups of said spacers, or via an amine group or a carboxylic acid group of an amino acid with a functional group of a spacer;
d. optionally further spacer residues; and
e. optionally linker residues.

4. A compound according to claim 3, which is a compound of formula
A-Y-D-Y′—X—Y″-E I
wherein
A is
a group of formula 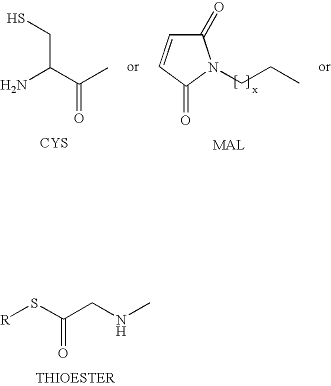
wherein x is 1 to 5, and
R is (C₁₋₄)alkyl carboxylic acid ester, e.g. propionic acid ethyl ester, or a group of formula 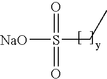
wherein y is 2 to 4, e.g. 2;
D is
a group of formula 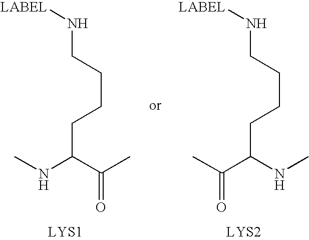
or a group of formula 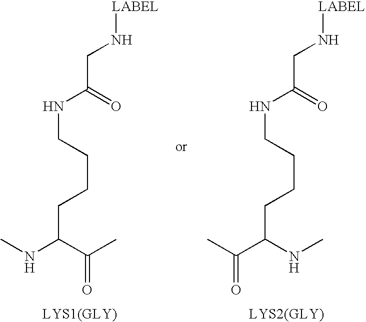
or a group of formula 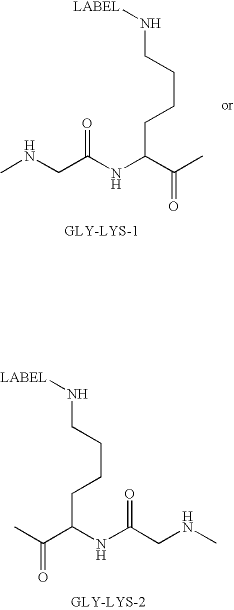
or a group of formula 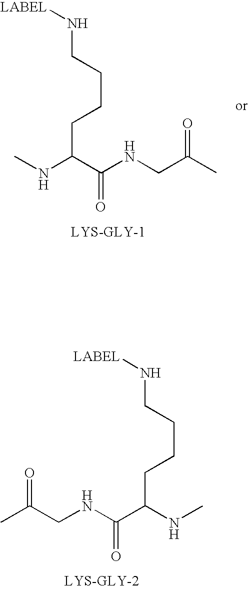
or a group of formula 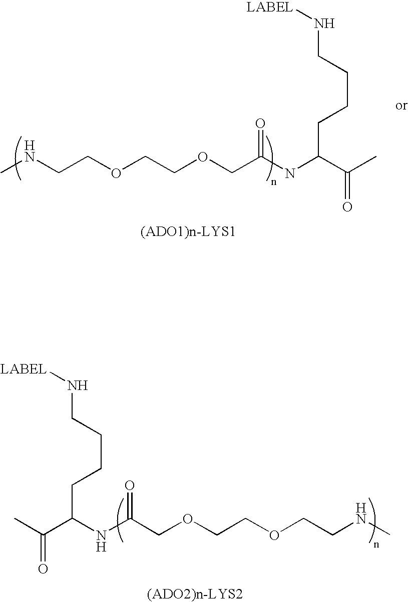
LABEL is a group of formula 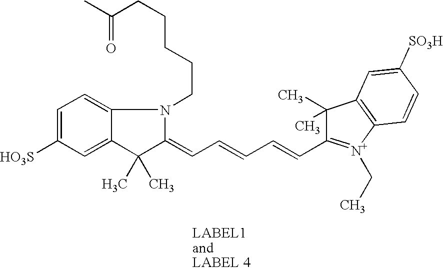
or a group of formula 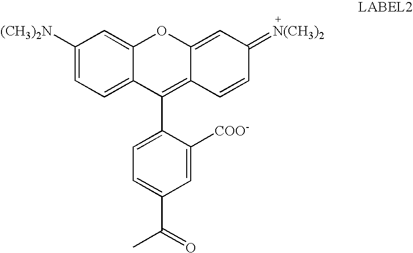
or a group of formula 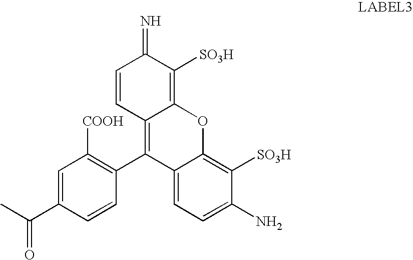
or a group of formula 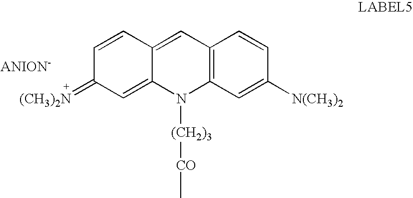
wherein ANION is a negatively charged group, e.g. an anion, such as CF₃COO⁻,
X is a group of formula 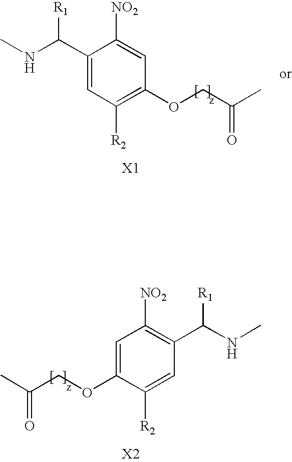
wherein
R₁ is H or CH₃, R₂ is H or —OCH₃ and z is 1 to 5;
E is a group of formula 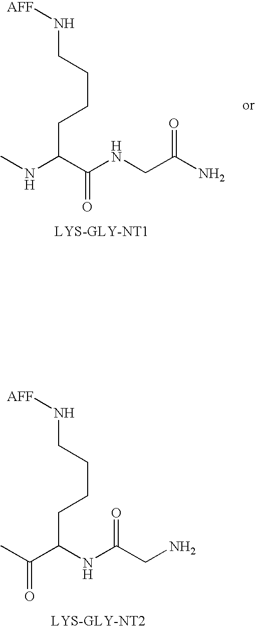
or a group of formula 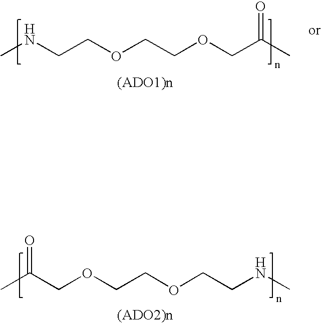
wherein to one of the amine groups a group AFF is bound and wherein n is 1 to 4, and
EITHER the affinity tag is bound to a carboxylic acid residue, e.g. the carboxylic residue of (ADO1)n and is a group of formula 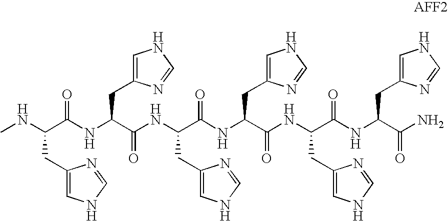
or of formula 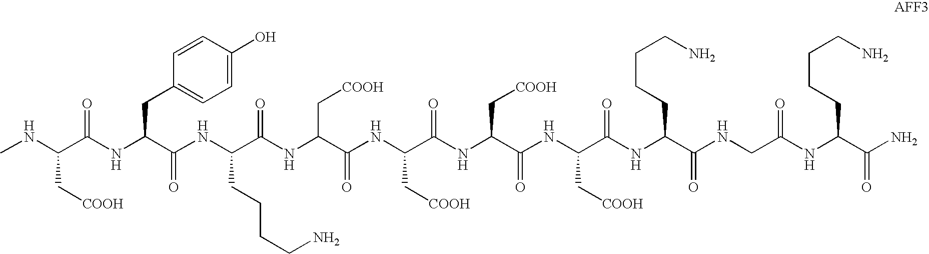
or of formula 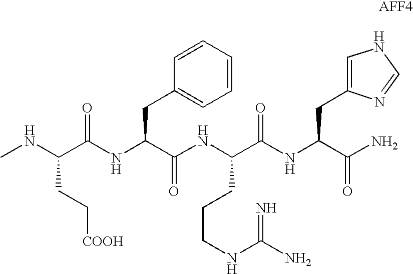
OR the affinity tag is bound to an amino group, e.g. to the ε-amino group of the lysine residue in LYS-GLY-NT1 or LYS-GLY-NT2; or to an amino group of (ADO1)n and is a group of formula 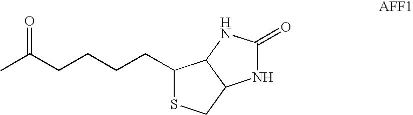
or of formula 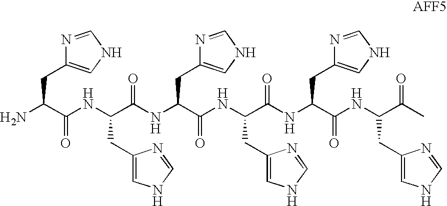
or of formula 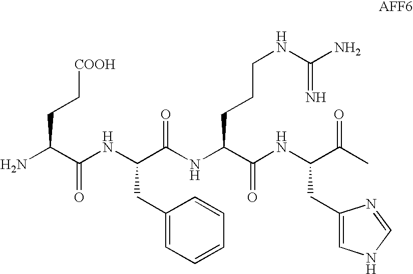
and
Y, Y′, Y″ independently of each other are a group of formula 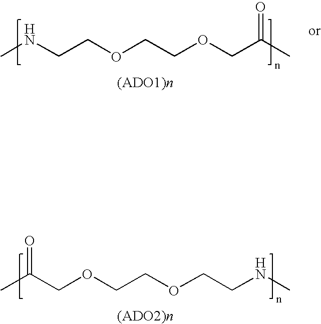
wherein n is 1 to 4,
or a group of formula 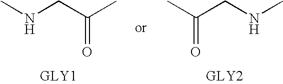
with the proviso, that
IF A is CYS or MAL,
THEN
D is LYS1, LYS1 (GLY), GLY-LYS1, LYS-GLY1 or (ADO1)ₙ-LYS1,
X is X1,
E is LYS-GLY-NT1 or (ADO1)n,
AFF is AFF1, AFF2, AFF3 or AFF4, and
Y, Y′and Y″independently of each other are GLY1 or (ADO1)n, wherein n is 1 to 4, and
IF A is THIOESTER,
THEN
D is LYS2, GLY-LYS1, LYS-GLY2 or (ADO2)ₙ-LYS1,
X is X2,
E is LYS-GLY-NT2 or (ADO2)n and n is 1 to 4,
AFF is AFF1, AFF5 or AFF6, and
Y, Y′ and Y″ independently of each other are GLY2 or (ADO2)n, wherein n is 1 to 4.

5. A compound according to claim 4 wherein A, Y, D, Y′, X, Y″ and E are as defined in TABLE 1 below, wherein in X1 and X2, R₁ is methyl, R₂ is methoxy and z=3, and in (ADO1)n and (ADO2)n n=1, except for EX 12 wherein n=3; and for EX 11, 15, and 26 to 29 wherein n=2:
TABLE 1

| EX | A | Y | D | LABEL | Y′ | X | Y″ | E | AFF |
|---|---|---|---|---|---|---|---|---|---|
| 8 | CYS | GLY1 | LYS1 | LABEL 1 | GLY1 | X1 | ADO1 | LYS- | AFF1 |
| | | | | | | | | GLY- | |
| | | | | | | | | NT1 | |
| 9 | CYS | GLY1 | LYS1 | LABEL2 | GLY1 | X1 | ADO1 | LYS- | AFF1 |
| | | | | | | | | GLY- | |
| | | | | | | | | NT1 | |
| 10 | CYS | GLY1- | ADO1- | LABEL1 | GLY1 | X1 | ADO1 | LYS- | AFF1 |
| | | ADO1 | LYS1 | | | | | GLY- | |
| | | | | | | | | NT1 | |
| 11 | CYS | GLY1 | LYS1 | LABEL1 | GLY1 | X1 | (ADO1)₂ | LYS- | AFF1 |
| | | | | | | | | GLY- | |
| | | | | | | | | NT1 | |
| 12 | CYS | GLY1 | LYS1 | LABEL1 | GLY1 | X1 | (ADO1)₃ | LYS- | AFF1 |
| | | | | | | | | GLY- | |
| | | | | | | | | NT1 | |
| 13 | CYS | GLY1 | LYS1 | LABEL1 | GLY1 | X1 | — | ADO1 | AFF2 |
| 14 | CYS | GLY1 | LYS1 | LABEL1 | GLY1 | X1 | ADO1 | ADO1 | AFF2 |
| 15 | CYS | GLY1 | LYS1 | LABEL1 | GLY1 | X1 | (ADO1)₂ | ADO1 | AFF2 |
| 16 | CYS | GLY1— | LYS1 | LABEL2 | GLY1 | X1 | ADO1 | LYS- | AFF1 |
| | | GLY1 | (GLY) | | | | | GLY- | |
| | | | | | | | | NT1 | |
| 17 | CYS | GLY1 | LYS1 | LABEL1 | GLY1 | — | ADO1 | ADO1 | AFF3 |
| 18 | CYS | GLY1 | LYS1 | LABEL1 | GLY1 | X1 | — | ADO1 | AFF3 |
| 19 | CYS | GLY1 | LYS1 | LABEL1 | GLY1 | X1 | ADO1 | ADO1 | AFF3 |
| 20 | CYS | GLY1 | LYS1 | LABEL3 | GLY1 | X1 | ADO1 | ADO1 | AFF2 |
| 21 | CYS | GLY1 | LYS1 | LABEL4 | GLY1 | X1 | ADO1 | ADO1 | AFF4 |
| 22 | MAL | GLY1— | LYS1 | LABEL4 | GLY1 | X1 | ADO1 | ADO1 | AFF4 |
| | | GLY1 | | | | | | | |
| 23 | CYS | GLY1 | LYS1 | LABEL5 | GLY1 | X1 | ADO1 | ADO1 | AFF2 |
| 24 | CYS | GLY1 | LYS1 | LABEL3 | GLY1 | — | ADO1 | ADO1 | AFF2 |
| 25 | CYS | GLY1 | LYS1 | LABEL1 | GLY1 | — | ADO1 | ADO1 | AFF2 |
| 26 | THIOESTER; | GLY2 | LYS2 | LABEL1 | GLY2 | X2 | (ADO2)₂ | LYS- | AFF1 |
| | R = (CH₂)₂—CO— | | | | | | | GLY- | |
| | OEt | | | | | | | NT2 | |
| 27 | THIOESTER | GLY2 | LYS2 | LABEL5 | GLY2 | X2 | (ADO2)₂ | LYS- | AFF1 |
| | R = (CH₂)₂—CO— | | | | | | | GLY- | |
| | OEt | | | | | | | NT2 | |
| 28 | THIOESTER | GLY2 | LYS2 | LABEL1 | GLY2 | X2 | (ADO2)₂ | LYS- | AFF1 |
| | R = (CH₂)₂—SO₃H | | | | | | | GLY- | |
| | | | | | | | | NT2 | |
| 29 | THIOESTER | GLY2 | LYS2 | LABEL5 | GLY2 | X2 | (ADO2)₂ | LYS- | AFF1 |
| | R = (CH₂)₂—SO₃H | | | | | | | GLY- | |
| | | | | | | | | NT2 | |
| 30 | THIOESTER | GLY2 | LYS2 | LABEL5 | GLY2 | X2 | ADO2 | ADO2 | AFF5 |
| | R = (CH₂)₂—CO— | | | | | | | | |
| | OEt | | | | | | | | |
| 31 | THIOESTER | GLY2 | LYS2 | LABEL1 | GLY2 | X2 | ADO2 | ADO2 | AFF5 |
| | R = (CH₂)₂—CO— | | | | | | | | |
| | OEt | | | | | | | | |
| 32 | THIOESTER | GLY2 | LYS2 | LABEL1 | GLY2 | X2 | ADO2 | ADO2 | AFF6 |
| | R = (CH₂)₂—SO₃H | | | | | | | | |
| 33 | MAL | GLY1 | GLY- | LABEL2 | GLY1 | X1 | ADO1 | LYS- | AFF1 |
| | | | LYS1 | | | | | GLY- | |
| | | | | | | | | NT1 | |

and wherein the groups
CYS, MAL, THIOESTER, GLY1, GLY2, LYS1, LYS1(GLY), LYS2, LABEL1, LABEL2, LABEL3, LABEL4, LABEL5, X1, X2, (ADO1)n, (ADO2)n, LYS-GLY-NT1, LYS-GLY-NT2, AFF1, AFF2, AFF3, AFF4, AFF5 and AFF6 are as defined in claim 4.

6. A compound according to claim 3, which is covalently bound to the residue of a target protein or a target peptide via a bond originating from the reaction of a reactive group with a functional group of said target protein or target peptide.

7. A compound according to claim 6, which compound is further bound to an affinity support via an affinity tagging group.

8. A compound according to claim 3 in the form of a salt.

9. A compound according to claim 3 bound to a solid support via a terminal functional group.

10. The use of a compound of according to claim 3 for the labeling of a target protein or target peptide.

11. The use of a target protein or target peptide bound to a compound according to claim 6, in a high throughput screening assay.

12. A kit comprising a compound of claim 3 and instructions for using the kit.

13. A method for identifying an agent that modulates the activity or characteristic of a target protein or a target peptide comprising the steps
a. contacting a target protein or target peptide bound to a compound according to claim 6, with a candidate compound, which is expected to modulate the activity or characteristics of said target protein or target peptide,
b. measuring a signal,
(i) in the absence of such candidate compound, and
(ii) in the presence of such candidate compound
and determining whether there is a difference in the signals measured in steps (i) and (ii), and
c. choosing an agent determined in step b.

14. A pharmaceutical composition comprising an agent identified in a method of claim 13 in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers, e.g. further comprising another pharmaceutically active agent.

15. An agent identified by a method of claim 13 for use as a pharmaceutical.

16. A method of treatment disorders mediated by a target protein or target peptide in vivo, comprising administering to a subject in need of such treatment an effective amount of an agent identified by a method according to the present invention.

17. A kit comprising a compound of claim 6, and instructions for using the kit.


SEQUENCE LISTING (3 sequences)

SEQ ID NO: 1 - protein, 10 aa, artificial
  His6 affinity tag and linker for affinity purification with Ni2+ column
CGKGHHHHHH

SEQ ID NO: 2 - protein, 32 aa, Homo Sapiens
CSNLSTCVLGKLSQELHKLQTTPRTNTGSGTP

SEQ ID NO: 3 - protein, 110 aa, Homo Sapiens
NFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEK